SEQUENCE LISTING (59 sequences)

SEQ ID NO: 1 - protein, 659 aa, Solanum tuberosum
MAAAASPSPCFSKTLPPSSSKSSTILPRSTFPFHNHPQKASPLHLTHTHHHRRGFAVSNVVISTTTHNDVSEPETFVSRFAPDEPRKGCDVLVEALEREGVTDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIIRLISESKKPVLYVGGGCLQSSEELRRFVELTGIPVASTLMGLGAFPTGDELSLQMLGMHGTVYANYAVDGSDLLLAFGVRFDDRVTGKLEAFASRAKIVHIDIDSAEIGKNKQPHVSICADIKLALQGLNSILEGKEGKLKLDFSAWRQELTEQKVKYPLSFKTFGEAIPPQYAIQVLDELTNGNAIISTGVGQHQMWAAQYYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGDPANEEEIFPNMLKFAEACGVPAARVSHRDDLRAAIQKMLDTPGPYLLDVIVPHQEHVLPMIPSGGAFKDVITEGDGRRSY

SEQ ID NO: 2 - protein, 657 aa, Solanum tuberosum
MAAASPSPCFSKNLPPSSSKSSILLPKSTFTFHNHPKNTSPLHLTHTQHHSRFTVSNVILSTTTHNDVSEPEIFVSRFAPDEPRKGCDVLVEALEREGVKDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYISRLPKLPNEMLLEQIVRLISESKKPVLYVGGGCTQSSEELRRFVELTGIPVASTLMGLGTFPCGDELSLQMLGMHGTVYANYAVDSSDLLLAFGVRFDDRVTGKLEAFASRAKIVHIDIDSAEIGKNKQPHVSICADIKLALQGLNSILEGKEGKLKLDFSAWRQELTEQKVKYPLNYKTFGEAIPPQYAIQVLDELTNGNAIISTGVGQHQMWAAQYYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGNPANEEEIFPNMLKFAEACGVPAARVSHRDDLRAAIQKMLDTPGPYLLDVIVPHQEHVLPMIPSGGAFKDVITEGDGRRSY

SEQ ID NO: 3 - protein, 588 aa, Solanum tuberosum
MAAASPSPCFSKNLPPSSSKSSILLPKSTFTFHNHPKNTSPLHLTHTQHHSRFTVSNVILSTTTHNDVSEPEIFVSRFAPDEPRKGCDVLVEALEREGVKDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYISRLPKLPNEMLLEQIVRLISESKKPVLYVGGGCTQSSEELRRFVELTGIPVASTLMGLGTFPCGDELSLQMLGMHGTVYANYAVDSSDLLLAFGVRFDDRVTGKLEAFASRAKIVHIDIDSAEIGKNKQPHVSICADIKLALQGLNSILEGKEGKLKLDFSAWRQELTEQKVKYPLNYKTFGEAIPPQYAIQVLDELTNGNAIISTGVGQHQMWAAQYYKYKKPRQWLTSGGLGAMGFGLPAAIGAAVGRPGEIVVDIDEACGVPAARVSHRDDLRAAIQKMLDTPGPYLLDVIVPHQEHVLPMIPSGGAFKDVITEGDGRRSY

SEQ ID NO: 4 - protein, 659 aa, Solanum laciniatum
MAAAASPSPCFSKTLPPSSSKSSTILPRSTFSFHNHPQKASPLHLIHAQHNRRGFAVANVVISTTTHNDVSEPETFVSRFAPDEPRKGCDVLVEALEREGVTDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIVRLISESKKPVLYVGGGCSQSSEELRRFVELTGIPVASTLMGLGAFPTGDELSLQMLGMHGTVYANYAVDSSDLLLAFGVRFDDRVTGKLEAFASRAKIVHIDIDSAEIGKNKQPHVSICADIKLALQGLNSILEGKEGKMKLDFSAWRQELTEQKMKYPLNFKTFGDAIPPQYAIQVLDELTNGNAIISTGVGQHQMWAAQYYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGDPSNEEEIFPNMLKFAEACGVPAARVSHRDDLRAAIQKMLDTPGPYLLDVIVPHQEHVLPMIPSGGAFKDVITEGDGRCSY

SEQ ID NO: 5 - protein, 657 aa, Solanum laciniatum
MAAASPSPCFSKTLPPSSSKSSTLLPKSTFTFHNHPKKASPLHLTHTQHHSRFTVSNVILSTTTHDDVSEPEIFVSRFAPDEPRKGCDVLVEALEREGVKDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIVRLISESKKPVLYVGGGCSQSSEELRRFVELTGIPVASTLMGLGAFPSGDELSLQMLGMHGTVYANYAVDSSDLLLAFGVRFDDRVTGKLEAFASRAKIVHIDIDSAEIGKNKQPHVSICADIKLALQGLNSIFESKKGKLKLDFSAWRQELTEQKVKYPLNFKTFGEAIPPQYAIQVLDELTNGNAIISTGVGQHQMWAAQHYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGNPANEEEIFPNMLKFAEACGVPAARVSHRDDLRAAIQKMLDTPGPYLLDVIVPHQEHVLPMIPSGGAFKDVITEGDGRRSY

SEQ ID NO: 6 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 7 - DNA, 2351 bp, Musa acuminata
cagattaaaaaacccctttttttcgtcccccaaatggcttcctctgcggcggcggcggtggctgctgcgggagccgtgatcaccccatcgaagtcatcccacccgcccttctccgctgcctaccgcctccccttccccctccccaagcccctccattcactctccacccgccacccccacgtccggcccatctccgcctccgcggaccgccgccagctcccctccgctgccgcccccactgccgatgccacgaccgcccctctcctccgtaacttcgccccggatgagccccgcaaaggcgccgacatcctagtcgaggcccttgagcgggagggcgtcaccgaccttttcgcctaccccggcggcgcctccatggagatccatcaggctctcacccgctcgccatctatcaccaaccacctcctccggcacgagcagggggaggtcttcgctgcctctggatacgcccgctccactggtcgccctggcgtgtgcatcgccacctccggccctggcgccaccaatctcgtctccggtctcgccgatgccctccttgactccgtccctctcgtcgccatcaccggccaggtccctcgccgcatgatcggcaccgacgctttccaggagacccccatcgttgaggtcaccagatccatcaccaagcacaactacctggtcctcaacgttgatgacattccccgcatcattaaagaagccttcttcgtagccaccacgggccgccctggcccggtgctcgttgacatccccaaggacatccagcagcagcttgccatccctgtatgggacccaccgctccgccttcccggctacatctcccgcctccccaggcttcctgcacgccctctgctcgatcagatcctccgcctggtgtcggaatcccgtcgcccagtcctctatgttggtggtggctgcttgaactctagcgaggagcttcatcggtttgcagaccttactggcatccccattgcaagtaccctaatgggtctcggggcctatcccaccgatgctgagctatcattgaagatgttgggaatgcatgggacggtctatgccaactatgccatcgataaagctgatctgctgcttgcatttggtgtaaggtttgatgaccgtgtgactggaaagattgaagcttttgctagcagagcaaagattgtgcacattgacattgatccggctgagatcgggaagaacaagcagccacacgtttcaatctgtgctgatgtgaagttggcattgcagggaatgaatgcattgatggaggagactgagatttatcgaaagtttgacttttcgacctggagagaagaactggacaagcagaagaagatatacccattaaactataagactttcggggatcagattcctccccagtatgcaattcaggtgcttgatgagctaacgaatggggaggcaatcattactactggtgttgggcagcaccagatgtgggccgcacaatattactcatacaagagggcacggcagtggctgacatctgccggactaggtgctatgggtttcggtttaccagcagcagctggtgcagctgttgggaatccaggcgttactgtcgtggacattgatggggatgggagtttccagatgaatgctcaggaactggcaatgattcggattgagaatcttcctgtcaagattatggtgttgaacaaccagcatttgggcatggtcgtgcaatgggaagatcgattctaccatagaaacagggcacatacttatctggggaacccagcaaatgagactgaggtattccctgatttcttgaaaatctcagaggggtatggcatacctgctgctcgtgtcacgaagaagagtgaggttagagaagcaatcaggaagatgctggaaacatcggggccttacttgttggatgtgatcgtgccacatgaggaacatgtgttgcctatgattccaagtggcggtgcgtttaaagatatgatacttgatggagatggaagaacaccatattagtgctttttaagataggtaatcagttcagttctgttgtggcattgagtatcagttctgttctgtcgtggcttgaactatgtatttaggacactatagtactatctttcctgttggtatgatgagtttttctctggctttggtcatatctatgaactacgaaatgttatagtcatgtctacgacctacgaggactggtcaatctttttcttccgtcctgttgtgtgttttgatcattagttattacatagtttgttcgctatcacttatttggtattgctgggtgtccttgttgcattttcagggattttgttttataagcttaagaataatttcct

SEQ ID NO: 8 - DNA, 2177 bp, Musa acuminata
cctctcgctcgctcgctcgctcgctcctcagggaacccagtatggcctcctctacgccgggggccgccatcgctccctcgaatccctcgctctggccattctccgctgcatcccgtctcttggtccccttccccaagcctctcctgtccctctccccgagccaccgcaatgttcggcccatctccgcgtccgcggaccgccaccagaccccctccgccgccgcctccaccgccgctaccaccgctgtccccctcctccgtaactttgcccccgacgaaccccgcaagggctctgacatccttgtcgaggccctcgagcgggagggcgtcaccgatctgttcgcctaccctggaggcgcctccatggagatccaccaggccctcacccgctccccatccatcaccaatcacctcctccgccacgagcagggcgagatcttcgccgcctccggctacgcccgctccaccggccgcccgggcgtgtgcatagccacctccggccccggcgctaccaacctcgtctccggcttcgcggacgcgctcctcgactccgtccccctcgtcgccatcaccggccaggttccccgccgcatgatcggcacggacgccttccaggagacccccattgtcgaggtcaccagatccatcaccaaacacaactacctggtccttaacgtcgatgacattccccgcatcattaaagaagccttctttctcgccagcactggccgccctggcccggtgctggtcgacatccccaaggacattcagcagcagcttgccatccctgtttgggacccgccgctacgccttcccggatacatctcccgcctccctaagcctccttcacgctgtctgcttgaccaaatcatccgccttgtgtccgaatcccatcgcccggttctctatgttggtggcggctgcttgaactccagcgaggagcttcgccggtttgcggaccttactggcatccccatcgcgagtaccctgatgggtctcggggtctatcccaccgatgcggagctatcgttgaagatgttgggaatgcacgggactgtctatgccaactattcggtcgataaagctgacctcttgctcgcacttggcgtcaggttcgacgatcgtgtgactggaaagcttgaagcttttgctagcagggcaaagattgtgcacatcgatatagatccggctgagatcgggaagaacaagcagccacatgtttcattatgtgctgatgtgaggttggctctgcaggggatgaatgcattgatggaggagagtgggattcatcaaaagttcgatttttccacctggaggaaagagctggaccaactgaagaaggcatacccattgagctataagacttttggggatctgattcctccccagtatgcaattcaggtgcttgacgaactgacgaatggggaagcaatcatttccactggtgttgggcagcaccagatgtgggccgcacagtattactcatacaagagggcacgccagtggctgacatcgggcggactaggtgctatggggtttggtttaccagcagctgccggtgcagccgttgggaacccaggagttactgtcgtcgacattgatggggatgggagtttccagatgaatgctcaggagttggctatgattcggatagagaatctccctgtcaagatgatggtgttgaacaaccagcatttgggcatggttgtacaatgggaggatcgattctaccgtagcaacaggggacatacttacttggggaacccagcaaacgagagtgagatatttcctgatttcttgaaaatcacagaagcgtatggtatacctgctgcccgtgtgacaaagaagagcgaggtcagggaagcaatcaggaagatgctgaagacaccaggaccttacttgttggatgtgatcgtgccacatgaggagcatgtgttgcctatgattcctagtggcggtgcatttaaggatatgatcctcgatggagatggaagaacgccaccacactaatacattttaatttaggtaatcagttctgttctgttatggcttcgaactgtgaagtcaggaaatatgtagtgctggctttcgtgttgttacaatgagctttggctatagtcacatctgtgaactttgagaattggttgacctatttttcctgtctttttgtgcattttg

SEQ ID NO: 9 - protein, 660 aa, Musa acuminata
MASSAAAAVAAAGAVITPSKSSHPPFSAAYRLPFPLPKPLHSLSTRHPHVRPISASADRRQLPSAAAPTADATTAPLLRNFAPDEPRKGADILVEALEREGVTDLFAYPGGASMEIHQALTRSPSITNHLLRHEQGEVFAASGYARSTGRPGVCIATSGPGATNLVSGLADALLDSVPLVAITGQVPRRMIGTDAFQETPIVEVTRSITKHNYLVLNVDDIPRIIKEAFFVATTGRPGPVLVDIPKDIQQQLAIPVWDPPLRLPGYISRLPRLPARPLLDQILRLVSESRRPVLYVGGGCLNSSEELHRFADLTGIPIASTLMGLGAYPTDAELSLKMLGMHGTVYANYAIDKADLLLAFGVRFDDRVTGKIEAFASRAKIVHIDIDPAEIGKNKQPHVSICADVKLALQGMNALMEETEIYRKFDFSTWREELDKQKKIYPLNYKTFGDQIPPQYAIQVLDELTNGEAIITTGVGQHQMWAAQYYSYKRARQWLTSAGLGAMGFGLPAAAGAAVGNPGVTVVDIDGDGSFQMNAQELAMIRIENLPVKIMVLNNQHLGMVVQWEDRFYHRNRAHTYLGNPANETEVFPDFLKISEGYGIPAARVTKKSEVREAIRKMLETSGPYLLDVIVPHEEHVLPMIPSGGAFKDMILDGDGRTPY

SEQ ID NO: 10 - protein, 655 aa, Musa acuminata
MASSTPGAAIAPSNPSLWPFSAASRLLVPFPKPLLSLSPSHRNVRPISASADRHQTPSAAASTAATTAVPLLRNFAPDEPRKGSDILVEALEREGVTDLFAYPGGASMEIHQALTRSPSITNHLLRHEQGEIFAASGYARSTGRPGVCIATSGPGATNLVSGFADALLDSVPLVAITGQVPRRMIGTDAFQETPIVEVTRSITKHNYLVLNVDDIPRIIKEAFFLASTGRPGPVLVDIPKDIQQQLAIPVWDPPLRLPGYISRLPKPPSRCLLDQIIRLVSESHRPVLYVGGGCLNSSEELRRFADLTGIPIASTLMGLGVYPTDAELSLKMLGMHGTVYANYSVDKADLLLALGVRFDDRVTGKLEAFASRAKIVHIDIDPAEIGKNKQPHVSLCADVRLALQGMNALMEESGIHQKFDFSTWRKELDQLKKAYPLSYKTFGDLIPPQYAIQVLDELTNGEAIISTGVGQHQMWAAQYYSYKRARQWLTSGGLGAMGFGLPAAAGAAVGNPGVTVVDIDGDGSFQMNAQELAMIRIENLPVKMMVLNNQHLGMVVQWEDRFYRSNRGHTYLGNPANESEIFPDFLKITEAYGIPAARVTKKSEVREAIRKMLKTPGPYLLDVIVPHEEHVLPMIPSGGAFKDMILDGDGRTPPH

SEQ ID NO: 11 - DNA, 20 bp, Artificial Sequence
  synthetic oligonucleotide
ggtcttcgctgcctctggat

SEQ ID NO: 12 - DNA, 21 bp, Artificial Sequence
  synthetic oligonucleotide
ccaccaccaacatagaggact

SEQ ID NO: 13 - DNA, 20 bp, Artificial Sequence
  synthetic oligonucleotide
atctgttcgcctaccctgga

SEQ ID NO: 14 - DNA, 21 bp, Artificial Sequence
  synthetic oligonucleotide
gatggatctggtgacctcgac

SEQ ID NO: 15 - DNA, 22 bp, Artificial Sequence
  synthetic oligonucleotide
tagggattccgacgatttgttt

SEQ ID NO: 16 - DNA, 21 bp, Artificial Sequence
  synthetic oligonucleotide
tagcgtcatcattggctggga

SEQ ID NO: 17 - DNA, 5214 bp, Artificial Sequence
  synthetic polynucleotide
atgccaaagaagaagaggaaggtttcatcggagacgggccctgttgctgttgaccccaccctgcggcggagaatcgagccacacgagttcgaggtgttcttcgacccaagggagctccgcaaggagacgtgcctcctgtacgagatcaactggggcggcaggcactccatctggaggcacaccagccaaaacaccaacaagcacgtggaggtcaacttcatcgagaagttcaccaccgagaggtacttctgcccaaacacccgctgctccatcacctggttcctgtcctggagcccatgcggcgagtgctccagggccatcaccgagttcctcagccgctacccacacgtcaccctgttcatctacatcgccaggctctaccaccacgccgacccaaggaacaggcagggcctccgcgacctgatctccagcggcgtgaccatccaaatcatgaccgagcaggagtccggctactgctggaggaacttcgtcaactactccccaagcaacgaggcccactggccaaggtacccacacctctgggtgcgcctctacgtgctcgagctgtactgcatcatcctcggcctgccaccatgcctcaacatcctgaggcgcaagcaaccacagctgaccttcttcaccatcgccctccaaagctgccactaccagaggctcccaccacacatcctgtgggctaccggcctcaagtccggcagcgagacgccaggcacctccgagagcgctacgcctgaacttaaggacaagaagtactcgatcggcctcgccatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagacggctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagacgatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctcaagacgtacgcccatctcttcgatgacaaggtcatgaagcaactcaagcgccggagatacaccggctgggggaggctgtcccgcaagctcatcaacggcatccgggacaagcagtccggcaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagacgagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagcgcccgctcatcgagacgaacggcgagacgggggagatcgtgtgggacaagggcagggatttcgcgaccgtccgcaaggttctctccatgccccaggtgaacatcgtcaaaaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagcgccccgcagcaaccaagaaggcagggcaagcaaagaagaaaaagacgcgtgactccggcggcagcaccaacctgtccgacatcatcgagaaggagacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctgtcgggggggagcccaaagaagaagcggaaggtgtag

SEQ ID NO: 18 - protein, 1737 aa, Artificial Sequence
  synthetic polypeptide
MPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPELKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKTRDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 19 - DNA, 20 bp, Artificial Sequence
  synthetic oligonucleotide
caggtccctcgccgcatgat

SEQ ID NO: 20 - DNA, 20 bp, Artificial Sequence
  synthetic oligonucleotide
caggttccccgccgcatgat

SEQ ID NO: 21 - DNA, 23 bp, Artificial Sequence
  synthetic oligonucleotide
agagccagctgttgtggacttgg

SEQ ID NO: 22 - DNA, 23 bp, Artificial Sequence
  synthetic oligonucleotide
gttccagaaagggagcgagaagg

SEQ ID NO: 23 - DNA, 23 bp, Artificial Sequence
  synthetic oligonucleotide
tgaacagcctaaccctggcgcgg

SEQ ID NO: 24 - DNA, 23 bp, Artificial Sequence
  synthetic oligonucleotide
gaagcaaaagttcaaacagttgg

SEQ ID NO: 25 - DNA, 23 bp, Artificial Sequence
  synthetic oligonucleotide
ctgcgagaacctcggcctggagg

SEQ ID NO: 26 - DNA, 23 bp, Artificial Sequence
  synthetic oligonucleotide
ggtgccaaagttcggacctttgg

SEQ ID NO: 27 - DNA, 20 bp, Artificial Sequence
  synthetic oligonucleotide
acccaaaggtcgacgacaaa

SEQ ID NO: 28 - DNA, 21 bp, Artificial Sequence
  synthetic oligonucleotide
catgaacgccctcatgttgtt

SEQ ID NO: 29 - DNA, 21 bp, Artificial Sequence
  synthetic oligonucleotide
ttgatgcccacgagcatttga

SEQ ID NO: 30 - DNA, 19 bp, Artificial Sequence
  synthetic oligonucleotide
atccactggccatccttga

SEQ ID NO: 31 - DNA, 20 bp, Artificial Sequence
  synthetic oligonucleotide
aggtgcccatgttctcgaag

SEQ ID NO: 32 - DNA, 20 bp, Artificial Sequence
  synthetic oligonucleotide
aagtaggtcacgtccctccc

SEQ ID NO: 33 - DNA, 19 bp, Artificial Sequence
  synthetic oligonucleotide
tccatcaggctctcacccg

SEQ ID NO: 34 - DNA, 20 bp, Artificial Sequence
  synthetic oligonucleotide
caacatagagaaccgggcga

SEQ ID NO: 35 - DNA, 20 bp, Artificial Sequence
  synthetic oligonucleotide
accgaagcccctcttaaccc

SEQ ID NO: 36 - DNA, 20 bp, Artificial Sequence
  synthetic oligonucleotide
gtatggctgacaccatcacc

SEQ ID NO: 37 - DNA, 20 bp, Artificial Sequence
  synthetic oligonucleotide
cagacaatcggctgctctga

SEQ ID NO: 38 - DNA, 20 bp, Artificial Sequence
  synthetic oligonucleotide
gatgtttcgcttggtggtcg

SEQ ID NO: 39 - DNA, 20 bp, Artificial Sequence
  synthetic oligonucleotide
cctcgttgcttggggagtag

SEQ ID NO: 40 - DNA, 20 bp, Artificial Sequence
  synthetic oligonucleotide
gccatcaccgagttcctcag

SEQ ID NO: 41 - DNA, 20 bp, Artificial Sequence
  synthetic oligonucleotide
atcgacctctcacaactcgg

SEQ ID NO: 42 - DNA, 20 bp, Artificial Sequence
  synthetic oligonucleotide
ttgttgccgatgacctcctc

SEQ ID NO: 43 - DNA, 1884 bp, Musa acuminata
atggggattcccggtgacgagatcctctccagggtcgctacgggcgatggccacggtgagaacacctcgtacttcgatggctggaaggcctacgataatgatcctttccacccgattcataatcccaatggtgtcatccaaatgggactcgcagaaaaccaggtaatgcttgtttctggctctgtccattactttctcctcctcctgctgctgctgctgctaatgggtttcggtctgcctttcctcagctctgcttggacttgatgcgagattggatcaggaagaatccacaggcttctatatgcaccaaggagggcgtttcagagttcgaagccatcgctaacttccaggactaccatggcctgccggacttccgtaaggtaatcaccgtctgcagccataatgcagctcctcgatcccttactcatgcgtgccatgaacgatgagggcacagttggatcgatatgcgttgctatagccgaaaggtaatgacgcgatcatctatggaaatgcacaggccattgccaagttcatggagaaagcgagaggaggacgagccaggttcgacccggagcgcatagtgatgagcggtggagccaccggagctcaagaaacgatcgcattttgtctggccaatcccggggacgccttcctcattccgacgccatactacccagcgtacgtatgcctgttgagtcaacattctgatctctcaagtaattgcgtcgtcaacttccccgttcgaacaaatgttccagccgaccaatcagtcgtgcaatgacccaaacgacagtcaaacttttatctgcctgagcattgaccaaaaccacaccattcaacgtaattgtggtcatgcaatccgacactaaagaacgacatttggttcttctcaggttcgatcgagacttcaggtggagaactggagttcagctcctccctattcgctgccacagtcacgacaacttcaagatcaccgaagccgagcttgctgctgcctaccggaaggcgcgcgactctaagatcagggttaaaggaatactaataaccaacccgtcgaatcctctgggcacaaccatggacagggagacgctaagaaccctagtaagattcgcgaacgaggaaaggatccacctagtctgcgacgagatcttctccggcacagtcttcgacgggccggaatatgtcagtgtggcggagatattgcaagaggatccgtcgacctgcgacggagacctaatccacatcgtctacagcctgtcgaaggacctcggcgtccccggattccgtgtcggcatcatatactcgttcaacgacgcggtggtcagctgcgctcggaggatgtccagcttcggactggtctcgacgcagactcaacgcctgcttgcttccatgctgggagacgacgacttcaccaccgacctcttggcggagagcaggaggagattaatgcacaggcacaggacgtttactgccggcctcgaaggcgtcggcattcgttgcttacagagcaacgccggactattctgctggatgagcttgaagcctctgctgaaagacgccacggcggagggcgaggtcgagctgtggcgggtgatagtgaacgaggtgaagctcaacatctctccggggtcctcgttccactgcaccgagccggggtggttcagggcgtgctttgccaacatggacgaggagaccatggagacggccctgcggcggatcaggacgttcgtgcgccgggcgaacgacgcagctactgccgccaagaccaagaagaggtgggacacatcgcttcgcctgagcttgccacgaaggttcgaggagatgaccgtcctgacaccgcgtctgatgtctcctcgctctccgctcgttcaggccgccacctga

SEQ ID NO: 44 - DNA, 2504 bp, Musa acuminata
gcagcagctgcttctccttcttctctgctcgcttcagccttttccggtacgtacctgagataacgggtcacatgaggatctacggcgaggagcacccaaatcagcagatcctctctcggatcgcgaccaacgacggccatggcgagaactcctcctacttcgatgggtggaaggcctacgagaaggatcctttccacctcaccgacaaccccacgggggtcatccaaatgggactcgcagaaaaccaggttagagttccttcatggtgatgattaatcgcacatgccttccgtcaattgccactccctgcggttgctaatctaatctgtatgtgggttttgggtctttctttcctcagctttccctcgacttgatccgagactggatgaagaagaacccgcaggcttcgatctgcaccgaagaaggggtctcagagttcaaagcaattgccaactttcaggactatcatggcctcccagccttccgaaaggtaatgatttcaacccaaaacgcagcgctgcagctgcttgtcctcactgtccaagtagctacatacgtccaatatgataaagctgggactgacagccacttacggcccgagccctgcctgctcaccctggataagggataagctaatgatggtgtgatttgctgacacgcgcaggccatcgcccagttcatggagaaggtgagagggggacgagccagatttgacccagaccgcatcgtgatgagcggtggagccaccggtgctcaggaaaccatcgccttttgcctggctgatcctggcgaggccttcttgattccaacgccatattatccggggtaagtgttcaggtgtactaatctaccgagttctttatccggcagaggatctaatggcatctgcatggtttccagattcgatcgagacttcaggtggaggacaggagttcagctcctccccattcactgccacagttccaacaagttcaagatcacccaagccgcactggagactgcttacaggaaggctcgaaactcacacattagagtcaaaggaatactggtgaccaacccatcgaaccctctgggcacaaccatggacagagagacgctgagaaccctagtcagcttcgtcaacgagaaaaggatgcacttggtgtgcgacgagatcttctccggaaccgtcttcgacaagccgagttacgtgagcgtctccgaggtgatcgaagacgatccctactgcgacagggatctgattcacatcgcctacagcctctccaaggacctgggcgtccctggcttccgcgtcggcgtcatatactcctacaacgacgccgtggtcagctgcgcgaggaagatgtcgagctttggactggtctcgtcgcagacgcagcacctgctcgcttccatgttgggagacgaggagttcaccacgagtttcttagcgacgagccggacgaggttgtgcgggcggcgcagggtctttacggacggcctcaagcgagtcgggattcattgcttggacggcaacgcggggctgttctgctggatggacttgaggccgttgctgaaggaagcgacggtggaggcggagctccggctgtggcgggtgatcatcaacgacgtgaagctcaacatctcgccggggtcgtccttccactgctcggagccggggtggttcagggtatgcttcgccaacatggacgacacggccatgaagatagcgctgaggaggatcgagagtttcgtgtaccgggagaacgacgccgctgtgcaggcgaagaacaagaggaggtgggacgaagcgctgcggctgagcttgcctcgtcggaggttcgaggatccgaccatcatgacaccacatctgatgtctccccactcgcctctcgttcaagccgccacctgaaacatcgacagcggcgtgtctgatgtcaacgaaggttaattaccgtctgatatgttgcacatttctttgttctttggattatttatttttttttttttgggaaaaatgggttgaatgttcccactaagttatattagattgttgttcggtctcattcatgttataggaaacgaggatagaattgcttgcctctctctttcttttatatatggaaatatgttacaattggcctaagcttatttgatgacattaatttcacaagacaaagccttctaattaatgtttcggaccaaatgcaggagctcactacatacatttgttacacttcatatgttcaaaattagtccagtttaccggtgactcagttttaaaggttataaatggttctgattcaagtacttatctttggttctgttaattggttcaaaccgaatcgattttaatttaaacaatattaatttaattaaattttttaattggtttaaatcgattaatcaaatcagttgatcagggaaaatattattgatgtcttactcaactcgatatggtctatactcacgtgcgtaggaatgtccgagatgtctctgagataaaaacatcgtgctttcgtgat

SEQ ID NO: 45 - DNA, 2294 bp, Musa acuminata
tagctcgtgttctcccttctccccaggcttcccagtactcgcctaagatcgtaacgtcggcaatggggctccacgttgatgaacactcaaattacaatgtcctctccagcatcgcaacgagcgatggccacggggagaactcctcatacttcgatggctggaaggcctacgataatgatcctttccaccccatcgacaatcctcagggggtcatccaaatgggacttgcagaaaaccaggtaaatgctgtttcacaactagttcggtaattatggtagttttttcatggcctatggccaaaaatatgccttccgtattctcctactacttggaatgctaacgggtgctgcgttttccttatctcagctctgcctggacttgatgcagcagtggatcaagcagaacccacaggcttccatttgcaccggcgagggcgtttccgagtttaaggacgtcgcgaacttccaagactaccacggcctgccagacttccgaaaggtaataaccatcacagtgcagctctttagttagtccttatcatgtcataaactgtggaccctcgagaatagattacatcactcagataaaagatgtgcgcattatgactcacgtacatgagtccagaacttgtatctacttgtaacgacgtcaagaggattctggaaatggtgcctgctgggctagggacaacctcactagattgctttgctgtttctgaaaggctaatgatgtgatttgtggaaacacgcaggcgattgctaggttcatggggaaagcgagaggaggaggagctacgttcgacccggagcgcattgtaatgagcggcggagccaccggagctcaggaaaccatcgcattttgtctagcgaatcctggggaggccttcctgattccaacgccatattatccagggtacgtagacctatcctacatcaagattttatgttttatgtatatttcacagtgacactaatctgttttaaagaaaactgtttgaggatgagccgatcgaactacggaggcaacattaatataatccagcttactggtataaccaaaaaattagtagtcaatatttgccatcgcacgactgtgacgtcgacaagacagtctcagtatattatatttcttaattaataacgctacaccaaaaccataaccgacctaccggccgcttgaggtttctgcactctccggcctcattatggatctatcggttgatatatatatatatatatatgacagcgatttcacatttcctgcagcttcgatcgagactttcggtggagaactggagttcaactcctccctattcagtgccacagcttcgacaacttcaagatcaccgaacccgcgctagttactgcctatcaaagggcacaaacagctaacatcagggttaaaggaatcctggtaaccaacccttcaaaccctctgggtacaaccttggacagagagacactgagaaccttagtgagcttcgccaacgagaaacggatccacttggtgtgcgacgagatattctcgggcaccgtcttcgacaagcctacctacgtcagcgtctccgagatcgtggaagaggaaccatactacgacagggacctaattcacatcgtctacagtctgtccaaggatctcggcgtccctggattccgcgtgggtgtcatttactcgtacaatgatgcagtggtcagctgtgctcggaagatgtccagctttggactggtctcgactcaaacgcagcacctactggcttccatgctgggagatgatgacttcacaaccaaatttttggcggagagcaggaggagattgtcgcgcaggcacaaatattttactgctggcctccacagagttgatatcaaatgtttggagagcaatgcggggctattctgctggatgaacttgacgcatctgctaaacgaagccacggtggaggcggaactcaagctgtggcgagtgataattaaggaggtgaagctcaacatttcaccggggtcttcgttccactgctctgagccggggtggttcagggtgtgcttcgccaacatggacgataacaccatggaaaccgcattgaagaggatcaggaagtttgtgtcccccgggaatcacactgcggctgcgcaagccaagaagaagaacaagaggtgggacgcggcgctccgcctaagtttgcctcgtcggttcgaggaactgagcatcatgacacctcgcctcatgtctcctcactcgccccttgttcaggccgccaactgatggtgatggatgagcgtgggcgatattaaccgacg

SEQ ID NO: 46 - DNA, 1384 bp, Musa acuminata
ggctatatataagtagcaacgtggagtgacgagtgggaatagacaaaggaaatggcgtgctccttcccggtcctcgacttggagaagctccgtggagaggagagagagcagtccatggacctccttcgtgacgcttgcgagaaatggggcttctttgaggtgctctcattcttttacgtgaacaagttgatgcccacgagcatttgaagctaattatcatggctttccatttgcttgctgatgtagctgctcaaccatgggatctcgcatgagctgatggacgaggtggagaggcggaccaaagcgcactacgagcaatgcaggaagcaaaagttcaaacagttggcgtgcaaggctctcaagagcggacccgggacggatgtcaccgacatggactgggagagcaccttcttcctgcgccatctccccgtctccaacatgtccgacttcccagacatggacgaggagtaccggtaccgcttcgatttccttcgttacagcgcaccccccaccaccatcgactgtagtctgccccgactaaccttcgccttcaggaaggcgatgacggaattcgcgacggggttagagaagctggcggagcgtcttctcgatctgctctgcgagaacctcggcctggaggagggttacctcaagaacgccttctacggatccaaaggtccgaactttggcaccaaggtgagcaactacccgccatgccctcgcccggagctgatccacggcctgcgagcccacaccgacgccggcggcatcatcttgctcttccaagacgaccgcgtcagcggcctccagcttctcaaggatggccagtggatcgacgtgccgcccatgcaccactccatcgtggtcaacctcggagatcagatagaggtcctctctctctctctctctctctctctttctctctctgcgggacaaaagatcaaacaacatgagggcgttcatgcaggtgatcacgaacggcaagtacaagagcgtgctgcaccgggtggtggctcggagcgacggcaacaggatgtcgatcgcctccttctacaacccgagcggcgacgccgtcatctaccccgcgccctccctggtccagaaggaagcggaggcgtacccgaggtttgtgttcgaggactatatgaagctctacgtcacgcaaaagtttcaagcgaagcagcccaggtttgaagcaatgaaggccacggtaacagtcaatggccaacctacgcctacaccttaggacaccacgacgtctcacgtggagatgccaccatctattagaatgtggcatccaattgtggaaataataagcgaagcactatgaacgtggctttttttagtctcgagggttatgtcgtcgatccaattttccacttct

SEQ ID NO: 47 - DNA, 1402 bp, Musa acuminata
acactccagatagaaagcacaagtgcaatcagggaagaaagagcgtgtcatggattcctttccggttatcgacatggagaagcttttgggaagggagagaggagaagccatggagatcctccgagatgcttgcgagaaatggggcttctttgaggtgctgaagcatacataactggttttgcttctttgaactatatatatattgctaaaatgtactatttgcacatgcaatctgtgtgtagattttaaaccatggcatctcacatgacctcatggatgaagtggagaaggtgaacaaagaccagtacaacaaatgcagggagcaaaagttcaacgagttcgccaacaaagcactggaaaacgccgactcagaaatcgaccacctcgactgggaaagcacctttttcctgcgtcatctccccgtctccaacatttctgagatccccgatcttgatgaccagtataggttgcacgatctgatcatgatgtcatcttctggcctggtcttttcaccttgctcatcgtttcgtttcttgggacgatgactgcgtgcaggaaggcgatgaaggaatttgcggcagagatggagaagctggcagagcggctgctcgacttgctgggtgagaacctggggctggagaaggggtacctgaagaaagccttctctaatggatccaaggggccaacctttgggaccaaggtcagcagctacccgccatgcccgcgcccggacctggtgaagggcctgagggcgcacaccgacgccggaggcatcatcttgctcttccaggacgaccaggtcagcggcctgcagttcctcaaggacggcgagtggctggacgtgccccccatgcgccacgccatcgtcgtcaacctcggcgaccagctcgaggtttgggtcctctttgctctcgtttccgctgcccgtcgtctgtgatgttgaatgcaacgaggtctgcaggtaatcaccaatggcaagtacaagagcgtggtgcaccgcgtggtggctcagactgatggcaacaggatgtcgattgcctccttctacaaccccgggagcgacgctgtgatcttcccggcccccgctcttgtggagaaggaagcggaggagaagaaggaggtctatccgaagttcgtgttcgaggattacatgaagctctacgtcgggcataagttccaggccaaggagccaagattcgaagccatgaaagccatggaagcagttgccacccacccaatcgctacctcttaagtgacagcccccaagttagtgcatgtcgctgtacttcgcgttaggaagctgtcgtctatgtctatgtaacccgatggatgtgtggtatgtacgtgtgtgagccttttctaatgaagcaaatcatataatatatatatatatatatatata

SEQ ID NO: 48 - DNA, 1761 bp, Musa acuminata
atggccggccttccttattcagctcctcaccctgccaccatctccgcttcctccaactcctttgcatgccccttccgcagcaaggggcttgtcttcccctaccctaccagaagagcactccatgttcgtcccaacatcgcatgcaaggcaggcgaggagcacgagatcgctgctaaggtcgaccgacgcgacgtactcgtgggcctcggtgggctctgcggagccgccgctggccttggcgggttcgataaagccgccctcgctaaccccattcaggcccctgatctctccaagtgcggccctgccgacctccccaccggcgtgccagtcgtcaactgctgcccgccctaccgtcccggtgcgaagattgtggatttcaagcggccgtcgccgtcctccccactccgcgtccgccccgccgcccacttggttgaccccgagtacctggccaagtacaagaaggccatcgagctcatgaaggcgctcccggccgatgaccctcgcaacttcatgcagcaggccgacgtccactgcgcctactgcgacggcgcttacgaccagatcggcttccccaaccttgagatccaagtccacaacagctggctcttcttcccctggcaccgcttgtacctctacttcaacgagaggatcctcggcaagctcatcggcgacgacaccttctcgctccctttctggaactgggacgcacccggcggaatgatgctgccttcgatctacgccgatccttcgtcacccctctacgacaaacttcgcgacgccaagcaccaacctcctgtccttgtcgacctcgactacaatggaaccgacccaaccttccccgacgcccagcaaatcgatcacaacctcaagatcatgtaccgccaagtcttctccaacggcaagacgccgttgctgttcttaggctcagcttaccgtgccggtgaacagcctaaccctggcgcgggctccgtcgagaacatgccgcacaacaacgtgcacttgtggaccggcgaccgcacccagcccaacttcgagaacatgggcaccttctacgccgcggcgcgcgaccccatcttcttcgcccaccacgccaacatcgaccgcatgtggtacctgtggaagaagctcagcaggaagcaccaggacttcaatgactcggactggctcaaagcttccttccttttctacgacgagaacgccgacttagttcgggtcacggtcaaggactgcttggagaccgattggctgcgctacacgtaccaagacgtgaagatcccatgggtgaacgcccgaccgactcccaagctcgccaaggcgaggaaagccgccagcagttcgctgaaagccaccgcggaggtgcagttccctgtgacgctggaatccccggtcaaagcgacggtgaagaggcccaaggtggggaggagcggcaaggagaaggaagatgaggaggagatactcatagtggaggggatcgagttcgaccgcgactacttcatcaagttcgacgtcttcgtgaacgcgacggagggcgacggcatcacggccggggccagcgagttcgccggcagcttcgtgaacgtcccgcacaagcacaagcaccgcaaggatgagaataagctgaagacgaggctgtgtcttggaatcaccgacctgctcgaggacatcggcgcggaggacgacgacagcgtgctcgtcaccatcgtgccgaaggcgggcaaaggaaaggtgtccgtcggcggtcttcggattgacttttccaag

SEQ ID NO: 49 - DNA, 1734 bp, Musa acuminata
atggcttctatctcgcagctaatcactacaagcatccccaccaccttttctctctcctattcatgccccttccctccgagaaccacggtatcgatctccggcttcaaacacctccaccacgtttcccccatctcatgctccaccagagaccacaaccaacccctcgtcgatcgccgccacgtcctcgtcggaataggcagcctctacggcgcctccgctgcactaacgtccctccgcgaggccagtgcggcaccgatcgcggcgcccgacctgtccgcatgcgggctggctgacctccctccggatgccactccgacgaactgctgcccgccgtccgcgggagacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttcttccagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcagtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggctgatgggagcatcatggtgactttggtgccgaggcaggggaaggggaaggtgaaggttggcagtctcaagatcgagttagttgat

SEQ ID NO: 50 - DNA, 1608 bp, Musa acuminata
atggcagagatcggcaatccaaatgaaaacaacacctcaatatttgccgaccgcaagccgccgaaacgcctcgtgttgccattcggcgtccgaccaccagtggcagaggcactgacaaaggttgatgcaagtttctgcgaccccaagaacaacaatgagtgtataaacttaggatcccaaggaggcgagtgcgccgccaactgctgccttcccatccctcccggtgcgaagattgtggatttcaagcggccatcgcggtccacccccctccgcgtccgccccgccgcccacttggtcgaccccgagtacctggccaagtacaagaaggccatcgagctcatgaaggcgctcccggccgacgaccctcgcaacttcatgcagcagtccaacgtccactgcgttcactgcgacagcatccccgacaatgacatccaagtccaccagaactggttcttctacccctggcaccgctggtacctctacttcaacgagaggatcctcggcaagctcatcggcgacgacaacttcacgctccctttctggaactgggactcgctcggcggaatgatgctgccgtcgatctacgccgacccttcgtcgcccctctacgacaaccttcgcgacgccaagcaccaacctcctttccttgtcgacttcgacttcaatggaaccgacccaggcttcaccgacgcccagcaaatcgatcacaacctcaagatcatgtaccgccaattcttctccaacggcaagaagccgatgctgttcttaggctcaccttaccgcgggggcgacaagcctaaccccggcgggggctccgtcgagaacacgccgcacaacaacgtgcacacgtggaccggcgaccgtactcgtcccaacttcgaggacatgggcaccttctactcggcggggcgcgaccccatcttcttcgcccaccacgccaacatcgatcgcatgtggtccctgtggaagaagctcagccgtaagcaccgggacttcaatgactcggactggctgaaaacttccttcctcttctacgacgagaacgccgacttagttcgggtcacggtcaaggactgcttcaagacccggtggctgcgctacaagtaccaagacgtggagatcccatgggtgaaagcccgaccgactcccaagctcaccaaggcgaggaaagccgccagcggatcgctgaaacccaccgcggaggcgcagttccctgtgacgctggaatccccggtcagcgcgacgctgaagaggcccaaggtggggaggagccgcaaggagaaggaagaggaggaggaagtgctcatagtggaggggatcgagttcgaccgcgaccagttcatcaagttcgacgtcatcgtgaacgcgacggagggcgatggcatcacgcccgcggacagcgagttcgccggcagcttcgtgaacaccccgcacaggcacaggcacctcaaggaggagaacaaggggacgacgaggctgtgtctggggatcaccgacctgctcgaggacatcggcggggaggccgacgacggcgtgctcgtcaccatcgtacccaaggcaggcaagggcaaggtttccgtcggcggtcttcggattgacttcaccaag

SEQ ID NO: 51 - DNA, 1770 bp, Musa acuminata
atgtccctgctgttgaactctagcttcaccggtgcttcctctgcatgcctcctccaacgggaaaggtcccgccgccgccgcctccacgtccctggcgtgacatgccgccagggcagtaatggtgaccgcagcgatgccgcccgccagcagcagtcgccgccgctgctggatcggcgcgacatgctgttgggtttaggagggctttacggcgtgaccgcaggacccaaggttctggcggcgccgataatgccgccggatctgtccaagtgctaccctgccaccgcacctgccctcgacaacaaatgctgcccgccttacgaccccggcgagacgatctcggagtacagcttccccgctacgcccctccgggtgcggcggccggcccatatcgtgaaggacgatcaggagtatatggacaagtacaaggaggcagtgaggaggatgaagaatctgccggcagaccacccttggaactactaccagcaggcgaacatccactgccagtattgcaactacgcctaccaccagcaaaataccgacgacgtgcccatccaggtccacttcagctggatcttcctcccatggcaccgctactacctccacttctacgaaaggatcctcggcaagctcatcgacgacgacaccttcaccatcccattctggaactgggacaccaaggacggcatgacgttccccgccatcttccaggatgcggcatccccgctgtacgacccgaaacgcgaccaacgccacgtcaaggacggcaagatcctcgacctcaagtacgcctacaccgaaaacactgcatccgacagcgagatcatacgggagaacctctgcttcatacagaagacgttcaagcacagcctgtcgctggcggaactgttcatgggggatcccgtgcgcgcgggggagaaggagatccaggaggctaatgggcagatggaagtcatccacaatgcggcgcacatgtgggtcggagagccggacggatacaaggaaaacatgggggacttctccaccgccgcccgcgattctgttttcttctgccaccattgcaatgtcgaccgcatgtgggacatctaccgcaacctccgcggcaaccgcgtcgagttcgaagacaacgactggttggacagcaccttcctcttccacgacgagaacgaacagctcgtcaaagtcaagatgagcgactgcctcaacccgaccaagcttcggtacacgttcgaacaagttcccctcccatggctgggcaaaatcaattgccagaagacggcagagacgaagtccaaggccacgacggagctgtcgctgacgcgcgtgaacgaattcgggacgacggcccaggcactcgacgcgagcaacccgctgcgggtgatcgtggcaaggccgaagaagaaccgcaagaagaaggagaagcaagagaaggtggaggtgattcagatcaaggatattcaggtgaccaccaacgagacagctcgcttcgacgtctacgtcgcggttccttacggtgacctcgccggacccgactacggcgagttcgcgggcagctacgtgaggctggcgcataggatgaagggaagcgacgggaccgcagagcaggtccccaagaagaagggaaagctcaagctgggtattacgccgctgctcgaggacatcgatgctgaggacgccgacaagttggtggtcaccctggttctccgcaccgggagcgtcaccgtgggcggagtttccatcaatctcctgcagacagattctaccgccgccatc

SEQ ID NO: 52 - DNA, 1524 bp, Musa acuminata
atgtccctgctgttgaactctagcttcaccggtgcttcctctgcatgcctcctccaacgggaaaggtcccgccgccgccgcctccacgtccctggcgtgacatgccgccagggcagtaatggtgaccgcagcgatgccgcccgccagcagcagtcgccgctgctgctggatcggcgcgacatgctgttgggtttaggagggctttacggcgtgaccgcaggacccaaggttctggcggcgccgataatgccgccggatctgtccaagtgctaccctgccaccgcacctgccctcgacaacaaatgctgcccgccttacgtccccggcgagacgatctcggagtacagcttccctgctacgcccctccgggtgcggcggccggcccatatcgtgaaggacgatcaggagtatatggacaagtacaaggaggcagtgaggaggatgaagaatctgccggcagaccacccttggaactactaccagcaggcgaacatccactgccagtattgcaactacgcctaccaccagcaaaatgccgacgacgtgcccatccaggtccacttcagctggatcttcctcccatggcaccgctactacctccacttctacgaaaggatcctcggcaagctcatcgacgacgacaccttcaccatccccttctggaactgggacaccaaggacggcatgacgttccccgccatcttccaggatgcggcatccccgctgtacgacccgaaacgcgaccaacgccacgtcaaggacggcaagatcctcgacctcaagtacgccatcaccgaaaatgaaagcactgcatccgacagcgagatcatacgggagaacctctgcttcatacagaagacgttcaagcacagcctgtcgctggcggagctgttcatgggggatcccgtgcgcgcgggggagaaggagatccaggaggctaacgggcagctggaagtcatccacaatgcggtgcacagttgggtcggagagccgagcggaaactatgaagacatgggcgacttctccaccgccgcccgcgattctgttttcttctgccaccattgcaatgtcgaccgcatgtgggacatctaccgcaacctccgcggcaaccgcgtcgagttcgaagacaacgactggttggacagcaccttcctcttccacgacgagaacgagcagctcgtcaaagtcaagatgagggactgcctcaacccgaccaagcttcggtacacgttcgagcaagttcccctcccatggctgggcaaaatcaattgccagaagacggcagagacgaagtccaaggccacgacggagctgtcgctgaatcgcgtgaacgaattcggaacgacggcccaggcactcgacgcgagcaacccgctgcgggtgatcgtggcaaggccgaagaagaaccgcaagaagaaggagaagcaagagaaggtggaggtgattcagatcaaggatattaaggtgaccaccaacgagacagctcgcttcgacgtctacgtcgcggtcccttacggtgacctc

SEQ ID NO: 53 - DNA, 1776 bp, Musa acuminata
atgtctctcctgttgaactctagcctcaccggagcttcctctgcatgcctcctccgtcgagaaaagtgccgccgccgcggccgcggtcacgtccacggcgtgacatgccaccaggggggtaatgatgaccgcagagaggccgcccggcagcagcggtcccggttgctgctggatcggcgcgacatgctgttgggggggttaggagggctttacggcgtgaccgcagggcccaaagttctggcggagccgataatgccgcctgatctgtccaagtgccacgatgccaacgcacctgccctcgacaaccactgctgcccgccttacagtggcagcgagacgatcttggagtacgacttccccgctacgcccctccgggtgcggcgaccggcccacctcgtgaaggatgatcaggagtatatggacaagtacaaggaggccgtgaggaggatgaagaacctgccggcagaacacccttggaactactaccagcaggcgaacatccactgccagtattgcaacgacgcctactaccagcaaaataccgacgacgtgcccgtccaggtccacttcagctggatcttcctcccctggcaccgctactacctccacttctacgagcggatcctcggcaagctcatcgacgacgacaccttcaccatccccttctggaactgggacaccaaggacggcatgacgttccccgccatcttccaggatgcggcatccccgctgtacgacccgaaacgcgaccaacgtcacgtcaaggacggcgcgatcctcgacctcaagtacgcctacaccgaaaacactgcatccgacagcgagatcatacgggagaacctctgcttcatacaaaagacgttcaagcacagcctgtcgctggcggagctgttcatgggggatcccgtgcgcgcgggggagaaggagatccaggaggcaaacgggcagctggaagtcatccacaatgcggcgcacatgtgggtcggagagccggacggatacaaggaaaacatgggcgacttctccaccgcggcccgcgattctgttttcttctgccaccattccaatgtcgaccgcatgtgggacatctaccgcaacctccgcggcaacagcgtcgagttcaacgacaaagactggttgcacagcaccttcctcttccatgacgagaacgagcagctcgtcaaagtcaagatccaggactgccttaacccgaccaagcttcggtacacgttcgagcaagttcccctcccatggctgggcaatataaattgccagaagacggcagagacgaagtccaagtccacggcagagctgtcgctgaagcgggtgggcgaattcgggacgacacccaaggcgctcgacgcgagcaacccgctgcgggtgatcgtggcaaggccgaagaagaaccgcaagaagaaggagaagcaagagaaggtggaggtgctccagatcaaggatattaaggtgaccaccaacgagacagctcgcttcgacgtctacgtcgccgttccttacggtgacctcgccggacccgactacggcgagttcgtgggcagcttcgttaggctggcgcataggaagaagggaagcgacgggaccgaagagcagggccccaagaagaagggaaagctcaagctgggtattacggcgctgctcgaggacatcgatgctgaggacgccgacaagttggtggtcaccctggttctccgcaccgggagcgtcactgtgggtggagtttccatcaaactcctgcagacagatactcccgccgtcatc

SEQ ID NO: 54 - DNA, 1512 bp, Musa acuminata
atggctctccagttgaactctagcttcaccggagcttcctctgcatgcctcctccatcgggaaaggtcccgccgcctcaacgtccctgtcgtgacatgccgccaggggaataatgatgatcgcagcgatgccgctcgccagcagaaatccccgtcactactggatcggcgcgacatgctgctggggttaggagggctttatggcttgaccgcaggacccaaagttctggcgaagccgataatgccgcctgatctgtccaagtgccacgatgccaaggcacctgccctcgacaaccactgctgcccgccttacaaccccagcgagacgatctcggagtacggcttccccgctacgcccctccgggtgcggcggccggcccacctcgtgaaggacgatcaggagtatttggacaagtacaaggaggccgtgaggaggatgaagaatctgccggcagaccacccttggaactactaccagcaggcgaacgtccactgccagtactgcaactacgcctactaccagcaaaataccgacgacgtgcccgtccaggtccacttcagctggatcttcctcccctggcaccgctactacctccacttctacgagcggatcctcggcaagctcatcgacgacgacaccttcaccatccccttctggaactgggacaccaaggacggcatgacgttccccgccatcttccacgaagagtcatccccgctgtctgacacgaaacgcgaccaacgccacgtcaaggatggcaagatcgtcgacctcaagtacgcctacaccgaaaaccctgcctccaacagcgagatcattcgagagaacctctgcttcatacagaagacgttcaagcacagcctgtcgctggcggagctgttcatgggggatcccgtgcgcgcgggggagaaggagatccaggaggctaacgggcagctggaagtcatccacaatgcggtgcacatgtgggtcggagagccgtgcggatacaaggaaaacatgggcgatttctctaccgccgcccgcgattctgttttcttcagccaccactccaatgtcgaccgcttgtgggaaatctaccggaacctccgcggtaaccgcattgagttcgaagacaacgactggttggacagtaccttcctcttctacgacgagaacgagaagctcgtcaaagtcaagatgggggactgcctcaacccgaccaagcttcggtatacgttcgagcaagttcctctcccatggctgggcaaaattaattgccagaagacgacagagacgaagtccaagtccacgacagagatgtcgctgacgcgcgtgggagaattcgggacgacgcccaaggcgctcgacgcgagcaacccgctgcgggtgatcgtggcaaggccgaaaaagaaccgcaagaagaaggagaagcaagagaaggtggaggtgcttcagatcaatgatattaaggtgaccaccaacgagacagctcgcttcgacgtctacgtcacggttccttacggtgacctc

SEQ ID NO: 55 - DNA, 1770 bp, Musa acuminata
atggtcagccttcctaaagctactcttcctctctcctccctctcccctccctccaactccaactccaactccaactccaactcctttgcatgcgccttccatttttcttaccctgatagaagacgccatgcccactccaagatctcatgcaaagctagcgatgagcatgagatgactgcaaatgccaagctcgaccgccgcgatgtgctcgttggcctcggcgggctttgtggagccgctgccggcctcgggatcgacggtaaggccctcggaaatcccatccaggcgcctgatcttaccaagtgcggccccgccgatctacccaaaggtgcaacgcccaccaactgctgcccgccttattttcccgacaagaagattatcgatttcaagcgtccgccgaattcgtcacccctccgtgtccgcccggccgcccacttggtcgactccgactacctggacaagtataagaaggcggtggagctcatgagagcactgccggccgacgatccgcgcaacttcatgcagcaggccaatgtccactgcgcttactgtgatggcgcctacgaccagatcggcttccccaacctcgagctccaagtccacaactcctggctcttcttcccttggcaccgcttctacctctacttccacgagaggatcctcggcaagctcataggcgacgacactttcgcccttcctttctggaactgggacgcgcccggcggcatgaagctgccgtcgatctacgccgatccttcgtcctcgctctatgacaagtttcgcgacgccaagcaccagccgccggtcctcgtcgacctcgactacaacggaaccgaccctagtttcaccgacgcagagcagatcgatcagaacctcaagatcatgtaccggcaggtgatctccaacggcaagacgccgttgctgttcttaggctcggcttaccgtgccggcgacaacccaaaccccggcgcgggctcgctcgagaacataccacacggccccgtccacgggtggactggcgacagaaaccaacccaatctcgaggacatgggcaacttctactccgcggggcgcgaccctatcttcttcgcccaccattcaaacgtcgaccgcatgtggtacttgtggaagaagctcggcgggaagcatcaggactttaacgataaggactggctcaacaccaccttcctcttctacgacgagaatgctgacttagttcgagtcaccctcaaggactgcttgcagccggagtggcttcgttacgattaccaagacgtcgagatcccgtggctgaagacccggccgactcccaaagccttgaaggcgcagaaaaccgcagcgaaaacactgaaagctacagcagagacgccgttcccggtgacgctgcaatccgcggtgagcacgacggtgaggaggcccaaggtatcgaggagcggcaaggagaaggaagaggaagaggaggtcctcatcgtggaggggatcgagttcgaccgcgactacttcataaagttcgacgtcttcgtgaacgccaccgagggtgagggcatcacgccgggcgccagcgagttcgcgggcagcttcgtcaacgtcccgcacaagcacaagcacagcaagaaggagaagaagctgaagacgaggctctgcctggggatcactgacctgctcgaggacatcggggcggaggacgacgacagcgtgctcgtcaccatcgtcccgaaagccggaaagggcaaggtgtcggtcgccggcctccgcatcgatttcccaaat

SEQ ID NO: 56 - DNA, 1722 bp, Musa acuminata
atggagggcaaacgatggttgtctcttctcctcctcgtgcttgttctcgtgggcatctccatggatctcccgagagaagctccggcggcttcttctaatatcttgaagagttcatctgccagaataccagtgaatcctcaaggcggagaacagagagatggaagcaagagcaagggcattcccctcaaagcaaacctatcggtttgccatgcttcattctcggacgccgatcgtccggtctactgttgcccggcctggaaagacgccgaccaaaccttgctcgacttcgagttcccggatccgtcgtcgccggtgcgtatccgacggcctgcccatctcgtcgacgaggagttcgtggccaagtatgagagggcggtggccatcatgaagcagatcccgcctgaccatccccacaacttttggcgccaggccaacatgcactgcctctactgcaccggcgcctacgaccagatgaactcctctgccctcttcaagatccacaggtcatggctcttcttcccctggcaccgagccttcatctatttccacgagcgcatcctcgggaagttcatgggagacgacaccttcgcgctcccctactggagctgggacacccccgagggcatgtggttccccgacatctaccggaagggagctctgaatgagacggagcgcgacgccattcacctacgggaggccgccgtcgatgacttcgactacgtggatcatgacctcgccagcgacgtgcagatcgccgacaacctcgcgttcatgtaccaccagatgatctcgggagcgaagaagaccgagctgttcatgggttgcaagctgcggtccggcgtcgaggggtggtgtgatgggcccgggacgatcgaagcggcacctcacaacacgttgcacagctgggtagggaacaggtacaaccccgaaagagagaacatgggggcgttctactccgccgcgcgagacgaagtgttcttcgcgcaccactccaacatcgaccgcatgtggaccgtgtggaagaagctgcacggcgacaagccggagttcgtcgaccaggagtggctcgagtcggagttcaccttctacgacgagaatgtgcgcctgcgcaggatcaaggtgcgcgacgtgttgaacatagacaaactcaggtaccggtacgaagacatcgacatgccatggctcgctgcacgtcccaagccttccgttcaccctaagatcgcgcgcgacatattgaagaagcgcaatggcgaaggcgtactgagaatgcccggcgaaacggatcgttcacaactctccgaatatggtagctggacactggacaagaccatcaccgtgagggttgacaggccaaggatcaacaggacagggcaagaaaaagaggaagaagaggagatcttattggtctacggaatcgatactaagagaagcagattcgtcaaattcgatgtgttcatcaacgtcgtcgacgaaaccgtgctgagcccaaagtcgagggagttcgcagggaccttcgtcaatctccaccacgtctcgaggacgaaaagccatgacgatggcggcatggattcgaagatgaaaagccaccttaagctcggtatatcggaacttttggaagacctcgaggcagacgaagatgacagcatctgggtgacactggtgccaagaggcggcacgggggtcaacaccaccgtggacggcgtccggatcgactacatgaag

SEQ ID NO: 57 - DNA, 39 bp, Artificial Sequence
  ALS1 Ma06_g18100
gtcgccatcaccggccaggtccctcgccgcatgatcggc

SEQ ID NO: 58 - protein, 13 aa, Artificial Sequence
  ALS1 Ma06_g18100
VAITGQVPRRMIG

SEQ ID NO: 59 - DNA, 39 bp, Artificial Sequence
  ALS2 Ma10_g11980
gtcgccatcaccggccaggttccccgccgcatgatcggc

(19) United States
(12) Patent Application Publication
(10) Pub. No.: US 2025/0034586 A1
(43) Pub. Date: Jan. 30, 2025

(54) METHOD FOR EDITING BANANA GENES

(71) Applicant: Tropic Biosciences UK Limited, Norwich, Norfolk (GB)

(72) Inventors: Marta Daniela de Sa MENDES, Norwich, Norfolk (GB); Robert Tristan GREEN, Norwich, Norfolk (GB); Ofir MEIR, Norwich, Norfolk (GB); Cristina PIGNOCCHI, Norwich, Norfolk (GB)

(73) Assignee: Tropic Biosciences UK Limited, Norwich, Norfolk (GB)

(21) Appl. No.: 18/575,208

(22) PCT Filed: Jun. 30, 2022
(86) PCT No.: PCT/EP2022/068060

§ 371 Date: Dec. 28, 2023

(30) Foreign Application Priority Data
Jul. 2, 2021 (GB) 2109586.4

(51) Int. Cl. C12N 15/82; C12N 9/22; C12N 9/78; C12N 15/11

(52) U.S. Cl. CPC C12N 15/8274; C12N 9/22; C12N 9/78; C12N 15/111; C12N 15/8213; C12Y 305/04002; C12Y 305/04005; C07K 2319/00; C12N 2310/20

(57) ABSTRACT

The present invention provides improved methods for generating banana embryos and plants carrying desired mutations in target sequences, comprising introducing at least one mutation in at least one endogenous acetolactate synthase (ALS) gene. The invention also provides banana plants and products generated by such methods.

### CROSS REFERENCE TO RELATED APPLICATIONS

This application is a national stage application under 35 U.S.C. § 371 of International Application No. PCT/EP2022/068060, filed internationally on Jun. 30, 2022, which claims the benefit of priority of Great Britain Application No. 2109586.4, filed on Jul. 2, 2021, the contents of each of which are hereby incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 251502013300seglist.txt, date recorded: Dec. 20, 2023, size: 113,607 bytes).

### FIELD OF THE INVENTION

The present invention relates to improved methods for generating banana embryos and plants comprising desired mutations in target sequences, and banana plants and products generated by such methods.

### BACKGROUND OF THE INVENTION

Modern gene editing technologies provide significant opportunities for generating improved crops with, for example, greater pest resistance and desirable traits. Gene editing presents the possibility of generating improved non-transgenic crops that do not contain any heterologous sequences from other species, which is preferred by consumers and regulators. Prior to the development of modem gene editing techniques, improvement of non-sexual crops such as banana was attempted using techniques involving transformation with large plasmids carrying heterologous promoter and marker sequences derived from other species (for example in Ganapathi et al., Plant Cell Reports, 2001, 20:157-162). However, integrating heterologous sequences from other species is incompatible with the generation of non-transgenic plants and introduced promoters and genes may have unpredictable effects that are very different from the effects of even related endogenous genes.

Non-transgenic genome editing using technology such as CRISPR in banana presents several challenges. This crop is produced via non-sexual propagation, and modern varieties of banana (in particular the major export variety “Cavendish”) are parthenocarpic; they produce seedless fruits. Therefore, it is not possible to segregate out any introduced genetic sequences, such as selectable markers or gene editing machinery, as would be performed with sexually reproductive crop species, to provide non-transgenic edited plant lines. Instead, to achieve non-transgenic genome editing in banana, editing must occur without stable integration of transgenes into the genome. However, this makes it very difficult to identify banana plants carrying the desired edits. It is possible to genotype thousands of plants to identify edited lines that lack any of the transformation plasmid, but this is very labour intensive, time consuming and expensive.

Various methods for introducing mutations into different crop species have been proposed, such as in WO 2018/202199. However, the processes for transforming and developing banana plants are very different to those used for sexually reproductive cereal crops such as wheat and corn.

There is a requirement for improved methods of editing genes in banana.

### SUMMARY OF THE INVENTION

The inventors have developed an improved method for generating banana embryos and plants comprising desired mutations in target sequences. The method utilises co-editing of a target sequence and endogenous acetolactate synthase (ALS) genes in combination with ALS inhibitor selection to provide a highly efficient and effective strategy for editing banana genes.

The ALS genes encode proteins that function in the branched-chain amino acid biosynthetic pathway. ALS proteins are irreversibly competitively inhibited by several herbicides from the sulfonylurea family. These compounds are potent herbicides and are used as effective broad-spectrum weed killers. Several ALS amino acid substitutions have been identified, both naturally occurring and artificially generated, that increase tolerance to sulfonylurea compounds. These are typically all amino acid residues that form the active site pocket and alter the binding potential of the herbicide compound.

In the methods of the invention, an agent such as a base editor is used to edit a banana ALS gene or genes, for example to substitute a proline for a serine in the peptide sequence(s), and such substitutions confer resistance to ALS inhibitors such as the sulfonylurea compound chlorsulfuron. Banana cells, embryos and plants containing these precise edits survive treatment with ALS inhibitor, whereas wild type plants or embryos are killed. In parallel, a target sequence or sequences is/are also edited (“co-edited”), for example by targeting the target sequence with the agent. Transforming banana cells in this manner leads to plant material that can be treated with ALS inhibitors, selecting for the presence of the ALS edit, and significantly enriching the population for the presence of the desired mutation in the target sequence.

Therefore, in a first aspect, the invention provides a method of generating at least one banana embryo or plant comprising at least one desired mutation in a target sequence or sequences, the method comprising:
a. contacting a banana cell with at least one agent operable to introduce the at least one desired mutation in the target sequence(s) and at least one agent operable to introduce at least one mutation in at least one endogenous acetolactate synthase (ALS) gene so as to provide resistance to an ALS inhibitor,
b. optionally culturing the banana cell to generate a banana cell population, tissue, or plant;
c. treating the banana cell, cell population, tissue or plant with an ALS inhibitor to select at least one banana cell, tissue or plant mutated in at least one endogenous acetolactate synthase gene and comprising the desired mutation(s) in the target sequence(s); and
d. generating at least one banana embryo or plant from the banana cell, tissue or plant selected in step (c).

The invention also provides a method of generating at least one banana embryo or plant comprising a desired mutation in a target sequence, the method comprising:
a. contacting a banana cell with at least one agent operable to introduce the desired mutation in the target sequence and at least one agent operable to introduce a mutation in an endogenous acetolactate synthase (ALS) gene that provides resistance to an ALS inhibitor;
b. optionally culturing the banana cell to generate a banana cell population, tissue, or plant;
c. treating the banana cell, cell population, tissue or plant with an ALS inhibitor to select at least one banana cell, tissue or plant comprising the mutation in the endogenous acetolactate synthase gene and comprising the desired mutation in the target sequence; and
d. generating at least one banana embryo or plant from the banana cell, tissue or plant selected in step (c).

The examples demonstrate that the methods of the invention provide a population of banana cells, embryos or banana plants that are enriched for the desired mutation(s) in the target sequence(s). Strikingly, the examples demonstrate that introducing mutations into endogenous ALS genes in banana is effective for providing resistance to ALS inhibitors and therefore selection with ALS inhibitors can be used to enrich for banana embryos and plants that are also edited at a target sequence. The examples demonstrate that it is not necessary to incorporate heterologous ALS sequences or drive expression of ALS sequences from a strong promoter in order to provide resistance to an ALS inhibitor in banana. The methods of the invention are particularly useful because bananas are asexual and it is impossible to breed bananas to achieve improved traits or phenotypes, and it is impossible to breed out any introduced sequences.

Unlike crops such as wheat, banana comprises two endogenous ALS genes—ALS1 and ALS2. Strikingly, the examples show that introducing mutations into either ALS1 or ALS2, and at either one, two or three alleles (banana is triploid), is effective for providing resistance to an ALS inhibitor, and the examples demonstrate that it is not necessary for all endogenous ALS genes and alleles to be mutated. Therefore, the method of the invention is effective for generating and selecting banana embryos or plants comprising the desired mutation(s) in the target sequence(s), even if the transformation and editing process is relatively inefficient, because in banana mutations at either ALS1 or ALS2 and at single alleles can provide resistance to an ALS inhibitor.

In preferred embodiments, the banana cell is a protoplast or an embryogenic cell, such as a protoplast in a protoplast cell suspension or an embryogenic cell in an embryogenic cell suspension. In such methods step (a) comprises contacting the protoplast cell suspension or embryogenic cell suspension. The examples demonstrate that such cells and suspensions can be efficiently and effectively contacted with agents such as base editors and are amenable to simultaneous editing of an endogenous ALS gene and a target sequence.

In preferred embodiments, the method utilises base editors and the agents operable to introduce the mutations comprise a base editor and guide RNAs or comprise nucleic acid constructs that encode a base editor and guide RNAs. Base editors are particularly suitable for simultaneous editing of an endogenous ALS gene and a target sequence, without requiring the incorporation of any heterologous DNA, as demonstrated in the examples. Particularly preferred base editors are discussed further below.

In certain embodiments, the method comprises introducing a mutation in the endogenous ALS1 gene. The inventors have identified that ALS1 is expressed at a higher level than ALS2 in banana, so mutating the endogenous ALS1 gene may provide increased resistance to an ALS inhibitor. In certain embodiments, the method does not comprise introducing a mutation in the endogenous ALS2 gene, and for example, the agent does not comprise any sequence such as a guide RNA targeted to the endogenous ALS2 gene.

In certain embodiments, the method comprises introducing a mutation in the endogenous ALS2 gene. The inventors have identified that ALS2 is expressed at a lower level than ALS1 in banana, so it is surprising that mutation of ALS2 is adequate to provide resistance to an ALS inhibitor and allow selection of successfully edited banana embryos or plants. Mutating the endogenous ALS2 gene may allow for the improved selection of embryos or plants that are edited at multiple alleles of the ALS2 gene and multiple alleles of the target sequence(s), because the lower level of expression of endogenous ALS2 means that embryos and plants with single edits may have limited resistance to ALS inhibitors, which could allow increasing ALS inhibitor treatment to be used to select for multiple edits. Also, mutating the endogenous ALS2 gene may provide improved selection for generating non-transgenic, trait edited plants. Also, since ALS2 is expressed at a lower level compared to ALS1, it could mean ALS1 is the main source for biosynthesis branched amino acids, and so mutating ALS2 will disturb the cell homeostasis to a lesser extent. In certain embodiments, the method does not comprise introducing a mutation in the endogenous ALS1 gene, and for example, the agent does not comprise any sequence such as a guide RNA targeted to the endogenous ALS1 gene.

In certain embodiments, the method comprises introducing mutations in both the endogenous ALS1 gene and the endogenous ALS2 gene. Such methods may allow more aggressive selection with ALS inhibitors to be used, to generate populations of embryos and plants that are more highly enriched for the desired mutation in the target sequence.

In preferred embodiments, step (c) comprises incubating an embryogenic cell suspension or culturing an embryo in the presence of an ALS inhibitor. The examples demonstrate that selection with an ALS inhibitor at the embryogenic cell suspension or embryo stage is possible in banana, which can provide a faster and more efficient process because it is not necessary to wait until plantlets develop. ALS is a chloroplast-localised enzyme and embryogenic cell suspensions and embryos are non-green tissues, so it is surprising that selection with ALS inhibitors is effective at the embryogenic cell suspension or embryo stage. In certain embodiments, the method does not comprise treating a plantlet or plant with an ALS inhibitor. ALS inhibitor treatment at the embryo stage and also the plantlet or plant stage may unnecessarily inhibit growth and development without improving selection of co-edited plants, for example because mutation of only one ALS gene at one or two alleles may not provide functional tolerance to ALS inhibitors (as might be provided with over-expression of a heterologous ALS sequence driven by a promoter), so co-edited plants might still be vulnerable to continued ALS treatment. In preferred embodiments, the method comprises treating a banana cell with an ALS inhibitor and selecting at least one banana cell, such as an embryo, comprising a mutation in an endogenous acetolactate synthase gene and comprising a desired mutation in a target sequence. In certain embodiments, the method comprises selecting at least one banana cell, such as an embryo, following treatment with an ALS inhibitor, and then developing a banana plant in the absence of further treatment with an ALS inhibitor. In certain embodiments, step (c) is performed prior to tissue culture. In certain embodiments, step (a) comprises contacting a banana embryogenic cell suspension and step (c) is performed prior to tissue culture.

In certain embodiments, the method comprises treating banana embryos with an ALS inhibitor by culturing embryos, for example starting with cells from an embryogenic cell suspension (ECS), in an embryo development medium comprising an ALS inhibitor. In certain such embodiments, the method comprises subsequently transferring embryos to an embryo development medium that does not comprise an ALS inhibitor. The inventors have found that transferring embryos to a different medium without an ALS inhibitor after selection aids the development of selected embryos. As set out above, this may be because mutation of only one ALS gene at one or two alleles may not provide complete resistance to ALS inhibitors (as might be provided with over-expression of a heterologous ALS sequence driven by a promoter), so co-edited plants might still be vulnerable to continued ALS treatment.

In certain embodiments, the invention comprises selecting at least one banana cell, such as an embryo, a banana tissue or banana plant that is resistant to treatment with an ALS inhibitor and physically separating them from cells, tissue or plants that are not resistant. In certain embodiments, the at least one selected cell, tissue or plant is transferred to a separate plate or growth medium, preferably without an ALS inhibitor.

In certain embodiments, the method comprises selecting at least one banana cell, such as an embryo, a banana tissue or banana plant that is resistant to treatment with an ALS inhibitor and subsequently selecting at least one banana cell, such as an embryo, a banana tissue or banana plant that also comprises the desired mutation in the target sequence, for example, by genotyping the cell, tissue or plant.

In certain embodiments, the method comprises treating banana embryos or a cell population with an ALS inhibitor and selection of resistant embryos or cells comprises separating developed embryos from undeveloped embryos and cell mass, and optionally culturing the developed embryos on new media. The inventors have established that physical separation of developed embryos from undeveloped embryos and cell mass improves the selection/development of target embryos. ALS treatment delays development of banana embryos, but does not necessarily kill banana embryos. Therefore, if developed embryos are not separated from undeveloped embryos and cell mass and development is allowed to continue, then it becomes harder to distinguish the resistant embryos. This is particularly important for selection in banana and using mutation of endogenous ALS genes, where ALS resistance may not be complete. Also, separating embryos may reduce embryo chimerism.

In certain embodiments, resistant embryos are transferred multiple different media, such as to a first medium and then to a second medium. In certain embodiments, the first medium comprises an ALS inhibitor and the second medium does not (or vice versa).

In certain embodiments, the ALS inhibitor is a sulfonylurea, preferably chlorsulfuron. The examples demonstrate that chlorsulfuron is effective for use with banana.

In certain embodiments, the agents comprise nucleic acid constructs and said contacting comprises Agrobacterium-mediated transformation. The examples demonstrate that Agrobacterium-mediated transformation is particularly efficient and effective for introducing mutations in endogenous ALS genes and target sequences in banana.

In certain embodiments, step (c) comprises treating the banana cell with the ALS inhibitor and said treating provides a population of banana cells and at least 90%, such as at least 95%, 96%, 97%, 98% or 99%, of the cells in the population comprise a mutation in the endogenous ALS1 gene or the endogenous ALS2 gene or both. The examples demonstrate that the methods of the invention are effective for providing populations highly enriched in cells with successful editing events.

In certain embodiments, the method does not lead to the integration of any heterologous DNA. In certain embodiments, the at least one banana embryo or plant generated by the method does not comprise heterologous DNA. The examples demonstrate that desired mutations can be efficiently introduced using the methods of the invention without the integration of any heterologous DNA, which is attractive to growers and consumers and presents greatly reduced potential issues growers wishing to obtain certain types of regulatory approval.

In preferred embodiments, the banana cell is of the autotriploid Musa acuminata ‘Cavendish’ subgroup. The examples demonstrate the effective editing of Cavendish banana cells and plants. In preferred embodiments the banana cell is from the Grand Nain cultivar.

In certain embodiments, the method comprises introducing a mutation in one or two alleles of either or both endogenous ALS genes to generate a heterozygous banana plant. The examples demonstrate that resistance to ALS inhibitors can be provided by mutating only one or two alleles of either or both endogenous ALS genes. In certain embodiments, the desired mutation(s) is/are introduced in one or two alleles of the target sequence(s).

In certain embodiments, the method comprises introducing a mutation into every allele of the endogenous ALS1 gene, or every allele of the endogenous ALS2 gene, or every allele of both the endogenous ALS1 gene and the endogenous ALS2 gene. In certain embodiments, the desired mutation(s) is/are introduced every allele of the target sequence(s). The examples demonstrate that the method of the invention is effective for generating banana plants that are homozygous for ALS resistance and a desired mutation.

In preferred embodiments, the method further comprises growing a banana plant from the at least one cell or tissue selected in step (c). In preferred embodiments, the method further comprises harvesting fruit from the banana plants and optionally processing fruit into a food product. The banana plants, banana fruits and banana fruit products of the invention advantageously carry desired mutation(s) in target sequence(s) and mutations in the endogenous ALS genes that provide resistance to ALS inhibitors. In preferred embodiments, the banana plants, banana fruits and banana fruit products do not comprise any heterologous or introduced DNA.

In a further aspect, which may be combined with any embodiment described herein, the invention provides a method of generating at least one banana embryo or plant comprising a desired sequence, the method comprising:
a. contacting a banana cell with a construct comprising the desired sequence and a sequence encoding a mutated banana acetolactate synthase gene that provides resistance to an ALS inhibitor,
b. optionally culturing the banana cell to generate a banana cell population, tissue, or plant; and
c. treating the banana cell, cell population, tissue or plant with an ALS inhibitor to select at least one banana cell, tissue or plant comprising the mutated acetolactate synthase gene and comprising the desired sequence; and
d. generating at least one banana embryo or plant from the banana cell, tissue or plant selected in step (c).

The examples demonstrate that mutated banana ALS genes are effective for providing resistance to ALS inhibitors, which makes them useful selectable markers in transgenic bananas. Preferably the desired sequence and the ALS gene are both banana sequences, to minimise the heterologous DNA introduced into the genome. In certain embodiments, at least one of the desired sequence and the ALS gene are banana sequences.

In a further aspect, which may be combined with any embodiment described herein, the invention provides method of generating at least one banana embryo or plant resistant to an ALS inhibitor, the method comprising:
a. contacting a banana cell with a construct comprising a sequence encoding a mutated banana acetolactate synthase gene that provides resistance to an ALS inhibitor or agents operable to introduce a mutation in an endogenous acetolactate synthase (ALS) gene that provides resistance to an ALS inhibitor;
b. optionally culturing the banana cell to generate a banana cell population, tissue, or plant;
e. treating the banana cell, cell population, tissue or plant with an ALS inhibitor to select at least one banana cell, tissue or plant resistant to an ALS inhibitor and comprising the mutated acetolactate synthase gene or the mutation in the endogenous acetolactate synthase gene; and
c. generating at least one banana embryo or plant from the banana cell, tissue or plant selected in step (c).

The invention also provides a banana cell, a banana embryo, or a banana plant comprising a sequence encoding a mutated banana ALS gene that provides resistance to an ALS inhibitor. The invention also provides a method of cultivating a banana plant comprising contacting the crop with an ALS inhibitor to reduce growth of other plants, wherein the banana plant comprises a sequence encoding a mutated banana acetolactate synthase gene that provides resistance to an ALS inhibitor, optionally wherein the banana plant comprises a mutation in an endogenous acetolactate synthase gene that provides resistance to an ALS inhibitor. The examples demonstrate that mutated banana ALS genes are effective for providing resistance to ALS inhibitors, which allows the production of banana plants resistant to ALS inhibitors. Either the endogenous ALS sequence is mutated or a mutated banana sequence is introduced, to minimise the heterologous DNA introduced into the genome.

In a preferred embodiment, the invention provides a method of generating at least one banana embryo or plant comprising at least one desired mutation in one or more target sequences, the method comprising:
a. contacting a banana cell with at least one nucleic acid construct encoding at least one base editor, at least one guide RNA operable to introduce the at least one desired mutation in the target sequence, and at least one guide RNA operable to introduce a mutation in an endogenous acetolactate synthase (ALS) gene that provides resistance to chlorsulfuron, wherein the mutation in the endogenous ALS gene results in the substitution Pro187Ser in ALS1 or Pro181Ser in ALS2 or both;
b. optionally culturing the banana cell to generate a banana cell population, tissue, or plant; and
c. treating the banana cell, cell population, tissue or plant with chlorsulfuron to select at least one banana cell, tissue or plant comprising the mutation in the endogenous acetolactate synthase gene and comprising the desired mutation in the target sequence; and
d. generating at least one banana embryo or plant from the banana cell, tissue or plant selected in step (c).

In a preferred embodiment, the invention provides a method of generating at least one banana embryo or plant comprising at least one desired mutation in a target sequence, the method comprising:
a. contacting a banana cell with a nucleic acid construct encoding a base editor and a guide RNA operable to introduce the at least one desired mutation in the target sequence and a nucleic acid construct encoding a base editor and a guide RNA operable to introduce a mutation in an endogenous acetolactate synthase (ALS) gene that provides resistance to chlorsulfuron, wherein the mutation in the endogenous ALS gene results in the substitution Pro187Ser in ALS1 or Pro181Ser in ALS2;
b. optionally culturing the banana cell to generate a banana cell population, tissue, or plant; and
c. treating the banana cell, cell population, tissue or plant with chlorsulfuron to select at least one banana cell, tissue or plant comprising the mutation in the endogenous acetolactate synthase gene and comprising the desired mutation in the target sequence; and
d. generating at least one banana embryo or plant from the banana cell, tissue or plant selected in step (c).

In a preferred embodiment, the invention provides a method of generating at least one banana embryo or plant comprising at least one desired mutation in a target sequence, the method comprising:
a. contacting a banana embryogenic cell suspension with one or more Agrobacterium T-DNA plasmids encoding at least one agent operable to introduce the at least one desired mutation in the target sequence and at least one agent operable to introduce a mutation in an endogenous acetolactate synthase (ALS) gene that provides resistance to an ALS inhibitor
b. culturing the banana embryogenic cell suspension;
c. culturing embryos on embryo development media comprising an ALS inhibitor to select at least one embryo comprising the mutation in the endogenous acetolactate synthase gene and comprising the desired mutation in the target sequence and
d. optionally culturing the at least one embryo to generate a plant.

In a particularly preferred embodiment, the invention provides a method of generating at least one banana embryo or plant comprising at least one desired mutation in a target sequence, the method comprising:
a. contacting a banana embryogenic cell suspension with a nucleic acid construct encoding a base editor and a guide RNA operable to introduce the at least one desired mutation in the target sequence and a nucleic acid construct encoding a base editor and a guide RNA operable to introduce a mutation in an endogenous acetolactate synthase (ALS) that provides resistance to chlorsulfuron, wherein the one or more nucleic acid constructs are present in one or more Agrobacterium T-DNA plasmids and wherein the mutation in the endogenous ALS gene results in the substitution Pro187Ser in ALS1 or Pro181Ser in ALS2;
b. culturing the banana embryogenic cell suspension;
c. culturing embryos on embryo development media comprising chlorsulfuron to select at least one embryo comprising the mutation in the endogenous acetolactate synthase gene and comprising the desired mutation in the target sequence and
d. optionally culturing the at least one embryo to generate a plant.

In a particularly preferred embodiment, the invention provides a method of generating at least one banana embryo or plant comprising at least one desired mutation in a target sequence, the method comprising:
a. contacting a banana embryogenic cell suspension with a nucleic acid construct encoding a base editor and a guide RNA operable to introduce the at least one desired mutation in the target sequence and a nucleic acid construct encoding a base editor and a guide RNA operable to introduce a mutation in an endogenous acetolactate synthase (ALS) that provides resistance to chlorsulfuron, wherein the one or more nucleic acid constructs are present in one or more Agrobacterium T-DNA plasmids and wherein the mutation in the endogenous ALS gene results in the substitution Pro187Ser in ALS1 or Pro181Ser in ALS2;
b. culturing the banana embryogenic cell suspension;
c. culturing embryos on embryo development media comprising chlorsulfuron to select at least one embryo comprising the mutation in the endogenous acetolactate synthase gene and comprising the desired mutation in the target sequence, wherein the embryo does not comprise heterologous DNA; and
d. optionally culturing the at least one embryo to generate a plant.

In a particularly preferred embodiment, the invention provides a method of generating at least one banana embryo or plant comprising at least one desired mutation in a target sequence, the method comprising:
a. contacting a banana embryogenic cell suspension with a nucleic acid construct encoding a base editor and a guide RNA operable to introduce the at least one desired mutation in the target sequence and a nucleic acid construct encoding a base editor and a guide RNA operable to introduce a mutation in an endogenous acetolactate synthase (ALS) that provides resistance to chlorsulfuron, wherein the one or more nucleic acid constructs are present in one or more Agrobacterium T-DNA plasmids and wherein the mutation in the endogenous ALS gene results in the substitution Pro187Ser in ALS1 or Pro181Ser in ALS2;
b. culturing the banana embryogenic cell suspension;
c. culturing embryos on embryo development media comprising chlorsulfuron to select a population of embryos comprising the mutation in the endogenous acetolactate synthase gene and comprising the desired mutation in the target sequence, wherein at least 1%, 2%, 3%, 4%, 5% or 10% of the embryos do not comprise a heterologous DNA; and
d. optionally culturing at least one embryo to generate a plant.

Accordingly, the invention provides the following numbered embodiments:

1. A method of generating at least one banana embryo or plant comprising at least one desired mutation in a target sequence or sequences, the method comprising:
a. contacting a banana cell with at least one agent operable to introduce the at least one desired mutation in the target sequence(s) and at least one agent operable to introduce at least one mutation in an at least one endogenous acetolactate synthase (ALS) gene so as to provide resistance to an ALS inhibitor;
b. optionally culturing the banana cell to generate a banana cell population, tissue, or plant; and
c. treating the banana cell, cell population, tissue or plant with an ALS inhibitor to select at least one banana cell, tissue or plant mutated in the at least one endogenous acetolactate synthase gene and comprising the desired mutation(s) in the target sequence(s); and
d. generating at least one banana embryo or plant from the banana cell, tissue or plant selected in step (c).
   2. The method of embodiment 1, wherein the banana cell is a protoplast or an embryogenic cell, such as wherein the banana cell is a protoplast in a protoplast cell suspension or an embryogenic cell in an embryogenic cell suspension and step (a) comprises contacting the protoplast cell suspension or embryogenic cell suspension.
   3. The method of any preceding embodiment, wherein the agents operable to introduce the mutations comprise a base editor and guide RNAs or comprise at least one nucleic acid construct that encodes a base editor and guide RNAs.
   4. The method of embodiment 3, wherein the base editor is a fusion polypeptide comprising a modified Cas, such as nCas9 or dCas9, and a cytidine deaminase moiety or an adenine deaminase moiety, such as a base editor selected from the group consisting of APOBEC, BE1, BE2, BE3, HF-BE3, BE4, BE4max, BE4-GAM, YE1-BE3, EE-BE3, YE-BE3, YEE-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, ABE7.10*, xABE, ABESa, VQR-ABE, VRER-ABE, and SaKKH-ABE.
   5. The method of embodiment 1 or embodiment 2, wherein the agents operable to introduce the mutations comprise a sequence-specific endonuclease, optionally guide RNAs and optionally donor templates, or comprise at least one nucleic acid construct that encodes a sequence-specific endonuclease, optionally guide RNAs and optionally donor templates.
   6. The method of embodiment 5, wherein the sequence-specific endonuclease is a meganuclease, a zinc finger nuclease (ZFN), a transcription-activator like effector nuclease (TALEN), a homing endonuclease or a CRISPR-associated endonuclease, such as a modified CRISPR-associated endonuclease.
   7. The method of any preceding embodiment, wherein the agents operable to introduce the at least one desired mutation in the target sequence comprise a guide RNA targeting the target sequence and the agents operable to introduce the mutation in the endogenous ALS gene comprise a guide RNA targeting the endogenous ALS1 or endogenous ALS2 gene or both, or wherein the agents operable to introduce the at least one desired mutation in the target sequence comprise at least one nucleic acid construct encoding a guide RNA targeting the target sequence and the agents operable to introduce the mutation in the endogenous ALS gene comprise at least one nucleic acid construct encoding a guide RNA targeting the endogenous ALS1 or endogenous ALS2 gene or both.
   8. The method of any preceding embodiment, wherein the agents operable to introduce the mutations are constructs present on one or more, such as two, plasmids.
   9. The method of any preceding embodiment, wherein the agents comprise nucleic acid constructs and said contacting comprises Agrobacterium-mediated transformation, particle bombardment, protoplast transformation, nanoparticle-mediated transfection or electroporation.
   10. The method of any of any preceding embodiment, wherein the agents operable to introduce the mutations are delivered to the banana cell as ribonucleoprotein complexes.
   11. The method of any preceding embodiment, wherein step (c) comprises incubating an embryogenic cell suspension in the presence of an ALS inhibitor or step (c) comprises culturing embryos on embryo development medium (EDM) comprising an ALS inhibitor.
   12. The method of embodiment 11, wherein step (c) comprises culturing embryos on embryo development media comprising an ALS inhibitor embryogenic cell suspension for 8-20 weeks, such as 8-16, 10-16, 10-14, 12-14 or around 13 weeks.
   13. The method of embodiment 11 or 12, wherein step (c) comprises culturing embryos on embryo development media comprising an ALS inhibitor embryogenic cell suspension at a concentration of 10-80 μg/L, such as 10-70, 15-65, 15-55, 20-55, 20-50, 20-40, 20-30 or around 25 μg/L.
   14. The method of embodiment 11, 12 or 13, wherein step (c) comprises incubating the embryogenic cell suspension in the presence of an ALS inhibitor for at least 3 days, such as at least 5, 10 or 15 days, optionally for less than 30 days, such as less than 25, 20, 15 or 10 days.
   15. The method of any of embodiments 11-14, wherein step (c) comprises incubating the embryogenic cell suspension in the presence of an ALS inhibitor at a concentration of at least 500 μg/L, such as at least 1, 2, 3, 4, or 5 mg/L.
   16. The method of any preceding embodiment, comprising selecting at least one embryo following treatment with an ALS inhibitor and developing a banana plant in the absence of further treatment with an ALS inhibitor.
   17. The method of any preceding embodiment, additionally comprising subsequently transferring embryos to an embryo development medium that does not comprise an ALS inhibitor.
   18. The method of any preceding embodiment, comprising selecting at least one banana cell, such as an embryo, a banana tissue or banana plant that is resistant to treatment with an ALS inhibitor and physically separating them from cells, tissue or plants that are not resistant and optionally transferring the at least one selected cell, tissue or plant to a separate plate or growth medium, preferably without an ALS inhibitor.
   19. The method of any preceding embodiment comprising selecting at least one banana cell, such as an embryo, a banana tissue or banana plant that is resistant to treatment with an ALS inhibitor and subsequently selecting at least one banana cell, such as an embryo, a banana tissue or banana plant that also comprises the desired mutation in the target sequence, for example, by genotyping the cell, tissue or plant.
   20. The method of any preceding embodiment, wherein the agents operable to introduce the at least one desired mutation in the target sequence(s) and/or the agents operable to introduce the mutation in the endogenous ALS gene(s) comprise a nucleic acid construct that additionally encodes a selectable marker, and wherein the method optionally comprises selecting an embryo or plant that does not carry the selectable marker.
   21. The method of any preceding embodiment, wherein the endogenous acetolactate synthase gene is the endogenous acetolactate synthase 1 (ALS1) gene or the endogenous acetolactate synthase 2 (ALS2) gene or wherein mutations are introduced in both ALS1 and ALS2.
   22. The method of any preceding embodiment, wherein the mutation in the endogenous ALS gene introduces a substitution in the encoded amino acid sequence, preferably at Pro-187 in banana ALS1 or Pro-181 in ALS2, most preferably Pro187Ser in ALS1 or Pro181Ser in ALS2.
   23. The method of any preceding embodiment, wherein step (a) comprises contacting the banana cell with one or more agents operable to mutate both the endogenous ALS1 and ALS2 genes so as to provide resistance to an ALS inhibitor.
   24. The method of any preceding embodiment, wherein the ALS inhibitor is a sulfonylurea, imidazolinone, triazolopyrimidine, pyrimidinyl oxybenzoate, or sulfonylamino carbonyl triazolinones, preferably wherein the ALS inhibitor is chlorsulfuron.
   25. The method of any preceding embodiment, wherein step (a) comprises contacting a banana embryogenic cell suspension and wherein step (c) is performed prior to tissue culture.
   26. The method of any preceding embodiment, wherein step (c) provides a population of banana cells and at least 90%, such as at least 95%, 96%, 97%, 98% or 99%, of the cells in the population comprise mutated ALS, optionally wherein at least 5%, 10% or 15% of the cells do not comprise heterologous DNA, further optionally wherein at least 1%, 2%, 3%, 4% or 5% of the cells comprise the at least one mutation in the endogenous acetolactate synthase gene and comprise the desired mutation in the target sequence.
   27. The method of any preceding embodiment, wherein the at least one banana cell, tissue, embryo or plant generated by the method does not comprise heterologous DNA.
   28. The method of any preceding embodiment, wherein the target sequence is one or more of ACO. ACS or PPO, such as ACO1, ACO2, ACS1, ACS2 or PPO2.
   29. The method of any preceding embodiment, further comprising growing a banana plant from at least one cell or tissue selected in step (c).
   30. The method of any preceding embodiment, further comprising harvesting a fruit from said banana plant.
   31. The method of embodiment 30, further comprising processing said fruit into a food product.
   32. The method of any of the preceding embodiments, wherein the banana cell is a variety/cultivar of the autotriploid Musa acuminata ‘Cavendish’subgroup, such as Grand Nain.
   33. A banana cell, embryo, cell population, tissue or plant generated by the method according to any preceding embodiment.
   34. A banana cell, a banana embryo or a banana plant comprising a mutation in the endogenous ALS1 gene or the endogenous ALS2 gene, or both, that provides resistance to an ALS inhibitor.
   35. A population of banana embryos, wherein at least 90%, such as at least 95%, 96%, 97%, 98% or 99%, of the embryos comprise a mutated endogenous ALS1 gene or mutated endogenous ALS2 gene, or both, that provides resistance to an ALS inhibitor, optionally wherein at least part of the embryos comprising a mutation in ALS1 and/or ALS2 that provides resistance to an ALS inhibitor do not comprise heterologous DNA.
   36. The banana cell or plant of embodiment 34 or the population of embodiment 35, additionally comprising at least one mutation in at least one target sequence that provides pest resistance or a phenotypically desirable trait, optionally wherein said target sequence is ACO, ACS or PPO, such as ACO1, ACO2, ACS1, ACS2 or PPO2, further optionally wherein the mutation is introduction of a stop codon.
   37. The banana cell, a banana embryo or plant of embodiment 34 or the population of embodiment 35, additionally comprising a mutation in a silencing sequence alters its silencing specificity such that when expressed it reduces the expression of a target gene, optionally wherein said target gene is a pest gene or wherein said reduced expression of the target gene provides a phenotypically desirable trait.
   38. A method of generating at least one banana embryo or plant comprising a desired sequence or sequences, the method comprising:
a. contacting a banana cell with at least one construct carrying the desired sequence(s) and a sequence encoding at least one mutated banana acetolactate synthase gene that provides resistance to an ALS inhibitor;
b. optionally culturing the banana cell to generate a banana cell population, tissue, or plant;
c. treating the banana cell, cell population, tissue or plant with an ALS inhibitor to select at least one banana cell, tissue or plant comprising the at least one mutated acetolactate synthase gene and comprising the desired sequence(s); and
d. generating at least one banana embryo or plant from the banana cell, tissue or plant selected in step (c).
   39. A method of generating at least one banana embryo or plant resistant to an ALS inhibitor, the method comprising:
a. contacting a banana cell with at least one construct carrying sequence encoding at least one mutated banana acetolactate synthase gene that provides resistance to an ALS inhibitor or at least one agent operable to introduce a mutation in an endogenous acetolactate synthase (ALS) gene that provides resistance to an ALS inhibitor;
b. optionally culturing the banana cell to generate a banana cell population, tissue, or plant;
c. treating the banana cell, cell population, tissue or plant with an ALS inhibitor to select at least one banana cell, tissue or plant resistant to an ALS inhibitor and comprising the mutated acetolactate synthase gene or the mutation in the endogenous acetolactate synthase gene; and
d. generating at least one banana embryo or plant from the banana cell, tissue or plant selected in step (c).
   40. The method of embodiment 38 or 39, further comprising growing a banana plant from the at least one cell or tissue selected in step (c).
   41. The method of any of embodiments 38-40, further comprising harvesting a fruit from said banana plant.
   42. The method of embodiment 41, further comprising processing said fruit into a food product.
   43. A banana cell, embryo, tissue or plant generated by the method according to of embodiments 38-40.
   44. A banana cell, a banana embryo or a banana plant comprising a sequence encoding a mutated banana acetolactate synthase gene that provides resistance to an ALS inhibitor mutation.
   45. A population of banana embryos, wherein at least 90%, such as at least 95%, 96%, 97%, 98% or 99%, of the embryos comprise a sequence encoding a mutated acetolactate synthase gene that provides resistance to an ALS inhibitor.
   46. The banana cell, embryo or plant of embodiment 44 or the population of embodiment 45, additionally comprising a desired sequence that provides pest resistance or a phenotypically desirable trait.
   47. The banana cell, embryo or plant of embodiment 44 or the population of embodiment 45, additionally comprising a desired sequence that when expressed reduces the expression of a target gene, optionally wherein said target gene is a pest gene or wherein said reduced expression of the target gene provides a phenotypically desirable trait.
   48. A method of cultivating a banana plant comprising contacting the crop with an ALS inhibitor to reduce growth of other plants, wherein the banana plant comprises a sequence encoding a mutated banana acetolactate synthase gene that provides resistance to an ALS inhibitor, or wherein the banana plant comprises a mutation in an endogenous acetolactate synthase gene that provides resistance to an ALS inhibitor, and wherein said other plants are susceptible to said ALS inhibitor, optionally wherein said ALS inhibitor is chlorsulfuron.
   49. Use of an ALS inhibitor as a herbicide for cultivating a banana plant grown according to any preceding embodiment.

### BRIEF DESCRIPTION OF THE FIGURES

FIG. 1—Qualitative analysis of number of embryos after chlorsulfuron (CSF) treatment of banana embryogenic cell suspensions. The ECS samples were exposed to the toxin for a short period of time (3, 5, 10, 15 days exposure) and then they were inoculated into embryo development medium (EDM; without CSF). Number of embryos was assessed 2 months post exposure.

FIG. 2—Banana ECS after 5, 10 and 15 days of CSF treatment (0, 0.5, 5, 50 and 100 mg/L).

FIG. 3—Banana embryos developed for 3 months post CSF treatment for 5, 10 and 15 days.

FIG. 4—Development of banana plantlets in RM (rooting medium) supplemented with CSF (0, 0.05, 0.5 and 5 mg/L) for 6 weeks.

FIG. 5—Development of banana plantlets in RM medium supplemented with CSF (0, 0.05, 0.5, 5, 50 and 100 mg/L) for four weeks.

FIG. 6—Development of banana plantlets in RM medium a month after CSF spraying (0, 0.05, 0.5, 5, 50 and 100 mg/L). CSF solutions were prepared in sterile deionised water (spray volume 1 mL per phytotray (five shoots in a tray), one spray at day 0).

FIG. 7—Development of banana plantlets in RM medium a month after CSF spraying (0, 0.05, 0.5, 5, 50 and 100 mg/L). CSF solutions were prepared in 0.015% Silwett L-77 (spray pattern as discussed for FIG. 6).

FIG. 8—Development of banana plantlets in RM medium a month after CSF weekly spraying (0, 0.05, 0.5, 5, 50 and 100 mg/L). CSF solutions were prepared in 0.015% Silwett L-77 (spray volume 1 mL per phytotray (five shoots in a tray), sprays at day 0 and after week one, two and three).

FIG. 9—Developing of banana plantlets in RM medium a month after CSF spraying (0, 0.05, 0.5, 5, 50 and 100 mg/L). The RM medium also contained 0.05 mg/L of CSF as an adjuvant. CSF solutions were prepared in 0.015% Silwett L-77 (spray pattern as discussed for FIGS. 6 and 7).

FIG. 10—Developing of banana plantlets in RM medium a month after CSF weekly spraying (0, 0.05, 0.5, 5, 50 and 100 mg/L). The RM medium also contained 0.05 mg/L of CSF as an adjuvant. CSF solutions were prepared in 0.015% Silwett L-77 (spray pattern as discussed for FIG. 8).

FIG. 11—RPKM data for banana ALS1 and ALS2 showing the relative, normalised expression profile of these genes across different banana tissues (Reads Per Kilobase of transcript, per Million mapped reads—RPKM—is a normalised unit of transcript expression).

FIG. 12—Expression profile of ALS1 and ALS2 in different developmental stages of banana, measured using RT-qPCR. Data shown are normalised, and relative to ALS1 in ECS. Error bars are standard error of the mean. “ddct” refers to the delta delta CT method and is a standard method for measuring relative normalised expression.

FIG. 13—Plasmid map for pMOL_0632_EI

FIG. 14A—Plasmid map for pMOL_0630_EI

FIG. 14B—Plasmid map for pMOL_0628_EI

FIG. 15—A. Plasmid construct cartoon of the construct used to generate precise edits in banana ALS1 or ALS2 gene. B. The specific ALS1 amino acid change introduced into banana to confer resistance to chlorsulfuron, P187S. Also indicated is a tolerated silent mutation C to T in the valine codon preceding P187. C. Specific sequence sections of ALS1 and ALS2 in banana, showing amino acid residues and corresponding nucleotide triplets (SEQ ID NOs:57-59). Underlined are the specific chlorsulfuron resistance targets for ALS1 and ALS2; P187 and P181, respectively. Highlighted in grey is the position of the sgRNA sequence plus CGG protospacer adjacent motif (PAM).

FIG. 16—Agrobacterium-transformed banana embryos developed in the presence of CSF 25 and 50 μg/L for three months.

FIG. 17—Expression of mcherry fluorescence in transformed ALS1 (pMol_0630) banana embryos growing in CSF (0, 25 and 50 μg/L) for 3 months. Mcherry expression indicates the integration of the T-DNA. Pictures were taken using bright field (BF) and an mcherry fiker. Possible transiently edited embryos are highlighted in the green boxes. 20× magnification; Exposure BF—10 ms and mcherry—200 ms.

FIG. 18—Expression of mcherry fluorescence in transformed ALS2 (pMol_0632) banana embryos growing in CSF (0, 25 and 50 μg/L) for 3 months. Mcherry expression indicates the integration of the T-DNA. Pictures were taken using bright field (BF) and mcherry filter. Possible transiently edited embryos are highlighted in the green boxes. 20× magnification; Exposure BF—10 ms and mcherry—200 ms.

FIG. 19—Table summarising editing frequency and transgenic embryo analysis following CSF selection.

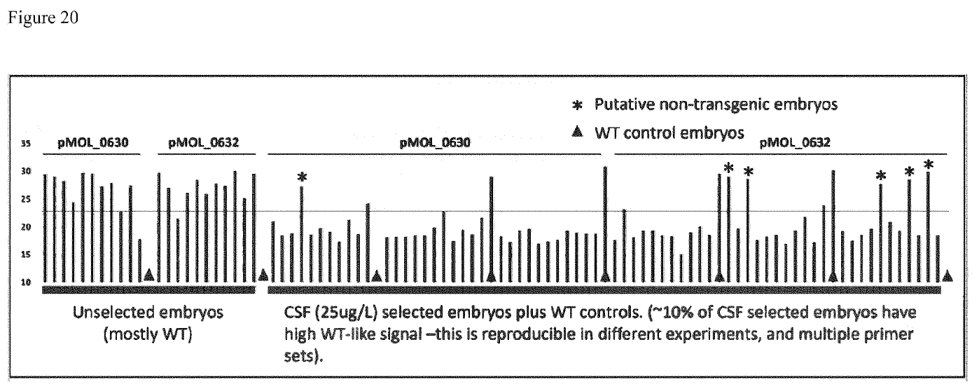

FIG. 20—Normalised Ct values of amplicons from T-DNA indicate the presence of transgenes in immature banana embryos. We set a threshold (>25 cycles) based on WT (wild type) controls for each individual primer set. Data are normalised using a housekeeping gene to account for minor DNA concentration differences. Between 6-15% of these embryos appear to be non-transgenic by qPCR analysis.

FIG. 21—ALS1 sequencing results

FIG. 22—ALS2 sequencing results

FIG. 23—PBE #1 and PBE #2 embryo genotyping data (ALS only, no trait)

FIG. 24—A selection of the different plant genotypes identified in PBE #3.

FIG. 25—Comparison of agrobacterium transformation and particle bombardment.

### DETAILED DESCRIPTION OF THE INVENTION

Treatment with an ALS Inhibitor

The methods of the invention utilise treatment of a banana cell, cell population, tissue or plant with an ALS inhibitor to select for at least one banana cell, tissue or plant comprising a mutation in an endogenous acetolactate synthase gene and comprising a desired mutation in a target sequence. Either or both of ALS1 and ALS2 may be mutated and, similarly, one or more target sequences may each comprise one or more mutations. For example, there could be one target mutation, multiple mutations in one target sequence or multiple mutations across different target sequences with the same or different numbers of mutations in different target sequences.

In certain embodiments, the ALS inhibitor for use in the methods of the invention is an imidazolinone, a pyrimidinylthiobenzoate, a sulfonylaminocarbonyluiazolinone, a sulfonylurea, or a triazolopyrimidine. Preferably the ALS inhibitor is a sulfonylurea. Preferred imidazolinones include imazamethabenz-methyl, imazamox, imazapic, imazapyr, imazaquin and imazethapyr. Preferred pyrimidinylthiobenzoates include bispyribac-sodium and pyrithiobac-sodium. Preferred sulfonylaminocarbonyltriazolinones include flucarbazone-sodium and propoxycarbazone-sodium. Preferred sulfonylureas include bensulfuron-methyl, chlorimuron-ethyl, chlorsulfuron, foramsulfuron, halosulfuron-methyl, mesosulfuron-methyl, metsulfuron-methyl, nicosulfuron, primisulfuron-methyl, prosulfuron, rimsulfuron, sulfometuron-methyl, sulfosulfuron, thifensulfuron-methyl, triasulfuron, tribenuron-methyl, trifloxysulfuron-sodium and triflusulfuron-methyl. Preferred triazolopyrimidines include cloransulam-methyl, diclosulam, florasulam, flumetsulam, penoxsulam and pyroxsulam. Most preferably, the ALS inhibitor for use in the methods of the invention is chlorsulfuron. The ALS mutation(s) introduced may provide resistance to any one or more ALS inhibitors, for example a single mutation could provide resistance against a plurality of inhibitors and multiple mutations could provide resistance against different inhibitors.

In preferred embodiments, the method comprises treatment of an embryo, or a population of embryos, such as culturing embryos on embryo development media comprising an ALS inhibitor. “Culturing embryos”, “incubating embryos” and “treating embryos” as used herein encompasses generating embryos from embryogenic cells and incubating developing embryos. In such embodiments, the embryos are preferably incubated on embryo development medium comprising an ALS inhibitor. This means that embryogenic cells are cultured on embryo development medium as they develop into embryos. In certain embodiments, the embryos are cultured until fully developed embryos are observed. In certain embodiments, the embryos are incubated on embryo development medium comprising an ALS inhibitor for 8-20 weeks, such as 8-16, 10-16, 10-14, 12-14 or around 13 weeks. In certain embodiments, the embryos are incubated on embryo development medium comprising an ALS inhibitor at a concentration of 10-80 μg/L, such as 10-70, 15-65, 15-55, 20-55, 20-50, 20-40, 20-30 or around 25 μg/L. In certain embodiments, the embryos are incubated on embryo development medium comprising an ALS inhibitor at a concentration of 10-80 μg/L, such as 20-70, 25-65, 30-60, 40-60, 40-55, 45-55 or around 50 μg/L. In certain embodiments the embryos are incubated on embryo development medium comprising an ALS inhibitor at a concentration of 10-80 μg/L, such as 10-70, 15-65, 15-55, 20-55, 20-50, 20-40, 20-30 or around 25 μg/L or a concentration of 10-80 μg/L, such as 20-70, 25-65, 30-60, 40-60, 40-55, 45-55 or around 55 μg/L, for 8-20 weeks, such as 8-16, 10-16, 10-14, 12-14 or around 13 weeks.

In certain embodiments, the embryos or embryogenic cell suspension are allowed to recover following transformation and before ALS inhibitor selection, such as for 1-14, 3-10, 5-10 or around 7 days.

In certain embodiments, the method comprises treatment of an embryogenic cell suspension. In certain embodiments, the embryogenic cell suspension is incubated in the presence of an ALS inhibitor for at least 3 days, such as at least 5, 10 or 15 days, optionally for less than 30 days, such as less than 25, 20, 15 or 10 days. In certain embodiments, the embryogenic cell suspension is incubated in the presence of an ALS inhibitor at a concentration of at least 500 μg/L, such as at least 1, 2, 3, 4, or 5 mg/L. In certain embodiments, the embryogenic cell suspension is incubated in the presence of an ALS inhibitor at a concentration of at least 500 μg/L, such as at least 1, 2, 3, 4, or 5 mg/L for at least 3 days, such as at least 5, 10 or 15 days, optionally for less than 30 days, such as less than 25, 20, 15 or 10 days.

In certain embodiments, the method comprises treating banana embryos with an ALS inhibitor by culturing embryos, for example starting with cells from an ECS, in an embryo development medium comprising an ALS inhibitor. In certain such embodiments, the method comprises subsequently transferring embryos to an embryo development medium that does not comprise an ALS inhibitor. In certain embodiments, the method comprises culturing embryos from a cell suspension, cell population or cell mass in an embryo development medium comprising an ALS inhibitor, observing the development of embryos that are resistant to the ALS inhibitor, and physically separating those embryos from the remaining cells that are less developed or not developed. In certain embodiments, the developed embryos are transferred to an embryo development medium that does not comprise an ALS inhibitor.

In certain embodiments, the invention comprises selecting at least one banana cell, such as an embryo, a banana tissue or banana plant that is resistant to treatment with an ALS inhibitor and physically separating them from cells, tissue or plants that are not resistant. In certain embodiments, the at least one selected cell, tissue or plant is transferred to a separate plate or growth medium, preferably without an ALS inhibitor.

In certain embodiments, treatment of cells with an ALS inhibitor, such as culturing embryos in the presence of an ALS inhibitor, provides a population of cells or embryos, of which at least 90% are resistant to the ALS inhibitor, such as at least 95%, 96%, 97%, 98% or 99%. In some of said cells or embryos the nucleic acid constructs used to introduce the mutation may be integrated, in which case the cells or embryos will be considered transgenic. However, in certain embodiments, at least 5%, at least 10% or at least 15% of the cells or embryos will have been edited at an endogenous ALS gene (i.e. either ALS1 or ALS2 or both) and will not have any integrated heterologous DNA and so will be considered non-transgenic. Furthermore, some of the cells or embryos will also be edited at a target sequence and will comprise a desired mutation. In certain embodiments, at least 1%, 2%, 3%, 4% or 5% of cells or embryos comprise the mutation in the endogenous acetolactate synthase gene and comprise a desired mutation in a target sequence.

In certain embodiments, the method comprises treatment of a plant or plantlet. In certain embodiments, the method comprises spraying a plant or plantlet with an ALS inhibitor. In certain embodiments, spray additionally comprises an adjuvant, such as Silwett L-77. In certain embodiments, the plants or plantlets are sprayed with an ALS inhibitor at a concentration of at least 1 mg/L, such as at least 2, 3, 4 or 5 mg/L or 1-10 mg/L or 2-7 mg/L. In certain embodiments, the plants or plantlets are sprayed approximately once a week. In certain embodiments, the plants or plantlets are sprayed weekly for four weeks. In certain embodiments, the first weekly spraying is at the start of the first week. In certain embodiments, the plants or plantlets are sprayed four times, preferably once a week. In certain embodiments, the method comprises growing plants or plantlets in rooting medium to which an ALS inhibitor has been added. In certain embodiments, the rooting medium comprises an ALS inhibitor at less than 1 mg/L, such as less than 0.5 mg/L or less than 0.1 mg/L, such as 0.01-0.09 or 0.002-0.06 mg/L or about 0.005 mg/L. In certain embodiments the plantlets are grown in ALS inhibitor-containing rooting medium for 2-6 weeks, such as 3-5 weeks, around one month, or 4 weeks. In certain embodiments, the treatment comprises spraying the plants or plantlets and growing them in rooting medium comprising an ALS inhibitor. In certain embodiments, the plants or plantlets are exposed to an ALS inhibitor for 2-6 weeks, such as 3-5 weeks, around one month, or 4 weeks.

In certain embodiments, the method comprises culturing embryos in the presence of an ALS inhibitor, preferably using the concentrations and/or time periods set out above, and does not comprise any other treatment with an ALS inhibitor. In such embodiments the method does not comprise treating plants or plantlets with an ALS inhibitor and does not comprise incubating an ECS with an ALS inhibitor. In such embodiments, the ECS following transformation is incubated in a medium that does not comprise an ALS inhibitor and plants and plantlets are grown in a medium that does not comprise an ALS inhibitor.

### Agents Operable to Introduce Mutations

The methods of the invention utilise agents to introduce at least one desired mutation in a target sequence and agents to introduce a mutation in an endogenous ALS gene. Generally different agents will be used to target the target sequence(s) and the ALS gene(s), in particular, different sequence-specific agents, but the agents may optionally share components, such as endonucleases or base editors. Accordingly, the method may optionally comprise contacting the banana cell with one base editor or endonuclease, or one construct encoding a base editor or endonuclease, or one or more constructs encoding the same base editor or endonuclease, which is operable to introduce both a mutation in the target sequence and a mutation in an ALS gene. In such embodiments, the agents will also comprise sequence-specific agents. The base editor or endonuclease could be encoded by the construct that encodes the sequence-specific agent or agents targeted to the ALS gene, or the construct that encodes the sequence-specific agent or agents targeted to the target sequence, or a single construct with multiple sequence-specific components could be used. Preferably, both the agents used to introduce at least one desired mutation in a target sequence and the agents used to introduce a mutation in an endogenous ALS gene will comprise the same or similar agents, such as both will comprise the same or similar base editor (for example either because a single base editor or construct encoding the base editor is used or because one or more constructs encoding multiple copies of the same base editor are used), because if both mutations are introduced in the same manner this may increase the likelihood that banana cells or plants resistant to ALS inhibition carry the desired mutation in the target sequence. It is also possible that different base editors/endonucleases could be used, for example the ALS and target gene editing agents could comprise different base editors, different endonucleases or a combination thereof.

In preferred embodiments, the agents collectively comprise a base editor and guide RNAs or comprise at least one nucleic acid construct that encodes a base editor and guide RNAs. Base editors are effective for introducing specific mutations without requiring the integration of heterologous DNA, as shown in the examples. The agents may comprise one guide RNA or multiple guide RNAs targeted to the endogenous ALS gene and/or one guide RNA or multiple guide RNAs targeted to the target sequence. Optionally the agents comprise one guide RNA targeted to the endogenous ALS gene and multiple guide RNAs targeted to the target sequence.

Base editing is a genome editing approach that uses components from CRISPR systems together with other enzymes to directly install point mutations into cellular DNA or RNA without making double-stranded DNA breaks. In particular, modified Cas, such as dCas9 or nCas9, can be used together with other enzymes (generally as a fusion protein) for base-editing. DNA base editors comprise a catalytically disabled nuclease fused to a nucleobase deaminase enzyme and, in some cases, a DNA glycosylase inhibitor. RNA base editors achieve analogous changes using components that target RNA. Base editors directly convert one base or base pair into another, enabling the efficient installation of point mutations in non-dividing cells without generating excess undesired editing by-products (Rees and Liu (2018), “Base Editing: Precision Chemistry on the Genome and Transcriptome of Living Cells”, Nature Reviews Genetics, 19(12): 770-788). According to some embodiments, the base editor comprises nCas9 or dCas9 fused to an adenine or cytidine deaminase, and optionally fused to a DNA glycosylase inhibitor. A preferred base editor is a fusion comprising nCas9(D10A), cytidine deaminase APOBEC and uracil glycosylase inhibitor. Exemplary suitable base editors are provided in Zong et al. Nat Biotechnol. 2017, 35(5):438-440). In certain embodiments, the base editor has both adenine and cytidine deaminase activity and is a dual-deaminase base editor (Grünewald, et al., 2020, Nature Biotechnology, 38: 861-864).

In preferred embodiments, the base editor is encoded by SEQ ID NO:17 or a sequence having at least 70, 80, 85, 90, 95, 98 or 99% sequence identity to SEQ ID NO:17. In preferred embodiments, the base editor comprises SEQ ID NO:18 or a sequence having at least 70, 80, 85, 90, 95, 98 or 99% sequence identity to SEQ ID NO:18.

Further base editors contemplated include APOBEC, BE1, BE2, BE3, HF-BE3, BE4, BE4max, BE4-GAM, YE1-BE3, EE-BE3, YE-BE3, YEE-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, ABE7.10*, xABE, ABESa, VQR-ABE, VRER-ABE, and SaKKH-ABE (Rees and Liu (2018), “Base Editing: Precision Chemistry on the Genome and Transcriptome of Living Cells”, Nature Reviews Genetics, 19(12): 770-788, and references therein). Further contemplated base editors include modified Cas12 or Cas13, such as dCas12, dCas13, dCas12a, dCas12b, dCas13a, dCas13b dCas13c, or dCas13d.

Base editors are generally used in conjunction with a guide RNA to introduce specific mutations in a target sequence. A guide RNA (or gRNA) used in the present invention is specific to the target sequence or the ALS gene. In some embodiments of the invention, the one or more guide RNAs comprises a sequence selected from the group consisting of: SEQ ID NO:19 and SEQ ID NO:20, or a sequence having at least 90, 95, 98 or 99% sequence identity to SEQ ID NO:19 and SEQ ID NO:20. In certain embodiments of the invention, the guide RNAs comprise a sequence selected from the group consisting of: SEQ ID NO:19 and SEQ ID NO:20, or a sequence having at least 90, 95, 98 or 99% sequence identity to SEQ ID NO:19 and SEQ ID NO:20 (for targeting ALS1 and/or ALS2) and comprise a sequence targeting a target gene, such as ACO, ACS or PPO (in particular, ACO1, ACO2, ACS1, ACS2 or PPO2), preferably wherein the sequence targeting a target gene is selected from the group consisting of: SEQ ID NO:21-26, or a sequence having at least 90, 95, 98 or 99% sequence identity to SEQ ID NO:21-26.

In further embodiments, the agents comprise a sequence-specific endonuclease, guide RNAs and optionally donor templates, or comprise at least one nucleic acid construct that encodes a sequence-specific endonuclease, guide RNAs and optionally donor templates.

Preferably the endonuclease mediates the introduction of specific mutations into the ALS gene and the target sequence, such as via HDR (homology directed repair), rather than a random indel, such as by NHEJ.

Accordingly, in certain embodiments, the agents comprise at least one endonuclease, at least one donor template that comprises at least one mutation in at least one endogenous ALS gene, and at least one donor template that comprises at least one desired mutation in the target sequence. Similarly, in certain embodiments, the agents comprise at least one nucleic acid construct encoding at least one endonuclease, at least one donor template that comprises at least one mutation in at least one endogenous ALS gene, and at least one donor template that comprises at least one desired mutation in the target sequence. Preferably the donor templates are donor oligonucleotides. Such embodiments are effective for introducing the mutations without requiring integration of any heterologous DNA sequence, and accordingly in certain embodiments, the method does not lead to the integration of any heterologous DNA, and in certain embodiments, the at least one banana embryo or plant generated by the method does not comprise heterologous DNA. As with other embodiments of the invention, selecting for plants that have undergone HDR editing at the endogenous ALS locus with an ALS inhibitor will enrich for plants that are also HDR-edited (co-HDR) at the target sequence.

As used herein, the term “donor oligonucleotide” or “donor template” refers to exogenous nucleotides, i.e. externally introduced into the banana cell to generate a precise change in the genome. The donor oligonucleotides may be synthetic. In certain embodiments, the donor oligonucleotides are RNA oligonucleotides. In certain embodiments, the donor oligonucleotides are DNA oligonucleotides. In certain embodiments, the donor oligonucleotides comprise single-stranded donor oligonucleotides (ssODN), double-stranded donor oligonucleotides (dsODN), double-stranded DNA (dsDNA), double-stranded DNA-RNA duplex (DNA-RNA duplex) double-stranded DNA-RNA hybrid, single-stranded DNA-RNA hybrid, single-stranded DNA (ssDNA), double-stranded RNA (dsRNA), single-stranded RNA (ssRNA). In certain embodiments, the donor oligonucleotides are provided in a non-expressed vector format or oligo. In certain embodiments, the donor oligonucleotides comprise a DNA donor plasmid (e.g. circular or linearized plasmid).

In preferred embodiments, the donor templates introduce only minimal changes to the endogenous ALS sequence and the target sequence. In certain embodiments, the donor templates comprise 1-40, such as 5-40, 5-30, 5-20, 3-40, 3-30, 3-20, 5-15, 3-10, 10-30, or 10-20 nucleotide additions, deletions and substitutions relative to the sequence encoding the endogenous ALS sequence and the target sequence. In preferred embodiments, the donor template that comprises the at least one mutation in at least one endogenous ALS gene comprises a single nucleotide substitution, as set out in more detail in the section below titled “Mutation in an endogenous acetolactate synthase gene”.

Any appropriate design for HDR donor templates may be used. In certain embodiments, the donor templates comprise homology arms, such as two homology arms each of 10-1000 nucleotides in length, such as 100-1000, 200-1000, 200-800, 300-800, 300-700, 400-600 nucleotides. In certain embodiments, the templates comprise chemical modifications, such as phosphorothioate modifications. Modifications could prevent ligation of linear donor and/or enhance its stability. According to one embodiment, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides. According to a specific embodiment, the donor oligonucleotides comprising the ssODN (e.g. ssDNA or ssRNA) comprise about 200-500 nucleotides. According to further embodiments the donor oligonucleotides comprising the ssODN (e.g. ssDNA or ssRNA) comprise about 300-500 nucleotides or about 400-500 nucleotides. According to a specific embodiment, the donor oligonucleotides comprising the dsODN (e.g. dsDNA or dsRNA) comprise about 250-5000 nucleotides.

In certain embodiments, the donor templates are 40-200 nucleotides in length, such as 50-180, 60-150, 60-120, 70-120, 70-110, 80-110 or 80-100 nucleotides.

Endonucleases are enzymes that cleave the phosphodiester bond within a polynucleotide chain, and include restriction endonucleases that cleave DNA at specific sites without damaging the bases. Restriction endonucleases include Type 1, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site. Endonucleases allow for precision genetic engineering of eukaryotic genomes, such as plant genomes. In some embodiments, the endonuclease is inactivated and catalytically dead, such as in dCas9, as discussed further below.

In some embodiments, the endonuclease is selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease. According to some embodiments, the endonuclease is a CRISPR-associated endonuclease, optionally wherein the CRISPR-associated endonuclease is Cas9. Each possibility represents a separate embodiment of the present invention.

As used herein, a “CRISPR-associated endonuclease” (or “Cas”) refers to an endonuclease having an RNA-guided polynucleotide-editing activity and is one of the components of the CRISPR/Cas system for genome editing, which uses at least one additional component, a “guide RNA” (gRNA). In some embodiments of the invention, the “CRISPR-associated endonuclease” is a “Cas9 endonuclease” (or “Cas9”). According to some embodiments, the “CRISPR-associated endonuclease” may be any Cas9 known in the art, such as, but not limited to. SpCas9, SaCas9, FnCas9, NmCas9, StlCas9, BlatCas9 (Shota Nakade, Takashi Yamamoto & Tetsushi Sakuma (2017), Bioengineered, 8:3, 265-273, and references therein). In other embodiments, the “CRISPR-associated endonuclease” may be Cpf1, such as, but not limited to, AsCpf1 or LbCpf1 (Shota Nakade, Takashi Yamamoto & Tetsushi Sakuma (2017), Bioengineered, 8:3, 265-273, and references therein). In further embodiments, the “CRISPR-associated endonuclease” may be any known Cas12 or Cas13, such as dCas12, dCas13, dCas12a, dCas12b, dCas13a, dCas13b dCas13c, or dCas13d.

The terms “guide RNA” or “gRNA” as used herein may be used interchangeably and refer to a polynucleotide which facilitates the specific targeting of a CRISPR-associated endonuclease or a modified CRISPR-associated endonuclease to a target sequence such as a genomic or episomal sequence in a cell. According to some embodiments, gRNAs can be chimeric/uni-molecular (comprising a single RNA molecule, also referred to as single guide RNA or sgRNA) or modular (comprising more than one separate RNA molecule, typically a crRNA and tracrRNA which may be linked, for example by duplexing). According to some embodiments, a gRNA is an sgRNA.

The sgRNA is an RNA molecule which includes both the tracrRNA and crRNA (and a connecting loop). The sgRNA comprises a nucleotide sequence encoding the target homologous sequence (crRNA—CRISPR RNA) and the endogenous bacterial RNA that links the crRNA to the Cas nuclease (tracrRNA—trans-activating CRISPR RNA) in a single chimeric transcript. The variable region of the crRNA confers the cutting specificity of the associated endonuclease and is typically 20 nucleotides in length, but can be between about 17 to 20 nucleotides in length. The gRNA/Cas complex is recruited to the target sequence by the base-pairing between the gRNA sequence and the complement genomic DNA. For successful binding of Cas, the genomic target sequence must also contain the correct Protospacer Adjacent Motif (PAM) sequence immediately following the target sequence. The binding of the gRNA/Cas complex localises the Cas to the genomic target sequence so that the Cas can cut both strands of the DNA causing a double-strand or double-stranded break. Just as with ZFNs and TALENs, the double-stranded breaks produced by CRISPR/Cas can be repaired by HR (homologous recombination) or NHEJ (non-homologous end-joining), and are susceptible to specific sequence modification during DNA repair. The Cas nuclease has two functional domains: RuvC and HNH, each cutting a different DNA strand. When both of these domains are active, the Cas causes double strand breaks in the genomic DNA. A significant advantage of CRISPR/Cas is the high efficiency of this system coupled with the ability to easily create synthetic gRNAs. This creates a system that can be readily modified to target different genomic sites and/or to target different modifications at the same site. Additionally, protocols have been established which enable simultaneous targeting of multiple genes. However, apparent flexibility in the base-pairing interactions between the gRNA sequence and the genomic DNA target sequence allows imperfect matches to the target sequence to be cut by Cas.

The agents used in the methods of the invention may comprise one or multiple guide RNAs, in particular sgRNAs for each target. In preferred embodiments, a single guide RNA or sgRNA is used for introducing mutations in the endogenous ALS gene. In some embodiments, two or more guide RNAs or sgRNAs are used for introducing desired mutations in the target sequence.

In certain embodiments, separate CRISPR guide RNAs are used in the agents of the invention.

Modified versions of the Cas enzyme containing a single inactive catalytic domain, either RuvC- or HNH-, are called ‘nickases’. With only one active nuclease domain, the Cas nickase cuts only one strand of the target DNA, creating a single-strand break or “nick”. A single-strand break or single-stranded break, or nick, is mostly repaired by single strand break repair mechanism involving proteins such as but not only, PARP (sensor) and XRCCl/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas nickase are treated as a double-strand break, in what is often referred to as a “double nick” CRISPR system. A double-nick which is basically non-parallel DSB can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas nickase to create a double-nick by designing two gRNAs with target sequences in close proximity and on opposite strands of the genomic DNA would decrease off-target effect as either gRNA alone will result in nicks that are not likely to change the genomic DNA, even though these events are not impossible.

As used herein, a “modified CRISPR-associated endonuclease” (or “modified Cas”) refers to a Cas in which the catalytic domain has been altered and/or which are fused to additional domain. According to some embodiments, a “modified Cas” refers to a Cas which contains inactive catalytic domains (dead Cas, or dCas) and has no nuclease activity while still being able to bind to DNA based on gRNA specificity. According to some embodiments, a “modified Cas” refers to a Cas which has a nickase activity (“nCas9”), thus inducing a single strand break. In some embodiments, the modified CRISPR-associated endonuclease is a “modified Cas9 endonuclease”, possibly a catalytically inactive Cas9 (or “dCas9”) or a nickase Cas9 (“nCas9”). The dCas can be utilised as a platform for DNA transcriptional regulators to activate or repress gene expression by fusing the inactive enzyme to known regulatory domains. For example, the binding of dCas alone to a target sequence in genomic DNA can interfere with gene transcription. There are a number of publicly available tools available to help choose and/or design target sequences as well as lists of bioinformatically determined unique gRNAs for different genes in different species such as the Feng Zhang lab's Target Finder, the Michael Boutros lab's Target Finder “E-CRISP”, the RGEN Tools: “Cas-OFFinder”, the CasFinder: Flexible algorithm for identifying specific Cas9 targets in genomes and the CRISPR Optimal Target Finder.

In order to use the CRISPR/Cas system, both the guide RNAs and base editor or endonuclease should be introduced to the target cell or delivered as a ribonucleoprotein complex. According to some embodiments, the base editor/Cas/modified Cas and at least one gRNA are provided to a banana cell by introducing one or more vectors which express the base editor/Cas/modified Cas and the gRNAs. The vector can contain multiple cassettes on a single plasmid, or the cassettes are expressed from multiple separate plasmids. In certain embodiments, a first plasmid encodes a guide specific for the target gene and a base editor, and a second plasmid encodes a guide specific for an ALS gene (ALS1 or ALS2, or both) and a base editor. CRISPR plasmids are commercially available (such as the px330 plasmid from Addgene). The use of clustered regularly interspaced short palindromic repeats (CRISPR)-associated (Cas)-guide RNA technology and a Cas endonuclease for modifying plant genomes is also at least disclosed by Svitashev et al. (2015), Plant Physiology, 169 (2): 931-945; Kumar and Jain, 2015, Journal of Experimental Botany, 66: 47-57; and in U.S. Patent Application Publication No. 20150082478.

In certain embodiments, the agents operable to introduce the mutations are constructs present on one or more, such as two, plasmids. Accordingly, the agents operable to introduce the mutations in the ALS sequence and the agents operable to introduce the mutations in the target sequence may be encoded by sequence of a single plasmid, or the sequences may be divided over multiple plasmids.

### Transformation

The methods of the invention comprise contacting a banana cell or population of cells with agents operable to introduce at least one desired mutation in a target sequence and a mutation in an endogenous ALS gene. Any appropriate method of transforming the banana cell may be used. “Contacting” therefore encompasses any appropriate method for introducing agents, such as nucleic acid constructs, into a cell, including methods known as transformation, transfection and transduction.

In preferred embodiments, the agents comprise one or more nucleic acid constructs, such as nucleic acid constructs that encode guide RNAs and base editors or endonucleases. In such embodiments a banana cell or population of banana cells is transformed with the one or more nucleic acid constructs. Nucleic acid constructs useful in the embodiments of the invention may be constructed using recombinant technology well known to a person skilled in the art. Such nucleic acid constructs may be commercially available, suitable for transforming into plants and suitable for expression of the agents (such as guide RNAs and base editors or endonucleases) in the transformed cells.

In certain embodiments the banana cell that is transformed is a protoplast. In preferred embodiments, the banana cell that is transformed is an embryonic cell, such as a cell in an embryogenic cell suspension.

The nucleic acid constructs will generally comprise a promoter. As used herein, the “promoter” is plant-expressible, i.e. capable of inducing, conferring, activating or enhancing expression in a plant cell, tissue or organ. Examples of promoters useful in the methods of the invention include, but are not limited to, Actin, CANV 35S, CaMV19S, GOS2. A preferred promoter is CsVMV. The nucleotide sequences of the agents to be expressed may be optimised for plant expression. Examples of such sequence modifications include, but are not limited to, an altered G/C content to more closely approach that typically found in banana, and the removal of codons atypically found in the plant species commonly (referred to as codon optimisation). Banana plant cells may be transformed stably or transiently with the DNA constructs of the embodiments of the invention. Preferably the transformation is transient, at least in some of the banana cells and plants that are generated, so no heterologous DNA is integrated. In some embodiments, the promoter in the DNA construct comprises a Pol3 promoter. Examples of Pol3 promoters include, but are not limited to. AtU6-29, AtU626, AtU3B, AtU3d, and TaU6. In some embodiments, the promoter in the DNA construct comprises a Pol2 promoter. Examples of Pol2 promoters include, but are not limited to, CaMV 35S, CaMV 19S, ubiquitin, CVMV. In some embodiments, the promoter in the DNA construct comprises a 35S promoter. In some embodiments, the promoter in the DNA construct comprises a U6 promoter. In some embodiments, the promoter in the DNA construct comprises a Pol3 promoter (such as U6) operatively linked to the nucleic acid agent encoding at least one gRNA and/or a Pol2 promoter (such as CamV35S) operatively linked to the nucleic acid sequence encoding the CRISPR-associated endonuclease or base editor. In some embodiments, the promoter is CsVMV. In certain embodiments, multiple promoters are used. The DNA construct may be useful for transient expression by Agrobacterium-mediated transformation (Helens et al. (2005), Plant Methods 1: 13). In some embodiments, the nucleic acid sequences comprised in the DNA construct are devoid of sequences which are homologous to the genome of the banana plant cell (other than any guide sequences), so as to avoid integration into the banana genome.

Various cloning kits can be used in the context of the invention. As used herein, the “DNA construct” may be a binary vector. Examples of binary vectors are piCSL4723, pBIN19, pBHO1, pBinAR, pGPTV, pCAMBIA, pBIB-HYG, pBecks, pGreen or pPZP (Hajukiewicz, P. et al., Plant Molecular Biology, 25, 989 (1994), and Hellens et al. Trends in Plant Science 5, 446 (2000)). Examples of other vectors that can be used in the context of the present invention in various methods of DNA delivery (e.g. transfection, electroporation, particle bombardment, and viral inoculation) are: pGE-sgRNA (Zhang et al. Nature Communications 2016 7: 12697), pJIT163-Ubi-Cas9 (Wang et al. Nature Biotechnology 2004, 32, 947-951), pICH47742::2x35S-5′UTR-hCas9(STOP)-NOST (Belhan et al. Plant Methods 2013, 11; 9(l): 39).

In other embodiments, said agents are provided to the banana plant cell in RNA form. In yet other embodiments, the base editor or endonuclease is provided to the banana plant cell in protein form and the one or more guide RNAs are provided to said banana plant cell in RNA form. In some embodiments, the agents are provided to the banana plant cell as a ribonucleoprotein complex.

In other embodiments, said agents are provided to the banana plant cell in DNA form.

There are a number of methods of introducing (contacting) DNA, RNA, peptides and/or proteins or combinations of nucleic acids and peptides into plant cells. These include, for example, protoplast transformation (U.S. Pat. No. 5,508,184); desiccation/inhibition-mediated DNA uptake (Potrykus et al. (1985) Mol. Gen. Genet. 199: 183-8); electroporation (U.S. Pat. No. 5,384,253); agitation with silicon carbide fibres (U.S. Pat. Nos. 5,302,523 and 5,464,765); Agrobacterium-mediated transformation (U.S. Pat. Nos. 5,563,055, 5,591,616, 5,693,512, 5,824,877, 5,981,840, and 6,384,301); acceleration of DNA-coated particles (U.S. Pat. Nos. 5,015,580, 5,550,318, 5,538,880, 6,160,208, 6,399,861, and 6,403,865), acceleration of RNA-coated particles, and nanoparticles, nanocarriers and cell penetrating peptides (WO201126644A2; WO2009046384A1; WO2008148223A1). Other methods of transfection include the use of transfection reagents (e.g. Lipofectin, ThermoFisher), dendrimers (Kukowska-Latallo, J. F. et al. (1996), Proc. Natl. Acad. Sci. USA 93, 4897-902), cell penetrating peptides (Mae et al. (2005), “Internalisation of cell-penetrating peptides into tobacco protoplasts”, Biochimica et Biophysica Acta 1669(2): 101-7) or polyamines (Zhang and Vinogradov (2010), “Short biodegradable polyamines for gene delivery and transfection of brain capillary endothelial cells”, J Control Release, 143(3):359-366).

In some embodiments of the invention, the nucleic acid agents are provided to the banana plant cell using particle bombardment or biolistics. In preferred embodiments, the nucleic acid agents are provided to the banana plant cell using Agrobacterium transformation. Suitable protocols are described in the examples. In yet other embodiments, the nucleic acid agents are provided to the banana plant cell using protoplast transfection. In yet other embodiments, the nucleic acid agents are provided to the banana plant cell using electroporation. In yet other embodiments, the nucleic acid agents are provided to the banana plant cell using nanoparticle-mediated transfection.

The Examples demonstrate that Agrobacterium-mediated transformation is effective for introducing mutations in banana, and can be more efficient and effective than other transformation methods including particle bombardment. The Agrobacterium system includes the use of plasmid vectors that contain defined DNA segments. Preferably, the agents used in the invention in Agrobacterium-mediated transformation comprise one or more T-DNA constructs. T-DNA constructs and other constructs used in Agrobacterium-mediated transformation may integrate into the genome, but may also be expressed transiently. In preferred embodiments, the vector used in the invention is suitable for transient expression of the agents for introducing the mutations, and may be suitable for transient expression and integration. This allows the generation of some embryos or plants that are successfully mutated at the target sequence and endogenous ALS gene and that are non-transgenic, as shown in the Examples.

Preferably, the vector, for example the vector used in Agrobacterium-mediated transformation, additionally comprises a selectable marker, which may aid selection of embryos or plants that do not comprise the vector integrated into their genome. Suitable markers may include an antibiotic selection marker. Examples of antibiotic selection markers that can be used are, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes. Further preferred markers include: mutant psbA that provides triazine-resistance, in particular G264S and 1219V; and mutant enolpyruvylshikimate-3-phosphate synthase (EPSPS) that confer resistance to EPSP synthase inhibitors, in particular tryptophan 102 mutations, alanine 103 mutations and proline 106 mutations. In preferred embodiments, the selectable marker is a fluorescent protein, which is shown to be particularly effective in the Examples, such as mCherry, mTurquoise 2, GFP, such as sfGFP or pH-tdGFP, Gamillus, mNeonGreen, mEYPF, mCitrine, Citrine, or TagRFP. Preferably the selectable marker is mCherry.

A preferred Agrobacterium tumefaciens for use in the invention is AGL1.

Any suitable method for transforming banana cells with Agrobacterium carrying vectors may be used. In preferred embodiments a suspension of Agrobacterium cells is mixed with a banana ECS. The mixture may then be incubated and then the bacteria suspension can be removed to provide ECS cells that can be cultured. In certain embodiments, a heat shock treatment is applied (for example at 45 C for 5 minutes). Suitable methods are described in, for example, Shivani and Tiwari, Scientia Horticulturae, 246:675-685).

### Mutation in an Endogenous Acetolactate Synthase Gene

The invention utilises co-editing of target sequences and endogenous ALS genes in combination with ALS inhibitor selection to provide a highly efficient and effective strategy for editing banana genes. Accordingly, the invention concerns mutations in endogenous synthase genes that provide resistance to an ALS inhibitor.

The ALS genes encode proteins which functions in the branched-chain amino acid biosynthetic pathway. ALS proteins are irreversibly competitively inhibited by several herbicides from the sulfonylurea family (Garcia et al., 2017 PNAS, 114(7), E1091-E1100). Several ALS amino acid substitutions have been identified, both naturally occurring and artificially generated, that increase tolerance to sulfonylurea compounds (Yu & Powles, 2014a, Pest Management Science, 70(9)). These are typically all amino acid residues that form the active site pocket and alter the binding potential of the herbicide compound (Boutsalis et al., 1999, Pesticide Science, 55(5), 507-516).

Generally, a mutation in an endogenous ALS gene introduced according to the invention will be a substitution that alters the amino acid encoded by a codon and thereby results in an amino acid substitution in the ALS protein sequence. Exemplary amino acid substitutions that can be used in accordance with the invention to provide resistance to ALS inhibitors are provided in Table 1 below, with reference to the amino acid numbering according to Arabidopsis ALS (adapted from Yu & Powles, 2014, Pest Management Science, 70(9). The Arabidopsis ALS amino acid sequence can be aligned to the banana ALS1 and ALS2 amino acid sequences to identify the corresponding residues. The banana ALS1 and ALS2 amino acid sequence can be compared to the banana ALS1 and ALS2 gene sequences to identify the nucleotides that can be altered to alter the encoded amino acid sequence.

TABLE 1

| | Herbicide | | Resistance- | Resistance- |
|---|---|---|---|---|
| | binding in | | endowing amino | endowing amino |
| | Arabidopsis | | acid substitutions | acid substitutions, |
| Amino acid | ALS | | identified in | artificially |
| and position | SU | IMI | resistant weeds | generated |
| Gly-121 | X | | | Ala, Asp, Asn, Ser |
| Ala-122 | X | X | Thr, Tyr, Val | Arg, Asn Asp, Cys, Glu, Gln. His, |
| | | | | Ile, Leu, Lys, Met, Phe, Pro, Ser, Thr, |
| | | | | Trp Tyr Val |
| Met-124 | | | | Glu, Ile |
| Arg142 | | | | Lys |
| Val-196 | X | | | Met |
| Pro-197 | X | | Ala, Arg, Asn, Cys, Gln, Glu, | Ala, Arg, Cys, Gln, Glu, Gly, Ser, |
| | | | Gly, His, Met, Ser, Trp, Tyr | Trp, Tyr, Val |
| Arg-199 | | X | | Ala, Glu |
| Met-200 | X | X | | |
| Ala-205 | X | | Val | Arg, Asn, Asp, Cys, Glu, Thr, Trp, |
| | | | | Tyr, Val |
| Phe-206 | X | X | | Ala, His, Trp, Tyr |
| Gln-207 | X | X | | |
| Lys-256 | X | X | | Asp, Asn, Gly, Pro Thr |
| Met-351 | X | X | | Cys, Gln, Gly, Lys, Pro, Tyr, Val |
| His-352 | | | | Gln, Met, Phe |
| Arg-373 | | | | Phe |
| Asp-375 | | | | Ala, Glu |
| Asp-376 | X | X | | Glu Ala, Asn, Cys, Glu, Gly, Pro, Ser, |
| | | | | Trp, Val |
| Arg-377 | X | X | His | Lys |
| Met-570 | X | | | Ala, Asn, Cys |
| Val-571 | X | | | Ala, Asn, Cys, Gln, Ile, Ser, Trp |
| Trp-574 | X | X | Arg, Gly, Met, Leu | Ala, Arg, Asp, Asn, Cys, Gln, Glu, |
| | | | | Gly, His, Ile, Leu, Lys, Met, Phe, Ser, |
| | | | | Thr, Tyr, Val |
| Phe-578 | | | | Arg, Asp, Asp, Cys, Glu, Gly, Ile, |
| | | | | Leu, Lys, Pro, Ser, Trp |
| Ser-653 | X | X | Asn, Thr, Ile | Asn, Phe, Thr |
| Gly-654 | | X | Glu, Asp | Glu |

SU = sulfonylurea,
IMI = imidazolinone

Preferably, a mutation in an endogenous ALS gene introduced according to the invention is a substitution that causes a substitution in the encoded amino acid sequence. Preferred amino acid substitutions are at residues Gly-121, Ala-122, Met-124, Arg142, Val-196, Pro-197, Arg-199, Met-200, Ala-205, Phe-206, Gln-207, Lys-256, Met-351, His-352, Arg-373, Asp-375, Asp-376, Arg-377, Met-570, Val-571, Trp-574, Phe-578, Ser-653, Gly-654, numbered according to Arabidopsis ALS. Certain substitutions ablate binding to sulfonylurea and/or imidazolinone herbicides, as shown in Table 1. For optimum performance, the appropriate herbicide should be used for selection.

Preferred residues to mutate are Pro-197, Trp-574-Leu and Ala-121, numbered according to the Arabidopsis ALS sequence.

Preferably, a mutation in an endogenous ALS gene introduced according to the invention results in a substitution of the residue corresponding to Pro-197 in the Arabidopsis ALS sequence, which is Pro-187 in banana ALS1 and Pro-181 in ALS2. Most preferably, the introduced substitution(s) comprise(s) Pro187Ser in ALS1 and/or Pro181Ser in ALS2. Suitable guide RNAs for introducing these mutations comprise SEQ ID NO:19 and SEQ ID NO:20, or a sequence having at least 90, 95, 98 or 99% sequence identity to SEQ ID NO:19 or SEQ ID NO:20. In certain embodiments, the introduced substitution(s) comprise(s) Pro187Phe in ALS1 and/or Pro181Phe in ALS2, which is also expected to provide resistance to an ALS inhibitor. In certain embodiments, the banana cell or plant obtained by the method of the invention is heterozygous for more than one mutation at ALS1 and/or more than one mutation ALS2, and the introduced substitutions comprise Pro187Ser and Pro187Phe in ALS1 and/or comprise Pro181Ser and Pro181Phe in ALS2.

In certain embodiments, the method comprises introducing a mutation, such as at the codon encoding the residue corresponding to Pro-197 in the Arabidopsis ALS sequence, in the endogenous ALS1 gene. Such embodiments generally will involve use of a guide RNA targeted to ALS1. In certain embodiments, the method comprises introducing a mutation, such as at the codon encoding the residue corresponding to Pro-197 in the Arabidopsis ALS sequence, in the endogenous ALS2 gene. Such embodiments generally will involve use of a guide RNA targeted to ALS2. In certain embodiments, the method comprises introducing a mutation, such as at the codon encoding the residue corresponding to Pro-197 in the Arabidopsis ALS sequence, in both the endogenous ALS1 and ALS2 genes. Such embodiments may generally involve use of separate guide RNAs targeted to ALS1 and ALS2, but may involve use of a single guide RNA capable of targeting both ALS1 and AL2. In certain embodiments, the method does not comprise introducing a mutation in the endogenous ALS1 gene, and for example, the agent does not comprise any sequence such as a guide RNA targeted to the endogenous ALS1 gene.

The method of the invention may involve introducing mutations at one, two or three alleles of the endogenous ALS genes. In some embodiments, the banana cell, tissue or plant obtained by the method of the invention is homozygous for mutations at ALS1, ALS2, or ALS1 and ALS2. In some embodiments, the banana cell or plant obtained by the method of the invention is heterozygous for mutations at ALS1, ALS2, or ALS1 and ALS2.

Accordingly, it is not necessary that the mutation in the ALS gene introduced in the method of the invention provides complete resistance to ALS inhibitors such that treatment with ALS inhibitors has no effect. In contrast, banana embryos and plants generated according to the method may exhibit delayed development or reduced growth upon treatment with an ALS inhibitor. However, the mutation in the ALS gene will nevertheless provide resistance to treatment with ALS inhibitors because banana embryos and plants generated according to the method will exhibit improved development or growth relative to wild type banana embryos and plants, under treatment with an ALS inhibitor.

### Desired Mutation in a Target Sequence

The invention provides an improved method for generating banana embryos and plants comprising at least one desired mutation in one or more target sequences.

A “mutation”, as used herein, can mean at least one nucleotide insertion, at least one nucleotide deletion, an insertion-deletion (indel), an inversion, at least one nucleotide substitution, or any combination of the foregoing. The modification can result in a frameshift, a missense mutation, loss-of-function mutation, a nonsense mutation or a gain-of-function mutation in the target sequence. The agents operable to introduce the desired mutation can be designed according to the type of mutation that is required, using standard techniques.

In some embodiments, the target sequence encodes a protein involved in banana ripening, such as a protein involved in the production of ethylene. In some embodiments, sequences encoding ACO (1-Aminocyclopropane-1-Carboxylic Acid Oxidase) and/or ACS (ACC-synthase) may be targeted. ACO and ACS are involved in the production of ethylene. In certain embodiments, desired mutations reduce expression or activity of ACO and/or ACS, which may delay ripening of the banana fruit. In preferred embodiments, a desired mutation introduces a stop codon in the sequence encoding ACO or ACS or both. In preferred embodiments, a target sequence is ACO1 (for example, SEQ ID NO:46, or a sequence having at least 90, 95, 98 or 99% sequence identity to SEQ ID NO:46). In some such embodiments, the one or more guide RNAs used to introduce a mutation comprise a sequence selected from the group consisting of: SEQ ID NO:24-26, or a sequence having at least 90, 95, 98 or 99% sequence identity to SEQ ID NO:24-26. In certain embodiments, the target is ACO and the target sequence is selected from SEQ ID NO:46 and SEQ ID NO:47, or a sequence having at least 90, 95, 98 or 99% sequence identity to SEQ ID NO:46 or SEQ ID NO:47. In certain embodiments, the target is ACS and the target sequence is selected from SEQ ID NO:43, SEQ ID NO:44 and SEQ ID NO:45, or a sequence having at least 90, 95, 98 or 99% sequence identity to SEQ ID NO:43, SEQ ID NO:44 or SEQ ID NO:45.

In some embodiments, the target sequence encodes a protein involved in banana browning. In some embodiments, a target sequence is the sequence encoding PPO (polyphenol oxidase). PPO is the enzyme thought to be responsible for browning in banana. In preferred embodiments, a target sequence is PPO2 (for example, SEQ ID NO:48, or a sequence having at least 90, 95, 98 or 99% sequence identity to SEQ ID NO:48). In certain embodiments, a target sequence is PPO1 (for example, SEQ ID NO:49, or a sequence having at least 90, 95, 98 or 99% sequence identity to SEQ ID NO:49). In certain embodiments, a target sequence is PPO3 (for example, SEQ ID NO:50, or a sequence having at least 90, 95, 98 or 99% sequence identity to SEQ ID NO:50). In certain embodiments, a target sequence is PPO4 (for example, SEQ ID NO:51, or a sequence having at least 90, 95, 98 or 99% sequence identity to SEQ ID NO:51). In certain embodiments, a target sequence is PPO5 (for example, SEQ ID NO:52, or a sequence having at least 90, 95, 98 or 99% sequence identity to SEQ ID NO:52). In certain embodiments, a target sequence is PPO6 (for example, SEQ ID NO:53, or a sequence having at least 90, 95, 98 or 99% sequence identity to SEQ ID NO:53). In certain embodiments, a target sequence is PPO7 (for example, SEQ ID NO:54, or a sequence having at least 90, 95, 98 or 99% sequence identity to SEQ ID NO:54). In certain embodiments, a target sequence is PPO8 (for example, SEQ ID NO:55, or a sequence having at least 90, 95, 98 or 99% sequence identity to SEQ ID NO:55). In certain embodiments, a target sequence is PPO9 (for example, SEQ ID NO:56, or a sequence having at least 90, 95, 98 or 99% sequence identity to SEQ ID NO:56). In certain embodiments, a desired mutation reduces expression or activity of PPO, which may delay ripening of the banana fruit. In preferred embodiments, a desired mutation introduces a stop codon in the sequence encoding PPO. In some such embodiments, the one or more guide RNAs used to introduce a mutation comprise a sequence selected from the group consisting of: SEQ ID NO:21-23, or a sequence having at least 90, 95, 98 or 99% sequence identity to SEQ ID NO:21-23.

Optionally multiple sequences may be targeted for mutation (e.g. any combination of ACO, ACS and PPO genes could be targeted simultaneously) and, for each sequence target, one or a plurality of mutations could be introduced. The present invention can be used to mutate a great range of different genes to achieve various effects. The invention is particularly useful for modulation of endogenous genes to provide improved traits and to protect organisms against different biotic and abiotic stresses such as e.g. cancer, viruses, insects, fungi, nematodes, heat, drought, starvation etc. This section provides preferred target genes into which mutations are introduced according to the invention. Preferably, the desired mutation reduces the expression of the gene or the activity of the expressed protein. Preferably, the mutations is the introduction of a stop codon.

In certain embodiments, mutating a gene according to the present invention provides a plant with increased stress tolerance, increased yield, increased growth rate or increased yield quality. In certain embodiments, the target sequence encodes a protein involved in stress tolerance, yield, growth rate or yield quality.

The phrase “stress tolerance” as used herein refers to the ability of a plant to endure a biotic or abiotic stress without suffering a substantial alteration in metabolism, growth, productivity and/or viability.

The phrase “abiotic stress” as used herein refers to the exposure of a plant, plant cell, or the like, to a non-living (“abiotic”) environmental, physical or chemical agent that has an adverse effect on metabolism, growth, development, propagation, or survival of the plant (collectively, “growth”). An abiotic stress can be imposed on a plant due, for example, to an environmental factor such as water (e.g., flooding, drought, or dehydration), anaerobic conditions (e.g., a lower level of oxygen or high level of CO₂), abnormal osmotic conditions (e.g. osmotic stress), salinity, or temperature (e.g., hot/heat, cold, freezing, or frost), an exposure to pollutants (e.g. heavy metal toxicity), anaerobiosis, nutrient deficiency (e.g., nitrogen deficiency or limited nitrogen), atmospheric pollution or UV irradiation.

The phrase “biotic stress” as used herein refers to the exposure of a plant, plant cell, or the like, to a living (“biotic”) organism, yet including viruses, that has an adverse effect on metabolism, growth, development, propagation, yield or survival of the plant (collectively, “growth”). Biotic stress can be caused by, for example, bacteria, viruses, fungi, parasites, beneficial and harmful insects, weeds, and cultivated or native plants.

The phrase “yield” or “plant yield” as used herein refers to increased plant growth (growth rate), increased crop growth, increased biomass, and/or increased plant product production (including grain, fruit, seeds, etc.).

According to one embodiment, in order to generate a plant with increased stress tolerance, increased yield, increased growth rate or increased yield quality, the desired mutation is in a gene of the plant conferring sensitivity to stress, decreased yield, decreased growth rate or decreased yield quality. In such embodiments, the mutation may inactivate the gene or reduce its expression or activity.

Any method known in the art for assessing increased stress tolerance may be used in accordance with the present invention. Exemplary methods of assessing increased stress tolerance include, but are not limited to, downregulation of PagSAP1 in poplar for increased salt stress tolerance as described in Yoon, S K., Bae, E K., Lee, H. et al. Trees (2018) 32: 823.), and increased drought tolerance in tomato by downregulation of SlbZIP38 (Pan Y et al. Genes 2017, 8, 402; doi:10.3390/genes8120402.

Any method known in the art for assessing increased yield may be used in accordance with the present invention. Exemplary methods of assessing increased yield include, but are not limited to, reduced DST expression in rice as described in Ar-Rafi Md. Faisal, et al. AJPS>Vol. 8 No. 9, August 2017 DOI: 10.4236/ajps.2017.89149; and downregulation of BnFTA in canola resulted in increased yield as described in Wang Y et al., Mol Plant. 2009 January; 2(1): 191-200.doi: 10.1093/mp/ssn088.

Any method known in the art for assessing increased growth rate may be used in accordance with the present invention. Exemplary methods of assessing increased growth rate include, but are not limited to, reduced expression of BIG BROTHER in Arabidopsis or GA2-OXIDASE results in enhance growth and biomass as described in Marcelo de Freitas Lima et al. Biotechnology Research and Innovation(2017)1,14-25.

Any method known in the art for assessing increased yield quality may be used in accordance with the present invention. Exemplary methods of assessing increased yield quality include, but are not limited to, down regulation of OsCKX2 in rice results in production of more tillers, more grains, and the grains were heavier as described in Yeh S_Y et al. Rice (N Y). 2015; 8: 36; and reduce OMT levels in many plants, which result in altered lignin accumulation, increase the digestibility of the material for industry purposes as described in Verma SR and Dwivedi UN, South African Journal of Botany Volume 91, March 2014, Pages 107-125.

According to one embodiment, the method further enables generation of a plant comprising increased sweetness, increased sugar content, increased flavour, improved ripening control, increased water stress tolerance, increased heat stress tolerance, and increased salt tolerance. One of skill in the art will know how to utilise the methods described herein to choose target gene sequences for mutating.

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission.

According to one embodiment, the pest is an invertebrate organism.

Exemplary pests include, but are not limited to, insects, nematodes, snails, slugs, spiders, caterpillars, scorpions, mites, ticks, fungi, and the like.

Identification of plant or pathogen target genes to be mutated may be achieved using any method known in the art such as by routine bioinformatics analysis.

### Further Selection

In certain embodiments, the method comprises additional selection steps following treatment using an ALS inhibitor. In particular, it may be useful to select banana embryos or plants that do not comprise heterologous DNA integrated into their genome (such as nucleic acid constructs encoding the agents) and embryos or plants that are confirmed to comprise the at least one desired mutation in a target sequence.

In certain embodiments, the method comprises, following selection using an ALS inhibitor, selecting at least one banana cell or plant that comprises the at least one desired mutation in a target sequence. The mutation may be at least one nucleotide insertion; at least one nucleotide deletion: at least one nucleotide substitution; and any combination of the foregoing. The selection of at least one banana cell or plant that comprises the at least one desired mutation in a target sequence may comprise detecting the presence of the mutated genome sequence.

In preferred embodiments, treating the banana cell with the ALS inhibitor provides a population of banana cells and at least 1%, 5%, 10% or 15% of the cells in the population do not comprise heterologous DNA. In further preferred embodiments, at least 0.1% 1%, 2%, 3%, 4% or 5% of the cells comprise the mutation in the endogenous acetolactate synthase gene and comprise the desired mutation in the target sequence.

In certain embodiments, the method comprises, following selection using an ALS inhibitor, selecting at least one banana cell or plant that does not comprise any heterologous DNA, in particular integrated nucleic acid constructs that encode the agents operable to introduce mutations. The selection of such at least one banana cell or plant may comprise confirming the absence of the nucleic acid construct sequence in the genome of the cell or plant.

This can be achieved using any technique known in the art capable of detecting the modification or editing event, such as, but not limited to, DNA sequencing (e.g. next generation sequencing), electrophoresis, an enzyme-based mismatch-detection assay, and a hybridisation assay such as PCR, RT-PCR, RNase protection, in-situ hybridisation, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing. High-resolution melting analysis is another method of validating the presence of an editing event. Yet another method is the heteroduplex mobility assay. Mutations can also be detected by analysing re-hybridised PCR fragments directly by native polyacrylamide gel electrophoresis (PAGE). This method takes advantage of the differential migration of heteroduplex and homoduplex DNA in polyacrylamide gels. Other methods of validating the presence of editing events are described in length in Zischewski (2017), Biotechnology Advances 1(1):95-104.

In preferred embodiments, the agents used to transform the banana cell are nucleic acid constructs, such as T-DNA plasmids, and the constructs additionally encode a selectable marker, such as a fluorescent marker, such as mCherry. Such markers may aid in the selection of non-transgenic embryos, for example, that do not have the constructs integrated into their genome and only transiently expressed the editing agents, because they will not express the selectable marker, as demonstrated in the examples.

Suitable markers may include an antibiotic selection marker. Examples of antibiotic selection markers that can be used are, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes. Further preferred markers include: mutant psbA that provides triazine-resistance, in particular G264S and I219V; and mutant enolpyruvylshikimate-3-phosphate synthase (EPSPS) that confer resistance to EPSP synthase inhibitors, in particular tryptophan 102 mutations, alanine 103 mutations and proline 106 mutations. In preferred embodiments, the selectable marker is a fluorescent protein, which is shown to be particularly effective in the Examples, such as mCherry, mTurquoise 2, GFP, such as sfGFP or pH-tdGFP, Gamillus, mNeonGreen, mEYPF, mCitrine, Citrine, or TagRFP. Preferably the selectable marker is mCherry.

Banana Cells, Tissues and Plants As used herein, the term “banana” refers to a plant of the genus Musa, including plantains. These include Musa acuminata (e.g. Musa acuminata banksia, Musa acuminata Calcutta, and Musa acuminata DH-Pahang), Musa balbisiana, Musa itinerans, and autotriploid Musa acuminata ‘Cavendish’ and ‘Gros Michel’. According to preferred embodiments, banana is autotriploid Musa acuminata ‘Cavendish’. According to further preferred embodiments, the banana is the Grand Nain cultivar. Cultivated bananas are infertile autotriploids (AAA) derived from the progenitor species Musa acuminata (genome AA). Additionally, plantains (AAB or ABB) are infertile interspecific allotriploids derived from the hybridisation of Musa acuminata (AA) and Musa balbisiana (genome BB). The triploid nature of cultivated banana and plantain prevents them from producing viable seeds, whereas wild species are diploid and can produce viable seeds. In preferred embodiments, the banana plant is triploid. Other ploidies are contemplated, including diploid and tetraploid.

As used herein, the term “plant” refers to whole plants, grafted plants, ancestors and progeny of the plants, plant organs, plant tissues, and “plant parts”. “Plant parts”, as used herein, include differentiated and undifferentiated tissues including, but not limited to roots (including tubers), rootstocks, stems, scions, shoots, fruits, leaves, pollens, seeds, tumour tissue, and various forms of cells and culture (e.g. single cells, protoplasts, embryos, embryonic cells, and callus tissue). The plant tissue may be in plant or in a plant organ, tissue or cell culture.

In particular embodiments, the plant part is a fruit. Fruit comprises tissues such as fruit flesh and fruit peel. In other embodiments, the plant part is a seed. The term “seed” as used herein refers to a unit of reproduction of a flowering plant capable of developing into another such plant. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

A “transgenic plant” includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant.

A “heterologous” polynucleotide, as used herein, comprises a sequence that originates from a foreign species.

“Transgenic” can include any cell, cell line, callus, tissue, plant part or plant, the genotype of which has been altered by the presence of heterologous nucleic acid including those transgenics initially so altered as well as those created by sexual crosses or asexual propagation from the initial transgenic. In some plants, the heterologous polynucleotide which is introduced into the plant genome can be removed through breeding. This process is not possible in banana, as described hereinabove.

According to preferred embodiments, the banana cells, banana plants, or banana plant parts described herein and obtained by the methods of the invention are non-transgenic. According to some embodiments, the methods disclosed herein result in a banana cell, banana plant or banana plant cell which is non-transgenic.

The alterations of the genome (chromosomal or extra-chromosomal) by conventional plant breeding methods, by the genome editing procedure described herein (that does not result in an insertion of a foreign polynucleotide), or by naturally occurring events such as random cross-fertilisation, non-recombinant viral infection, non-recombinant bacterial transformation, non-recombinant transposition, or spontaneous mutation are not intended to be regarded as transgenic.

In some embodiments, the “banana plant” is of a banana breeding line, such as an elite line or purebred line, or a banana variety or breeding germplasm. The term “breeding line”, as used herein, refers to a line of a cultivated banana having commercially valuable or agronomically desirable characteristics, as opposed to wild varieties or landraces. The term includes reference to an “elite breeding line” or “elite line”, which represents an essentially homozygous, usually inbred, line of plants used to produce commercial F₁ hybrids. An “elite breeding line” is obtained by breeding and selection for superior agronomic performance comprising a multitude of agronomically desirable traits. An “elite plant” is any plant from an elite line. Superior agronomic performance refers to a desired combination of agronomically desirable traits as defined herein, wherein it is desirable that the majority, preferably all of, the agronomically desirable traits are improved in the elite breeding line as compared to a non-elite breeding line. Elite breeding lines are essentially homozygous and are preferably inbred lines. The term “elite line”, as used herein, refers to any line that has resulted from breeding and selection for superior agronomic performance.

The terms “cultivar” and “variety” are used interchangeably herein and denote a plant with has deliberately been developed by breeding, e.g. crossing and selection, for the purpose of being commercialised, e.g. used by farmers and growers, to produce agricultural products for own consumption or for commercialisation. The term “breeding germplasm” denotes a plant having a biological status other than a “wild” status, which “wild” status indicates the original non-cultivated, or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. As used herein, the terms “purebred”, “pure inbred” or “inbred” are interchangeable and refer to a substantially homozygous plant or plant line obtained by repeated selfing and/or backcrossing.

As used herein, the term “banana plant cell” is a cell of a banana plant. Banana plant cells include cells from seeds, suspension cultures, embryos, meristematic regions, callus tissue, leaves, roots, shoots, gametophytes, sporophytes, pollen, microspores, embryogenic cells, somatic cells, and protoplasts. Protoplasts can be derived from any plant tissue, such as, but not limited to, roots, leaves, embryogenic cell suspension, callus, or seedling tissue. According to some embodiments, a banana plant cell is a cell of an Embryogenic cell suspension (ECS).

The invention further provides a banana plant cell obtainable by any one of the foregoing methods of the invention.

In some embodiments of the invention, the method further comprises regenerating a banana plant from said banana plant cell.

As used herein, “regenerating” may comprise growing banana plant cells (which include protoplasts) into whole banana plants by first growing the banana plant cells into groups that develop into a callus, followed by the regeneration of shoots (caulogenesis) from the callus using plant tissue culture methods. The growth of banana protoplasts into callus and subsequent regeneration of shoots requires the proper balance of plant growth regulators in the tissue culture medium that must be customised. Protoplasts may also be used for plant breeding, using a technique called protoplast fusion. Protoplasts from different species are induced to fuse by using an electric field or a solution of polyethylene glycol. This technique may be used to generate somatic hybrids in tissue culture. Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment (see Maheshwari et al. (1986), “Differentiation of Protoplasts and of Transformed Plant Cells”: 3-36. Springer-Verlag, Berlin). The regenerated banana plants can be subjected to selection. The banana plant or cells thereof may be devoid of a transgene, i.e. “non-transgenic”. For example, the banana plants may be devoid of any of the DNA constructs encoding any of the CRISPR/Cas system as used in some of the embodiments of the invention.

Any appropriate media and culturing techniques may be used in the methods of the invention. Suitable exemplary media and techniques are provided in, for example, Banana and Plantain embryogenic cell suspensions—INIBAP Technical Guidelines 8 (Strosse et al., Vézina and Picq, eds, 2003). For example, a suitable embryo development medium (EDM) is MA3 in Strosse et al. and a suitable cell suspension medium (CS) is ZZ1 (see Appendix 2).

In some of embodiments, the method of the invention further comprises harvesting fruit from said banana plant. Each adult banana plant produces a single bunch, which is formed by many banana fruits or “fingers” and clustered in several “hands”. In this regard, “harvesting” has the conventional meaning. For example, banana bunches may be cut by hand (usually involving 2 or 3 people) using a sharp curved knife or a machete. The harvest usually occurs when the banana fruits are still green and firm, 7 to 14 days prior to ripening.

The invention further provides a banana plant or plant part obtainable by the foregoing method.

Yet further provided by the invention is fruit harvested from a banana plant obtainable by the foregoing method of the invention, wherein said fruit flesh and/or fruit peel is characterised by a phenotype of resistance to an ALS inhibitor and the phenotype provided by the desired mutation in the target sequence.

The invention further provides a banana plant or fruit comprising a nucleic acid sequence encoding a mutated ALS polypeptide and/or comprising a mutated ALS polypeptide, wherein the mutated ALS polypeptide comprises a sequence with at least 80, 85, 90, 95, 97, 98, 99 or 100% sequence identity to SEQ ID NO:9 and having a substitution of the proline at residue 187, preferably for serine, or wherein the mutated ALS polypeptide comprises a sequence with at least 80, 85, 90, 95, 97, 98, 99 or 100% sequence identity to SEQ ID NO:10 with a substitution of the proline at residue 181, preferably for serine. In further embodiments, the substitution is for phenylalanine.

### General

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended embodiments, the singular forms “a”, “an”, and “the” include plural referents unless the content clearly dictates otherwise. Thus, for example, reference to “a polypeptide” includes “polypeptides”, and the like.

Unless specifically prohibited, the steps of a method disclosed herein may be performed in any appropriate order and the order in which the steps are listed should not be considered limiting.

All publications, patents and patent applications cited herein are hereby incorporated by reference in their entirety.

### EXAMPLES

### Example 1: Chlorsulfuron (CSF) Kill Curves to Test Feasibility of Using CSF as a Herbicide for Selection

This example surprisingly demonstrates that it is possible to use an ALS inhibitor, specifically chlorsulfuron (CSF), to exert selection pressure on banana, including at the embryogenic cell stage in an embryogenic cell suspension, which is a non-green tissue. This example therefore demonstrates that ALS inhibitors can be used as a herbicide for banana, including as a selection agent.

### Kill Curves—Banana ECS

CSF concentrations were initially selected based on the following CSF concentrations, which have been reported to be effective in other plant species in the publications listed below:
1. Sugar cane callus—3.6, 20, 50, 200, 500 μg/L: 3.6 μg/L was the best concentration for selection after 5 weeks.
2. Potato somatic cells—20 μg/L
3. Tomato plants—3.6 μg/L
4. Rice protoplasts—3.6 μg/L for 10dd
5. Marchantia polymorpha spores—0.5 μM (180 μg/L) for 7dd
1. van der Vyver C, et al. 2013. In Vitro Cell Dev Biol—Plant 49:198-206.
2. Barrel P, et al. 2017. BMC Biotechnology 17:49.
3. Veillet F, et al. 2019. Int J Mol Sci 20:402.
4. Li Z., et al. 1992. Plant Physiol 100:662-668.
5. Ishizaki K, et al. 2015. PLoS ONE 10(9): e0138876.

### Kill Curves Setup in Banana ECS

Banana embryogenic cell suspensions (ECS) were pelleted in a falcon tube and mixed with CS (cell suspension) medium to a final cell density of 1-3%. An equal volume of the mixture was split into 6 flasks and the relevant amount of CSF was added to the final concentrations of 0, 10, 20, 50, 100 and 500 μg/L (1^{st} kill curve) or 0, 0.5, 5, 50 and 100 mg/L (2^{nd} kill curve). The ECS-CSF mixtures were placed/inoculated in 6 well plates (5 mL per well) and incubated at 65 rpm in the dark for 3, 5, 10, 15 days at 25° C.

At each time point, the ECS were collected in a cell strainer and washed with CS (cell suspension) medium to remove all the CSF. The cells were collected from the strainer and inoculated into embryo developing media (EDM) plates and developed for 2-3 months (until fully developed embryos were observed). The cells/embryos were subcultured every month. Each treatment was performed in triplicate and treated independently.

### Assessment of Number of Banana Embryos

The number of embryos was assessed 2-3 months after CSF treatment. The embryos were counted under a stereo microscope using a 50-square grid to help the counting. For plates with too many embryos, only 3 squares from the 50 were counted and then the number of embryos was normalised for all of the plate. For the other plates all embryos were counted. Mean values±standard deviation (Stdev) were obtained from 3 technical replicates.

### Kill Curves Setup in Plantlets

The effect of CSF was also evaluated in rooting plantlets. Three methods were tested, (i) incorporation of CSF in the rooting medium (RM), (ii) spraying the plantlets with CSF solution and (iii) incorporation in the media of a lower concentration of CSF (0.5 mg/L) and spraying the plantlets (concurrently).

Six CSF concentrations, 0, 0.05, 0.5, 5, 50 and 100 mg/L, were tested in rooting plantlets.

Rooting banana plantlets were cleared of roots and split from the corm to have single shoots for testing with RM medium with or without CSF. Five to six shoots with similar morphology were inoculated into each phytotray.

For spraying tests, CSF solutions (concentrations above) were prepared in sterile deionised water or in 0.015% Silwett L-77. One mL of solution was sprayed onto each phytotray (five shoots per phytotray, so 1 mL between five shoots). For control conditions (Omg/L CSF), 1 mL of deionised water was used.

### Results

### 1^{t} Kill Curve in Banana ECS

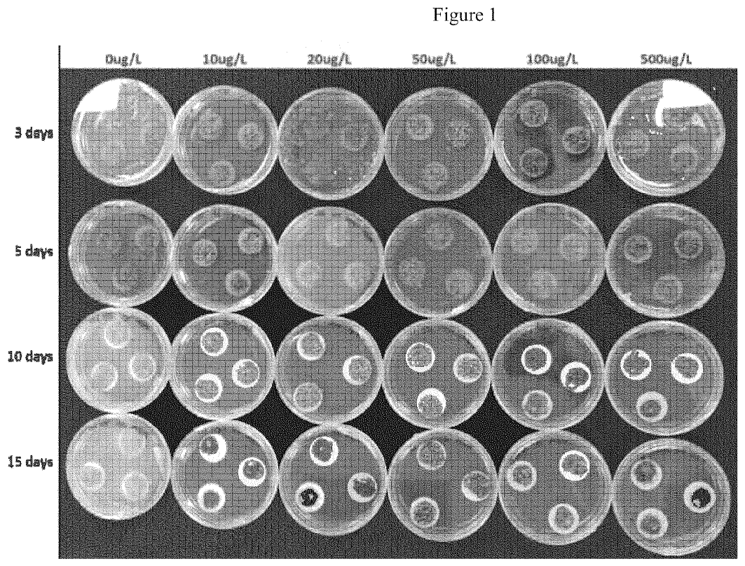

Concentrations tested—0, 10, 20, 50, 200, 500 μg/L. The results are shown in FIG. 1.

### 2Nd Kill Curve in Banana ECS

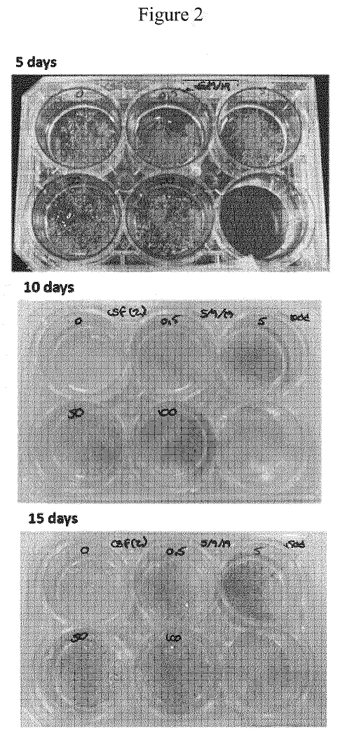

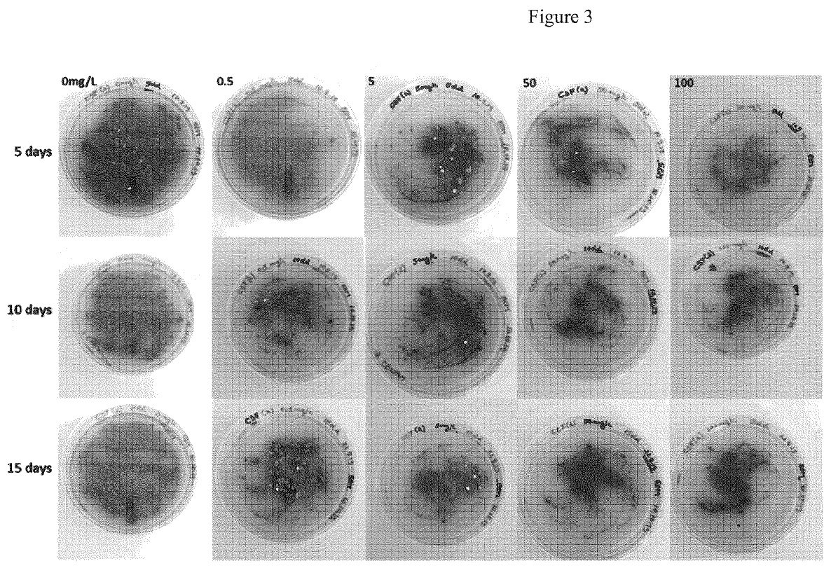

Concentrations tested—0, 0.5, 5, 50, 100 mg/L (200× more than 1^{st} kill curve). The results are shown in FIG. 2. FIG. 3 and Table 2.

TABLE 2
Number of banana embryos developed 3 months after a
short treatment (5, 10 and 15 days) with CSF (0, 0.5,
5, 50, 100 mg/L. Mean values ± standard deviation
(Stdev) were obtained from 3 technical replicates.

| | 5dd | | 10dd | | 15dd | |
|---|---|---|---|---|---|---|
| | Avg | stdev | Avg | stdev | Avg | stdev |
| Concentration | | | | | | |
| (mg/L) | | | | | | |
| 0 | 3163 | 619 | 4138 | 159 | 4842 | 1003 |
| 0.5 | 1109 | 627 | 127 | 31 | 217 | 77 |
| 5 | 127 | 22 | 18 | 5 | 29 | 17 |
| 50 | 58 | 15 | 9 | 9 | 4 | 1 |
| 100 | 57 | 24 | 8 | 5 | 14 | 1 |
| Fold-change | | | | | | |
| non-CSF/+CSF | | | | | | |
| 0.5 | 2.9 | | 32.6 | | 22.3 | |
| 5 | 24.9 | | 229.9 | | 167.0 | |
| 50 | 54.5 | | 459.8 | | 1210.5 | |
| 100 | 55.5 | | 517.3 | | 345.9 | |

Two CSF kill curves were performed in banana ECS and the number of embryos was assessed 2-3 months later. The main purpose of these experiments was to determine the minimal lethal dose for CSF, a concentration that would reduce 100× the number of embryos in comparison to the numbers attained in the absence of CSF (control). (The concentration that would reduce the number of surviving embryos down to one hundredth ( 1/100) of the number attained in control conditions.)

In the 1^{st} CSF kill curve, lower concentrations of CSF (0, 10, 20, 50, 200, 500 μg/L) were tested, based on previous references, for 3, 5, 10 and 15 days. Despite the different CSF concentrations and the different periods of exposure, major differences in embryo number were not observed at the concentrations tested, including in the extreme case of 15 days of exposure with 500 μg/L CSF.

The CSF concentrations used seemed to be inefficient for killing banana ECS. It was decided to increase the CSF concentrations for the 2^{nd} kill curve.

In the 2^{nd} CSF kill curve higher concentrations were tested: 0, 0.5, 5, 50 and 100 mg/L for 5, 10 and 15 days. After treatment with CSF colour changes in banana ECS were observed with concentrations higher than 5 mg/L. Higher concentrations of CSF negatively affected the number of embryos. The measure of success was met at 5 mg/L of CSF for 10 days. A decrease higher than 200× (a decrease to less than one two-hundredth ( 1/200) in the number of embryos was observed. Five days of treatment was revealed to be inefficient for killing banana cells and 15 days of treatment was not better than 10 days.

### Kill Curves in Banana Rooting Plantlets

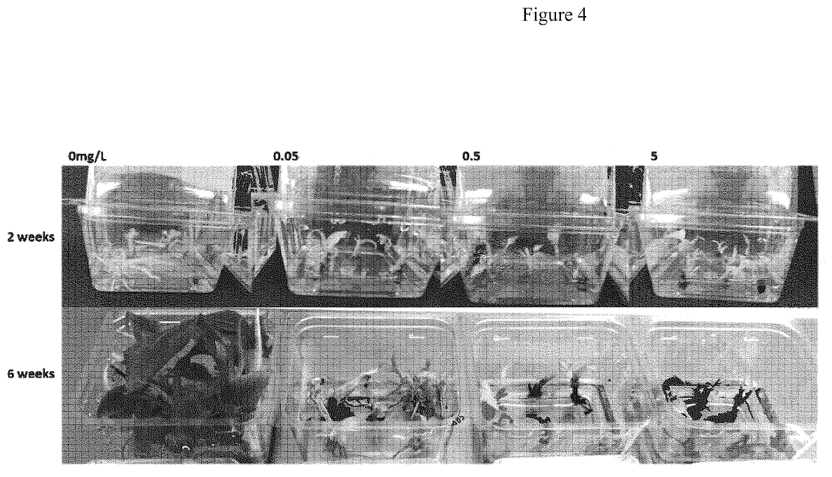

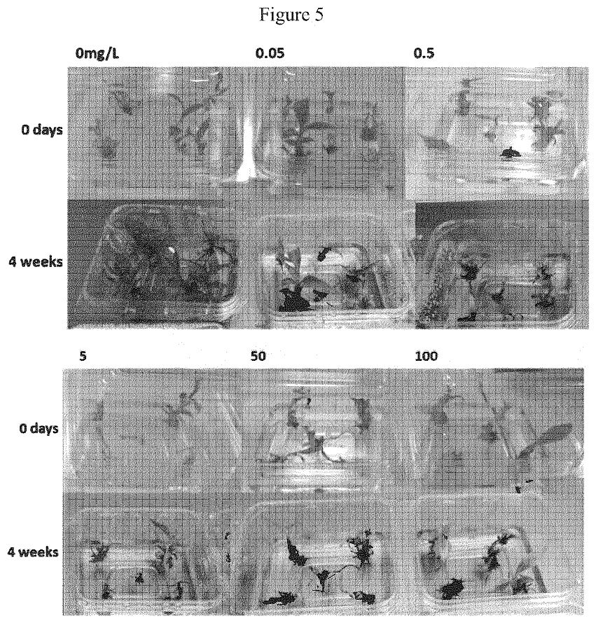

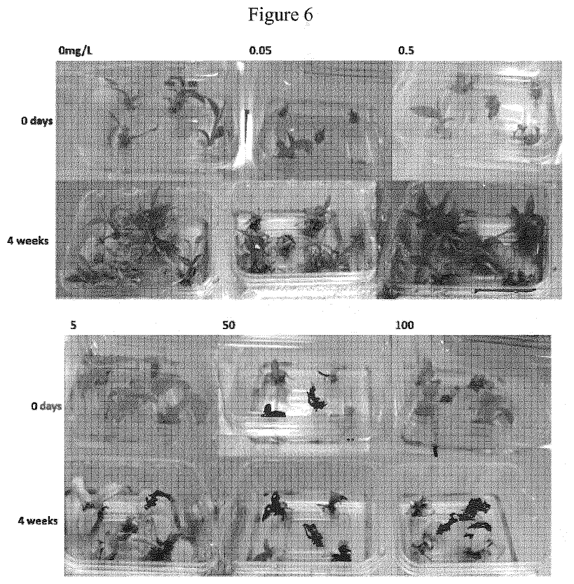

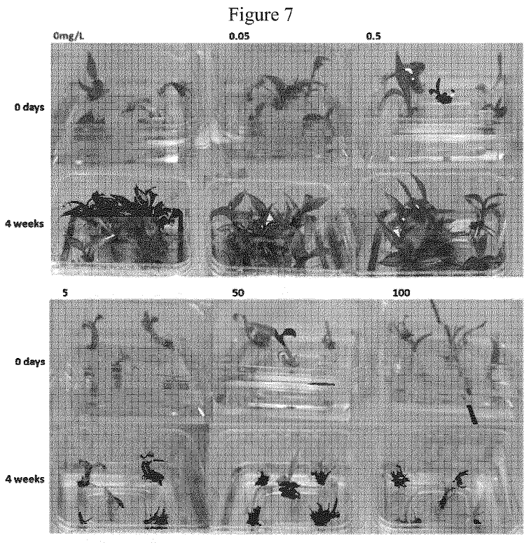

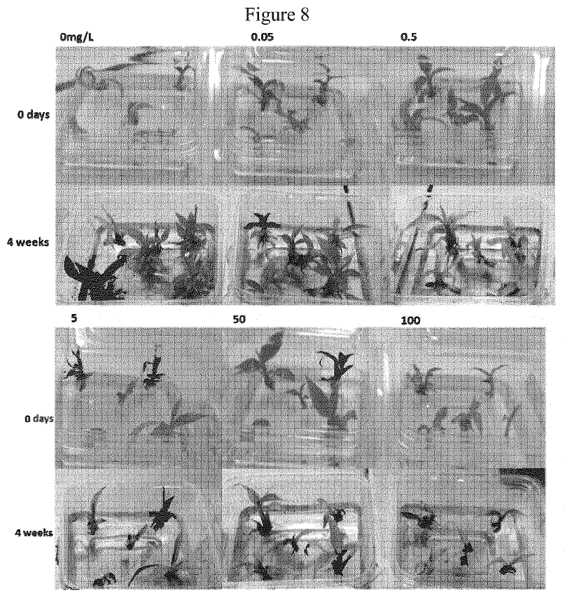

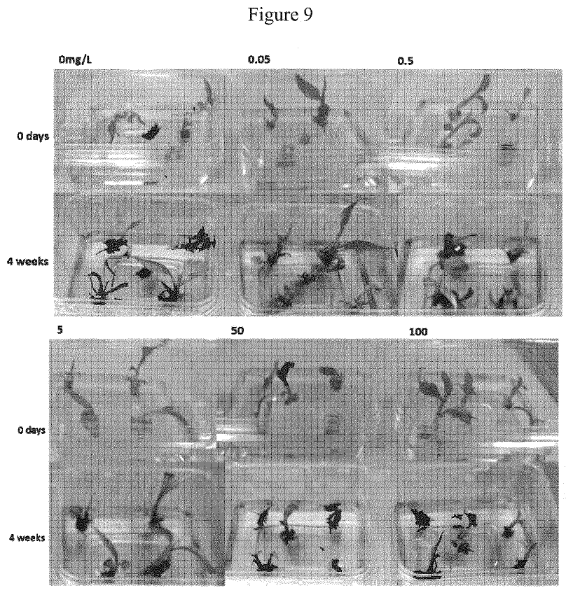

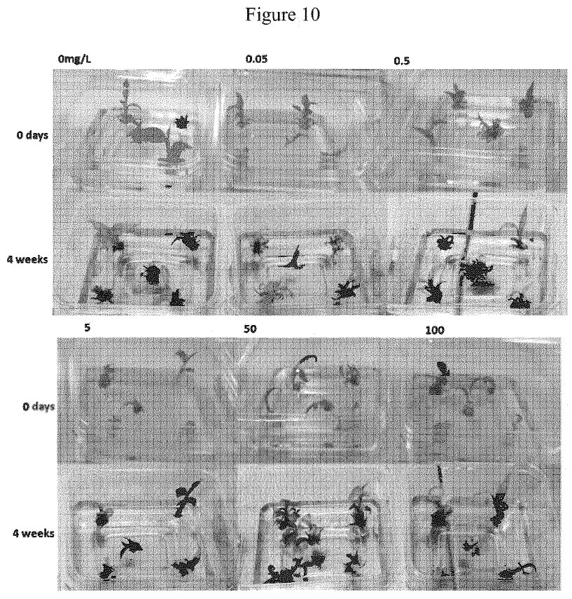

Incorporation of CSF in medium—results shown in FIG. 4 and FIG. 5.
Spraying banana plantlets with CSF—results shown in FIG. 6, FIG. 7 and FIG. 8.
Spraying banana plantlets with CSF plus incorporation of 0.05 mg/L in the medium—results shown in FIG. 9 and FIG. 10.

A range of CSF concentrations (0, 0.05, 0.5, 5, 50, 100 mg/L) were tested in banana rooting shoots. Banana shoots were separated from roots and split to give single shoots that were inoculated into new media to determine the effect of CSF over a month of exposure. To assess the effectiveness of CSF in banana three approaches were tested: (i) incorporation of the toxin in RM medium, (ii) spraying the shoots, and (iii) spraying the shoots plus incorporation of a small amount of CSF (0.05 mg/L) in RM.

Adding CSF to the medium was effective at 0.5 mg/L. The plantlets did not develop and showed a chlorotic phenotype. In the presence of CSF root development was not observed even at lower concentrations, like 0.05 mg/L. These experiments were repeated 3 times and the results were consistent.

Spraying banana shoots seemed to be less effective than incorporating CSF in the medium. 5 mg/L of CSF was the lowest concentration that delayed shoot development. Using Silwett L-77 in the CSF solutions gave rise to a more severe phenotype. Weekly spraying with 0.5 mg/L of CSF was quite effective at delaying banana shoot development.

Combining the two approaches, incorporation of a small amount of CSF, 0.05 mg/L, in the medium plus spraying with CSF, led to an increase in the severity of symptoms. Having a small amount of CSF present in the medium was enough to potentiate the effect of CSF solutions (even when a mock solution was used for spraying (no CSF), a low level of symptoms was seen). In these conditions the banana shoots were quite sick and didn't develop during the period of the experiment.

### Example 2: Identification of Banana ALS Genes

Specific ALS edits that are selectable by chlorsulfuron treatment have been described in potato and tomato (Veillet F, et al. 2019. Int J Mol Sci 20:402). The sequences of these ALS peptides were identified, downloaded, and used to interrogate the banana genome/derived proteome (Droc et al., The banana genome hub. Database, 2013).

### Potato:

stALS1
>tr|M1D7U6|M1D7U6_SOLTU Acetolactate synthase OS=Solanum tuberosum OX=4113 GN=102594064 PE=3 SV-1
SEQ ID NO: 1
stALS2 splice variant 1
>tr|M1AAA1|M1AAA1_SOLTU Acetolactate synthase OS=Solanum tuberosum OX=4113 GN=102595762 PE=3 SV=1
SEQ ID NO:2
stALS2 splice variant 2
>tr|M1AAA0|M1AAA0_SOLTU Acetolactate synthase OS=Solanum tuberosum OX=4113 GN=102595762 PE=3 SV-1
SEQ ID NO:3

### Tomato:

S1ALS1
>tr|A0A3Q7FJD2|A0A3Q7FJD2_SOLLC Acetolactate synthase OS=Solanum 35 lycopersicum OX=4081 GN=101247891 PE=3 SV=1
SEQ ID NO:4
S1ALS2
>tr|A0A3Q7HE60|A0A3Q7HE60_SOLLC Acetolactate synthase OS=Solanum 40 lycopersicum OX=4081 PE=3 SV=1
SEQ ID NO:5
   ALS has also been described in Arabidopsis (Sathasivan et al., 1990):
AtALS1
>sp|P17597|ILVB_ARATH Acetolactate synthase, chloroplastic OS=Arabidopsis thaliana OX=3702 GN=ALS PE=1 SV=1
SEQ ID NO:6

Each of the five potato and tomato amino acid sequences was used as a query in a TBLASTN BLAST search against a custom banana genome database in Geneious Prime software (www.geneious.com). There were only two hits above 30% ID (identity) at amino acid level for each query. These are shown in Table 3 below. Herein Ma06_g18100 is referred to as MaALS1 and Ma10_g11980 is referred to as MaALS2.

Table 3 Closest homologues to known ALS genes in the banana proteome, and the identity expressed as the percentage of residues which are identical between the query and subject peptide sequence.

| | % ID to | % ID to | | % ID of |
|---|---|---|---|---|
| | Ma06_g18100 - | Ma10_g11980 - | | nearest next bit |
| | MaALS1 | MaALS2 | Next closest hit | (>100 bp length) |
| Arabidopsis | 76.4 | 76.9 | Ma01_g14210 | 26.4 |
| ALS1 | | | | |
| Tomato | 79.1 | 78.7 | Ma01_g14210 | 25.9 |
| ALS1 | | | | |
| Tomato | 78.6 | 78.5 | Ma01_g14210 | 26.1 |
| ALS2 | | | | |
| Potato ALS1 | 79.1 | 79.3 | Ma01_g14210 | 25.6 |
| Potato | 80.2 | 79.6 | Ma01_g14210 | 26.6 |
| ALS2a | | | | |
| Potato | 79.1 | 79 | Ma01_g14210 | 25.8 |
| ALS2b | | | | |

The sequences of MaALS1 and ALS2 are shown below.
>ALS1 Ma06_g18100-Ma06_g18100
SEQ ID NO:7
>ALS2 Ma10_g11980-Ma10_g11980
SEQ ID NO:8
>ALS1 Ma06_g18100-Ma06_g18100-CDS translation
SEQ ID NO: 9
>ALS2 Ma10_g11980-Ma10_g11980-CDS translation
SEQ ID NO:10

In summary, the two putative ALS genes in banana have high levels of homology to known ALS genes from Arabidopsis, tomato, and potato. There were no other candidate genes found in the current banana genome “DH-Pahang v2.0” (available from: https://banana-genome-hub.southgreen.fr/organism/1).

### Example 3: Expression Analysis of ALS1/ALS2 in Banana

This example tests the transcriptional expression profile of ALS1 and ALS2 in banana. RNA was extracted from a variety of different banana tissues, as detailed in Table 4 below. RNA libraries were prepared using Illumina Truseq total RNA kit with plant Ribozero and sequencing was performed using HiSeq 300 cycle (2×150 bp) high output. This total RNA-seq generated ˜44 million reads per sample (Cambridge Genomic Services).

TABLE 4
Sample description for banana tissues used to generate RNA samples used in RNAseq dataset.

| | | | | | Biological | ID of Sequencing |
|---|---|---|---|---|---|---|
| Tissue | Description | Plant age | | Variety | Replicates | Data |
| Rolled Leaf | Rolled leaf from soil- | 2-3 | months | Grand Nain Israel | 2x | Rolled_Leaf_1 |
| | grown plant (grown | | | | | Rolled_Leaf_5 |
| | in West-1 CER) | | | | | |
| Top Expanded | Youngest expanded | 2-3 | months | Grand Nain Israel | 2x | Top_Leaf_1 |
| Leaf | leaf from soil-grown | | | | | Top_Leaf_5 |
| | plant (grown in West- | | | | | |
| | 1 CER) | | | | | |
| Root | Roots from a soil- | 5 | months | Grand Nain Israel | 2x | Root_1 |

| | | | | | |
|---|---|---|---|---|---|
| | grown plant (grown | | | | Root_2 |
| | in West-1 CER) | | | | |
| Peel - Green | Peel from a green | N/A | Unknown | 2x | Green_Peel_1A |
| | banana (purchased at | | (potentially Grand | | Green_Peel_2A |
| | a supermarket) | | Nain) | | |
| Peel - Yellow | Peel from a yellow | N/A | Unknown | 2x | Yellow_Peel_1A |
| | banana (purchased at | | (potentially Grand | | Yellow_Peel_2A |
| | a supermarket) | | Nain) | | |
| Flesh - Green | Flesh from a green | N/A | Unknown | 2x | Green_Flesh_1 |
| | banana (purchased at | | (potentially Grand | | Green_Flesh_2 |
| | a supermarket) | | Nain) | | |
| Flesh - Yellow | Flesh from a yellow | N/A | Unknown | 2x | Yellow_Flesh_1 |
| | banana (purchased at | | (potentially Grand | | Yellow_Flesh_2 |
| | a supermarket) | | Nain) | | |

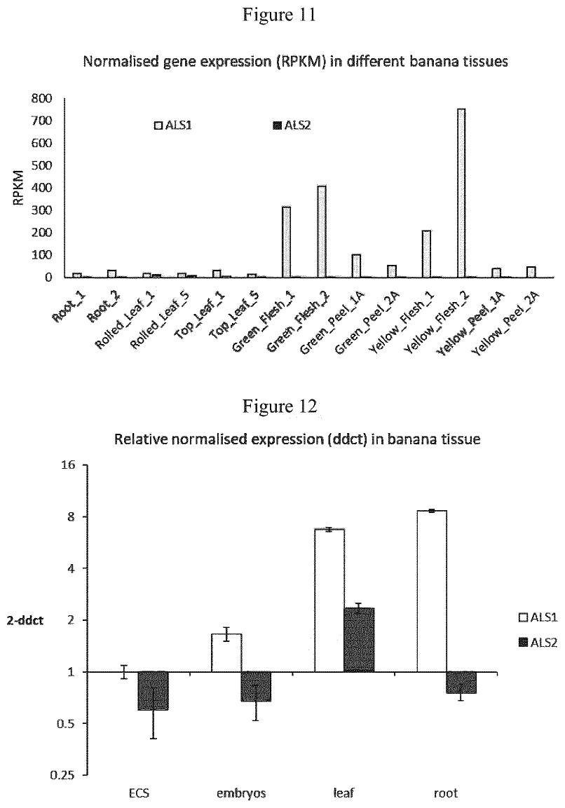

The resultant RNA sequencing reads were trimmed using trim_galore, and fastQC, and mapped to the Musa acuminata DH Pahang v2 (released January 2016) from the banana genome hub (Droc 2013) using HISAT2 (Kim, et al., 2019, Nature Biotechnology, 37(8), 907-915). Counts and TPM calculation were made using Kallisto (Bray, 2016, Nature Biotechnology, 34(5), RPKM and TMM were calculated using R and EdgeR scripts (McCarthy, et al., 2012. Nucleic Acids Research, 40(10), 4288-4297, Robinson et al., 2009, Bioinformatics, 26(1), 139-140). These data were used to prepare FIG. 11, which shows the RPKM values of banana ALS1 and ALS2 across all tissue types sampled.

To study the transcriptional profile of ALS1 and ALS2 in early developmental stages, and to validate the RNAseq data from root and leaf samples, RT-qPCR was performed.

All tissue samples were from banana Grand Nain (GN_236), we used embryonic cell suspensions derived from male flowers (ECS), derived embryos regenerated from these cells, and leaf and root material from plants germinated from these embryos.

RNA was extracted using the Direct-zol kit, following the manufacture instructions, samples were DNase treated in column. Concentration and quality were assessed, before equal amounts of RNA were used to generate cDNA using Turbo-DNase (Ambion) and Superscript IV VILO (Thermofisher).

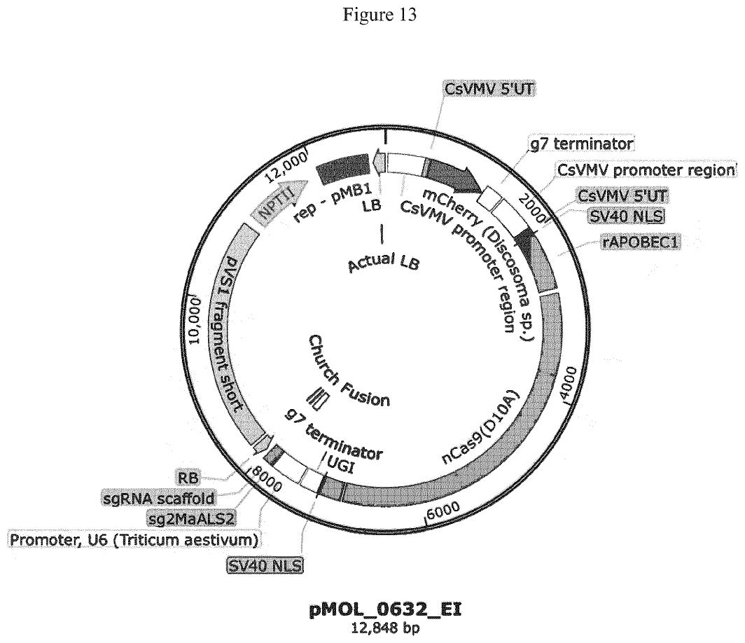

To assess the level of transcript in each sample, we used PowerUp™ SYBR™ Green Master Mix (Applied Biosystems) in RT-qPCR reactions, in a Roche LightCycler 96 system. Data were analysed using the ΔΔCt method in excel and are presented in Table 5 and FIG. 12. Expression of ALS1 and ALS2 were normalised to primer efficiency, and using RPS2 expression, and all data are shown relative to ALS1 expression in ECS.

Amplicons were generated using oligonucleotide primers:

ALSIF
(SEQ ID NO: 11)
GGTCTTCGCTGCCTCTGGAT,
ALSIR (primer no. 2365)
(SEQ ID NO: 12)
CCACCACCAACATAGAGGACT,
ALS2F (primer no. 2366)
(SEQ ID NO: 13)
ATCTGTTCGCCTACCCTGGA,
ALS2R
(SEQ ID NO: 14)
GATGGATCTGGTGACCTCGAC,
RPS2F
(SEQ ID NO: 15)
TAGGGATTCCGACGATTTGTTT
and
RPS2R
(SEQ ID NO: 16)
TAGCGTCATCATTGGCTGGGA.

TABLE 5
Calculated relative normalised expression data

| | | | | | sEofdiff (standard error | | | |
|---|---|---|---|---|---|---|---|---|
| | 2power-ddct | | | | of difference) | | | |
| | ECS | embryos | leaf | root | ECS | embryos | leaf | root |
| ALS1 | 1 | 1.7 | 6.7 | 8.6 | 0.1 | 0.15 | 0.2 | 0.1 |
| ALS2 | 0.6 | 0.7 | 2.3 | 0.8 | 0.2 | 0.16 | 0.16 | 0.08 |

The data presented in FIG. 11 and FIG. 12 show that ALS1 is expressed more highly than ALS2 in all tissues tested. ALS1 is most highly expressed in the fruit stage of the banana, whereas ALS2 peak expression is in banana leaf tissue. Expression of both ALS1 and ALS2 is low in ECS and embryos relative to leaf, root and fruit.

### Example 4: Selection of a Mutation to Introduce in ALS1 and ALS2

The ALS enzyme is the target of several classes of herbicides: sulfonylurea (SU), imidazolinone (IMI), pyrimidinyl-thiobenzoate, sulfonyl-aminocarbonyl-triazolinone and triazolopyrimidine. Hundreds of weed species are known to have naturally developed resistance these compounds. There are 26 resistant amino acid substitutions at eight positions of the ALS gene in 50 weed species (Yu & Powles, 2014a, Pest Management Science, 70(9)). The most frequently occurring mutation in ALS are at Pro-197, Trp-574-Leu and Ala-121; these mutations generally confer resistance to specific herbicides. Table 1 above shows known ALS mutations that have been described, and herbicide binding for such amino acid sites in Arabidopsis.

Relevant for this work, the most common mutation found across all weed species is Pro-197. This mutation confers resistance to sulfonylurea compounds such as chlorsulfuron.

Arabidopsis has only one ALS gene, and the amino acid change Pro-197-Ser confers strong resistance to the sulfonylurea compound chlorsulfuron (Haughn, et al., 1988, MOG Molecular & General Genetics, 211(2), 266-271). Mutations in ALS genes have been described and confer resistance to sulfonylurea compounds in several crop species, including tomato (Jung et al., 2004, Biochemical Journal, 383(1), 53-61 and Veillet F, et al. 2019. Int J Mol Sci 20:402), potato (Barrel et al. 2017. BMC Biotechnology 17:49 and Veillet F, et al. 2019), maize (Li et al., 1992. Plant Physiol 100:662-668), tobacco (Jung et al., 2004) and rice (Wakasa et al., 2012, Plant Cell Reports, 31(11), 2075-2084).

Banana has two ALS homologues, as described earlier. To identify the particular amino acid residue that corresponds to the Pro-197 residue from Arabidopsis we compared alignments of the peptide sequences from several species of plants.

The Pro197 residue is highlighted below in bold and underlined. There is a very high degree of conservation of the amino acid sequence in this region.

Note that in banana ALS1, the proline mutation required is Pro187Ser, and in ALS2 it is Pro181Ser.

| | |
|---|---|
| ALS2 Ma10_g11980- | SEQ ID NO: 10 |
| ALS1 Ma06_g18100- | SEQ ID NO: 9 |
| sp\|P17597\|ILVB_ARATH- | SEQ ID NO: 6 |
| tr\|A0A3Q7HE60\|A0A3Q7HS60_SOLLC- | SEQ ID NO: 5 |
| tr\|M1AAA1\|M1AAA1_SOLTU- | SEQ ID NO: 2 |
| tr\|M1AAA0\|M1AAA0_SOLTU- | SEQ ID NO: 3 |
| tr\|M1D706\|M1D7U6_SOLTU- | SEQ ID NO: 1 |
| tr\|A0A3Q7FJD2\|A0A3Q7FJD2_SOLLC- | SEQ ID NO: 4 |
| ALS2 Ma10_g11980 | -MASST------PGAAIAPSNPSLWPFSAASRLLVPFPKPLLSLSPSHRNVRPISASADR 53 |
| ALS1 Ma06_g18100 | -MASSAAAAVAAAGAVITPSKSSHPPFSAAYRLPFPLPKPLHSLSTRHPEVRPISASADR 59 |
| sp\|P17597\|ILVB_ARATH | -MAAATTTTTTSSSISFS---TKPSP--SSSKSPLPISRFSLPFSLNPNKSSSSSRRRGI 54 |
| tr\|A0A3Q7HE60\|A0A3Q7HS60_SOLLC | -MAAAS------PSPCFS---KTLPP--SSSKSSTLLPKSTFTFHNHPKKASPLHLTHTQ 48 |
| tr\|M1AAA1\|M1AAA1_SOLTU | -MAAAS------PSPCFS---KNLPP--SSSKSSTLLPKSTFTFHNHPKNTSPLHLTHTQ 48 |
| tr\|M1AAA0\|M1AAA0_SOLTU | -MAAAS------PSPCES---KNLPP--SSSKSSTLLPKSTFTFHNHPKNTSPLHLTHTQ 48 |
| tr\|M1D706\|M1D7U6_SOLTU | MAAAAS------PSPCFS---KTLPP--SSSKSSTILPRSTFPFHNHPQKASPLHLTHTH 49 |
| tr\|A0A3Q7FJD2\|A0A3Q7FJD2_SOLLC | MAAAAS------PSPCES---KTLPP--SSSKSSTILPRSTFPFHNHPQKASPLHLIHAQ 49 |
| ALS2 Ma10_g11980 | HQTPSAAAS-------------TAATTAVP--LLRNFAPDEPRKGSDILVEALEREGVTD 98 |
| ALS1 Ma06_g18100 | RQLPSAAAP-------------TADATTAP--LLRNFAPDEPRKGADILVEALEREGVTD 104 |
| sp\|P17597\|ILVB_ARATH | KSSSPSSISAVLNTTTNVTTTPSPTKPTKPETFISRFAPDQPRKGADILVEALERQGVET 114 |
| tr\|A0A3Q7HE60\|A0A3Q7HS60_SOLLC | HHSR-FTVSNVILSTT------THDDVSEPETFVSRFAPDEPRKGCDVLVEALEREGVKD 101 |
| tr\|M1AAA1\|M1AAA1_SOLTU | HHSR-FTVSNVILSTT------THNDVSEPEIFVSRFAPDEPRKGCDVLVEALEREGVKD 101 |
| tr\|M1AAA0\|M1AAA0_SOLTU | HHSR-FTVSNVILSTT------THNDVSEPEIFVSRFAPDEPRKGCDVLVEALEREGVKD 101 |
| tr\|M1D706\|M1D7U6_SOLTU | HHRRGFAVSNVVISTT------THNDVSEPETFVSRFAPDEPRKGCDVLVEALEREGVTD 103 |
| tr\|A0A3Q7FJD2\|A0A3Q7FJD2_SOLLC | HNRRGFAVANVVISTT------THNDVSEPETFVSRFAPDEPRKGCDVLVEALEREGVTD 103 |
| ALS2 Ma10_g11980 | LFAYPGGASMEIHQALTRSPSITNHLLRHEQGEIFAASGYARSTGRPGVCIATSGPGATN 158 |
| ALS1 Ma06_g18100 | LFAYPGGASMEIHQALTRSPSITNHLLRHEQGEVFAASGYARSTGRPGVCIATSGPGATN 164 |
| sp\|P17597\|ILVB_ARATH | VFAYPGGASMEIHQALTRSSSIRNVLPRHEQGGVFAAEGYARSSGKPGICIATSGPGATN 174 |
| tr\|A0A3Q7HE60\|A0A3Q7HS60_SOLLC | VFAYPGGASMEIHQALTRSNIIRNVLPRHEQGGVFAAEGYARATGFPGVCIATSGPGATN 161 |
| tr\|M1AAA1\|M1AAA1_SOLTU | VFAYPGGASMEIHQALTRSNIIRNVLPRHEQGGVFAAEGYARATGFPGVCIATSGPGATN 161 |
| tr\|M1AAA0\|M1AAA0_SOLTU | VFAYPGGASMEIHQALTRSNIIRNVLPRHEQGGVFAAEGYARATGFPGVCIATSGPGATN 161 |
| tr\|M1D706\|M1D7U6_SOLTU | VFAYPGGASMETHQALTRSNIIRNVLPRHEQGGVFAAEGYARATGFPGVCTATSGPGATN 163 |
| tr\|A0A3Q7FJD2\|A0A3Q7FJD2_SOLLC | VFAYPGGASMEIHQALTRSNIIRNVLPRHEQGGVFAAEGYARATGFPGVCIATSGPGATN 163 |
| ALS2 Ma10_g11980 | LVSGFADALLDSVPLVAITGQVPRRMIGTDAFQETPIVEVTRSITKHNYLVLNVDDIPRI 218 |
| ALS1 Ma06_g18100 | LVSGLADALLDSVPLVAITGQVPRRMIGTDAFQETPIVEVTRSITKHNYLVINVDDIPRI 224 |
| sp\|P17597\|ILVB_ARATH | DVSGLADALLDSVPLVAITGQVPRRMIGTDAFQETPIVEVTRSITKHNYLVMDVEDIPRI 234 |
| tr\|A0A3Q7HE60\|A0A3Q7HS60_SOLLC | LVSGLADALLDSIPIVAITGQVPRRMIGTDAFQETPIVEVTRSITKHNYLVMDVEDIPRV 221 |
| tr\|M1AAA1\|M1AAA1_SOLTU | LVSGLADALLDSIPIVAITGQVPARMIGTDAFQETPIVEVTRSITKHNYLVMDVEDIPRV 221 |
| tr\|M1AAA0\|M1AAA0_SOLTU | LVSGLADALLDSIPIVAITGQVPRRMIGTDAFQETPIVEVTRSITKHNYLVMDVEDIPRV 221 |
| tr\|M1D706\|M1D7U6_SOLTU | LVSGLADALLDSIPIVAITGQVPRRMIGTDAFQETFIVEVTRSITKHNYLVMDVEDIPRV 223 |
| tr\|A0A3Q7FJD2\|A0A3Q7FJD2_SOLLC | LVSGLADALLDSIPIVAITGQVPRRMIGTDAFQETFIVEVTRSITKHNYLVMDVEDIPRV 223 |
| ALS2 Ma10_g11980 | IKEAFFLASTGRPGPVLVDIPKDIQQQLAIPVWDPPLRLPGYISRLPKPPSRCLLDQIIR 278 |
| ALS1 Ma06_g18100 | IKEAFFVATTGRPGPVLVDIPKDIQQQLAIPVWDPPLRLPGYISRLPREPARPLLDQILR 284 |
| sp\|P17597\|ILVB_ARATH | IEEAFFLATSGRPGPVLVDVPKDIQQQLAIPNWEQAMRLPGYMSRMPKPPEDSHLEQIVR 294 |
| tr\|A0A3Q7HE60\|A0A3Q7HS60_SOLLC | VREAFFLAKSGRPGPVLIDVPKDIQQQLVIPNWDQPMRLPGYMSRLPKLPNEMLLEQIVR 281 |
| tr\|M1AAA1\|M1AAA1_SOLTU | VREAFFLAKSGRPGPVLIDVPKDIQQQLVIPNWDQPMREPGYISRLPKLPNEMLLEQIVR 281 |
| tr\|M1AAA0\|M1AAA0_SOLTU | VREAFFLAKSGRPGPVLIDVPKDIQQQLVIPNWDQPMREPGYISRLPKLPNEMLLEQIVR 281 |
| tr\|M1D706\|M1D7U6_SOLTU | VREAFFLAKSGRPGPVLIDVPKDIQQQLVIPNWDQPMRLPGYMSRLPKLPNEMLLEQIIR 283 |
| tr\|A0A3Q7FJD2\|A0A3Q7FJD2_SOLLC | VREAFFLAKSGRPGPVLIDVPKDIQQQLVIPNWDQPMRLPGYMSRLPKLPNEMLLEQIVR 283 |
| ALS2 Ma10_g11980 | LVSESERPVLYVGGGCINSSEELERFADLTGIPIASTLMGLGVYPTDAELSLKMLGMHGT 338 |
| ALS1 Ma06_g18100 | LVSESRRPVLYVGGGCINSSEELARFADLTGIPIASTLMGLGAYPTDAELSLKMLGMHGT 344 |
| sp\|P17597\|ILVB_ARATH | LISESKKPVLYVGGGCLNSSDELGREVELIGIPVASTLMGLGSYPCDDELSLHMLGMHGT 354 |
| tr\|A0A3Q7HE60\|A0A3Q7HS60_SOLLC | LISESKKPVLYVGGGCSQSSEELRRFVELIGIPVASTLMGLGAFPSGDELSLQMLGMHGT 341 |
| tr\|M1AAA1\|M1AAA1_SOLTU | LISESKKPVLYVGGGCTQSSEELRREVELTGIPVASTLMGLGTFPCGDELSLQMLGMHGT 341 |
| tr\|M1AAA0\|M1AAA0_SOLTU | LISESKKPVLYVGGGCTQSSEELRREVELTGIPVASTLMGLGTFPCGDELSLQMLGMHGT 341 |
| tr\|M1D706\|M1D7U6_SOLTU | LISESKKPVLYVGGGCLQSSEELRRFVELTGIPVASTLMGLGAFPTGDELSLQMLGMHGT 343 |
| tr\|A0A3Q7FJD2\|A0A3Q7FJD2_SOLLC | LISESKKPVLYVGGGCSQSSEELRRFVELTGIPVASTIMGLGAFPTGDELSLQMLGMHGT 343 |
| ALS2 Ma10_g11980 | VYANYSVDKADLLLALGVRFDDRVTGKLEAFASRAKIVHIDIDPAEIGKNKQPHVSLCAD 398 |
| ALS1 Ma06_g18100 | VYANYAIDKADLLLAFGVRFDDRVTGKIEAFASRAKIVHIDIDPAEIGKNKQPHYSICAD 404 |
| sp\|P17597\|ILVB_ARATH | VYANYAVERSDLLLAFGVRFDDRVTGKLEAFASRAKIVHIDIDSAEIGKNKTPHVSVCGD 414 |
| tr\|A0A3Q7HE60\|A0A3Q7HS60_SOLLC | VYANYAVDSSDALLAFGVRFDDRVTGKLEAFASRAKIVHIDIDSAEIGKNKQPHVSICAD 401 |
| tr\|M1AAA1\|M1AAA1_SOLTU | VYANYAVDSSDLLLAFGVRFDDRVIGKLEAFASRAKIVHIDIDSAEIGKNKQPHVSICAD 401 |
| tr\|M1AAA0\|M1AAA0_SOLTU | VYANYAVDSSDLLLAFGVRFDDRVIGKLEAFASRAKIVHIDIDSAEIGKNKQPHVSICAD 401 |
| tr\|M1D706\|M1D7U6_SOLTU | VYANYAVDGSDLLLAFGVRFDDRVIGKLEAFASRAKIVHIDIDSAEIGKNKQPHVSICAD 403 |
| tr\|A0A3Q7FJD2\|A0A3Q7FJD2_SOLLC | VYANYAVDSSDALLAFGVRFDDRVTGKLEAFASRAKIVHIDIDSAEIGKNKQPHVSICAD 403 |
| ALS2 Ma10_g11980 | VRIALQGMNALMEESGIMQKFDFSTWRKELDQLKKAYPLSYKTFGDLIPPQYAIQVLDEL 458 |
| ALS1 Ma06_g18100 | VKLALQGMNALMEETEIYRKEDFSTWREELDKQKKIYPLNYKTFGDQIPPQYAIQVLDEL 464 |
| sp\|P17597\|ILVB_ARATH | VKLALQGMNKVLENRAEELKLDFGVWRNELNVQKQKFPLSFKTFGEAIPPQYAIKVLDEL 474 |
| tr\|A0A3Q7HE60\|A0A3Q7HS60_SOLLC | VKLALQGMNKVLENRAEELKLDFGVWRNELNVQKQKFPLSFKTFGEAIPPQYAIKVLDEL 461 |
| tr\|M1AAA1\|M1AAA1_SOLTU | IKLALQGINSIFESKKGKLKLDESAWRQELTEQKVKYPLNFKTFGEAIPPQYAIQVLDEL 461 |
| tr\|M1AAA0\|M1AAA0_SOLTU | IKLALQGINSILEGKEGKLKLDFSAWRQELTEQKVKYPINYKTEGEAIPPQYAIQVLDEL 461 |
| tr\|M1D706\|M1D7U6_SOLTU | IKLALQGINSILEGKEGKLKLDESAWRQELTEQKVKYPLSFKTEGEAIPPQYAIQVLDEL 463 |
| tr\|A0A3Q7FJD2\|A0A3Q7FJD2_SOLLC | IKLALQGLNSILEGKEGKMKLDFSAWRQELTEQKMKYPLNFKTFGDAIPPQYAIQVLDEL 463 |
| ALS2 Ma10_g11980 | TNGEAIISTGVGQHQMWAAQYYSYKRARQWLTSGGLGAMGFGLPAAAGAAVGNPGVTVVD 518 |
| ALS1 Ma06_g18100 | INGEAIITTGVGQHQMWAAQYYSYKRARQWLTSAGLGAMGFGLPAAAGAAVGNPGVTVVD 524 |
| sp\|P17597\|ILVB_ARATH | TDGKAIISTGVGQHQMWAAQFYNYKKPRQWLSSGGLGAMGFGLPAAIGASVANPDAIVVD 534 |
| tr\|A0A3Q7HE60\|A0A3Q7HS60_SOLLC | TNGNAIISTGVGQHQMWAAQHYKYKKPRQWLTSGGLGAMGFGLPAAIGAAVGRPGEIVVD 521 |
| tr\|M1AAA1\|M1AAA1_SOLTU | TNGNAIISTGVGQHQMWAAQYYKYKKPRQWLISGGLGAMGFGLPAAIGAAVGRPGEIVVD 521 |
| tr\|M1AAA0\|M1AAA0_SOLTU | TNGNAIISTGVGQHQMWAAQYYKYKKPRQWLISGGLGAMGFGLPAAIGAAVGRPGEIVVD 521 |
| tr\|M1D706\|M1D7U6_SOLTU | TNGNAIISTGVGQHQMWAAQYYKYKKPRQWLTSGGLGAMGFGLPAAIGAAVGRPGEIVVD 523 |
| tr\|A0A3Q7FJD2\|A0A3Q7FJD2_SOLLC | TNGNAIISTGVGQHQMWAAQYYKYKKPRQWLTSGGLGAMGFGLPAAIGAAVGRPGEIVVD 523 |
| ALS2 Ma10_g11980 | IDGDGSFQMNAQELAMIRIENLPVKMMVLNNQHLGMVVQWEDRFYRSNRGHTYLGNPANE 578 |
| ALS1 Ma06_g18100 | IDGDGSFQMNAQELAMIRIENLPVKIMVLNNQHLGMVVQWEDRFYHRNRAKTYIGNPANE 584 |
| sp\|P17597\|ILVB_ARATH | IDGDGSFIMNVQELATIRVENLPVKVLLLNNQHLGMVMQWEDRFYKANRAHTFLGDPAQE 594 |
| tr\|A0A3Q7HE60\|A0A3Q7HS60_SOLLC | IDGDGSFIMNVQELATIKVENLPVKIMLLNNQHLGMVVQWEDRFYKANRAHTYLGNPANE 581 |
| tr\|M1AAA1\|M1AAA1_SOLTU | IDGDGSFIMNVQELATIKVENLPVKIMLLNNQHLGMVVQWEDREYKANRAHTYLGNPANE 581 |
| tr\|M1AAA0\|M1AAA0_SOLTU | ID---------------------------------------------------------- 523 |
| tr\|M1D706\|M1D7U6_SOLTU | IDGDGSFIMNVQELATIKVENLPVKIMLLNNQHLGMVVQWEDRFYKANRAHTYLGDPANE 583 |
| tr\|A0A3Q7FJD2\|A0A3Q7FJD2_SOLLC | IDGDGSFIMNYCELATIKVENLPVKIMLLNNQHLGMVVQWEDRFYKANRAHTYLGDPSNE 583 |
| ALS2 Ma10_g11980 | SEIFPDELKITEAYGIPAARVTKKSEVREAIRKMLKTPGPYLLDVIVPHEEHVLPMIPSG 638 |
| ALS1 Ma06_g18100 | TEVFPDFLKISEGYGIPAARVTKKSEVREAIRKMLETSGPYLLDVIVPHEEHVLPMIPSG 644 |
| sp\|P17597\|ILVB_ARATH | DEIFPNMLLFAAACGIPAARVTKKADLREAIQTMLDTPGPYLLDVICPHQEHVLPMIPSG 654 |
| tr\|A0A3Q7HE60\|A0A3Q7HS60_SOLLC | EEIFPNMLKFAEACGVPAARVSERDDLRAAIQKMLDTPGPYLLDVIVPHQEHVLPMIPSG 641 |
| tr\|M1AAA1\|M1AAA1_SOLTU | EEIFPNMLKFAEACGVPAARVSERDDLRAAIQKMLDTPGFYLLDVIVPHQEHVLPMIPSG 641 |
| tr\|M1AAA0\|M1AAA0_SOLTU | -----------EACGVPAARVSHRDDLRAAIQKMLDTPGPYLLDVIVPHQEHVLPMIPSG 572 |
| tr\|M1D706\|M1D7U6_SOLTU | EEIFPNMLKFAEACGVPAARVSERDDLRAAIQKMLDIPGPYLLDVIVPHQEHVLPMIPSG 643 |
| tr\|A0A3Q7FJD2\|A0A3Q7FJD2_SOLLC | EEIFPNMLKFAEACGVPAARVSHRDDLRAAIQKMLDTPGPYLLDVIVPHQEHVLPMIPSG 643 |
| ALS2 Ma10_g11980 | GAFKDMILDGDGRTPPH------------------------------------------- 655 |
| ALS1 Ma06_g18100 | GAFKDMILDGDGRTPY-------------------------------------------- 660 |
| sp\|P17597\|ILVB_ARATH | GTFNDVITEGDGRIKY-------------------------------------------- 670 |
| tr\|A0A3Q7HE60\|A0A3Q7HS60_SOLLC | GAFKDVITEGDGRRSY-------------------------------------------- 657 |
| tr\|M1AAA1\|M1AAA1_SOLTU | GAFKDVITEGDGRRSY-------------------------------------------- 657 |
| tr\|M1AAA0\|M1AAA0_SOLTU | GAFKDVITEGDGRRSY-------------------------------------------- 588 |
| tr\|M1D706\|M1D7U6_SOLTU | GAFKDVITEGDGRRSY-------------------------------------------- 659 |
| tr\|A0A3Q7FJD2\|A0A3Q7FJD2_SOLLC | GAFKDVITEGDGRCSY-------------------------------------------- 659 |

### Example 5: Introduction of Mutations in the ALS1 or ALS2 Genes

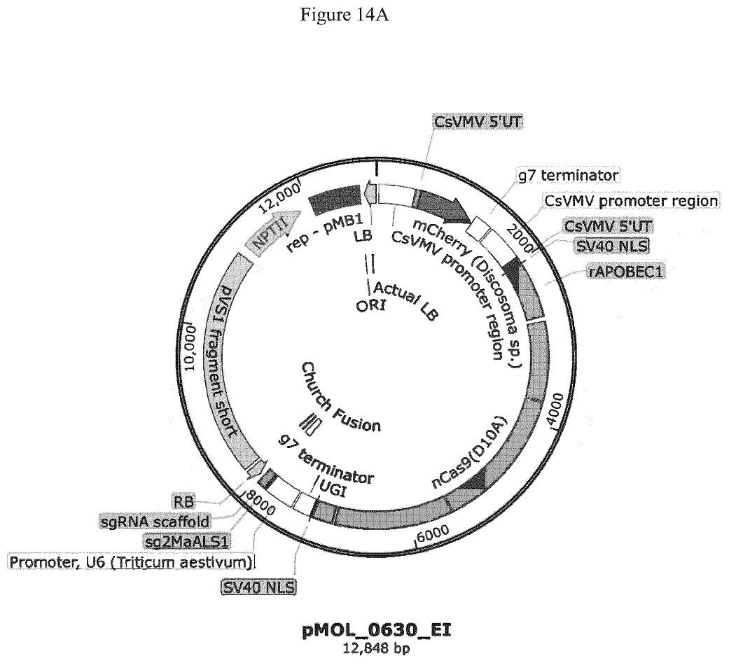

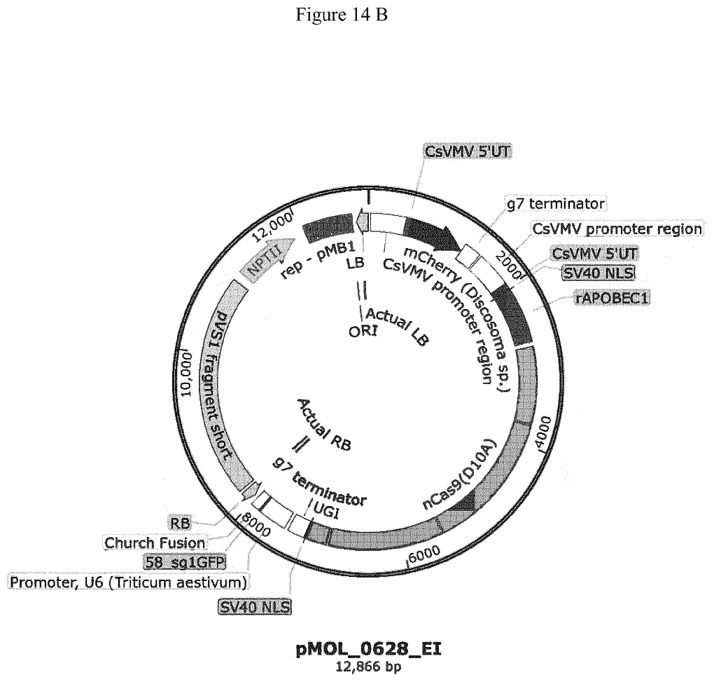

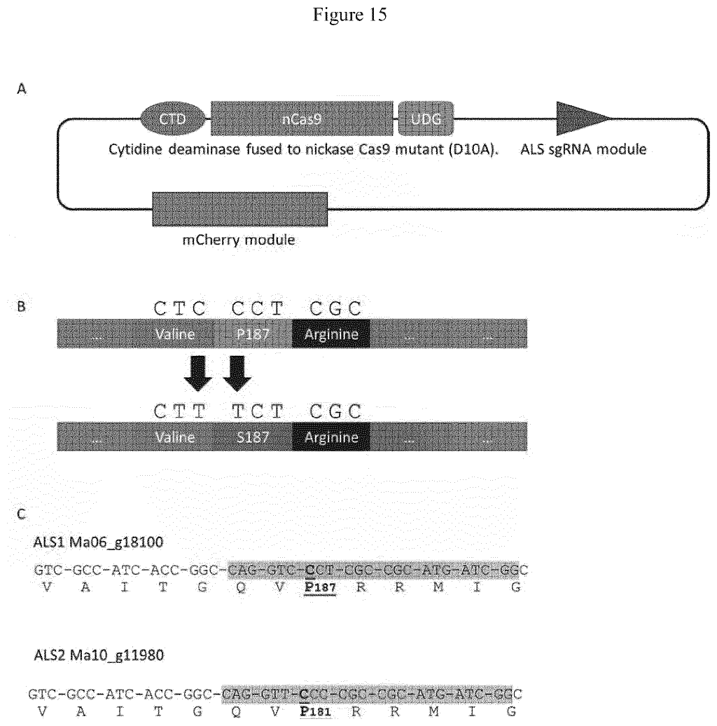

Maps of the plasmids used in this Example are shown in FIGS. 13 and 14.

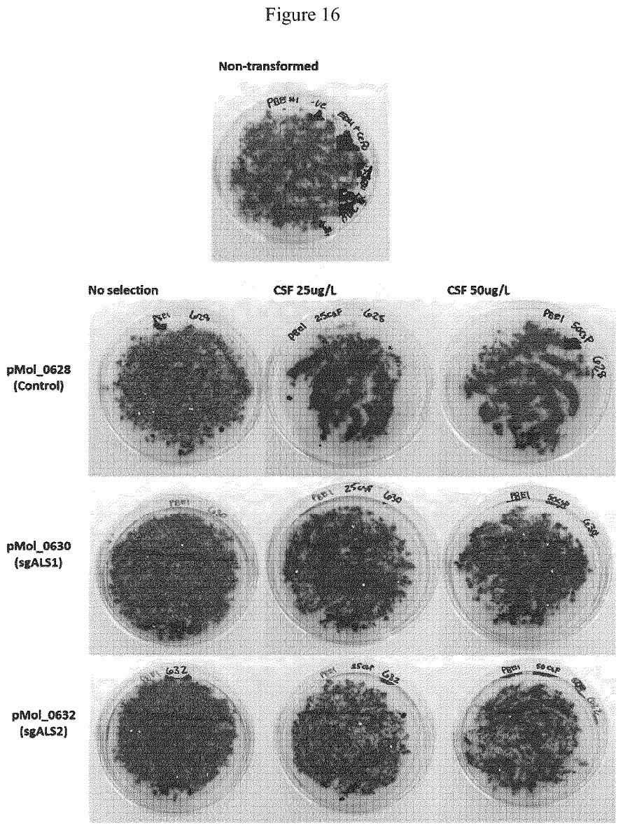

To create the specific point mutations in banana ALS1 and ALS2 genes that would confer resistance to CSF (ALS1 Pro187Ser, ALS2 Pro181Ser), we designed a precise base editor (PBE) expression construct. We based our construct on one used to generate wheat CSF resistance (Zong et al. Nat Biotechnol. 2017, 35(5):438-440) which used a rAPOBEC (Komor et al., 2016, Nature, 533) cytidine deaminase fused to a D10A mutant form of Cas9 (nCAs9) and an uracil deglycosylase, refer to FIGS. 13-15. This fusion protein can use sgRNA-guided Cas9 to deliver the cytidine deaminase and uracil DNA glycosylase (UDG) to a precise editing ‘window’ of DNA sequence on the same strand as the sgRNA, where it can modify a one or more cytidine nucleotide bases (C) into thymine nucleotide (T) bases, e.g., ATCGC would become ATTGT if both C bases were edited. The ‘nickase’ nCas9 (D10A) modification modifies the catalytic activity so that only one strand of the DNA duplex is cleaved when nCas9 binds to the sgRNA loci. This fusion is shown in both nucleotide and peptide fasta sequences below.
>rAPOBEC-nCas9-PBE-UDG rice codon optimised and Golden Gate domesticated NUCLEOTIDE
SEQ ID NO:17
>rAPOBEC-nCas9-PBE-UDG rice codon optimised PEPTIDE
SEQ ID NO:18

To guide the PBE fusion to edit the desired base in ALS1 and ALS2 of banana, guide RNA sequences were designed using Geneious Prime 2019.

The specific guide sequences used were:

ALS1 sgRNA
(SEQ ID NO: 19)
CAGGTCCCTCGCCGCATGAT
and
(SEQ ID NO: 20)
ALS2 sgRNA
CAGGTTCCCCGCCGCATGAT

To demonstrate that these sgRNAs could facilitate the desired edit in the banana ALS1 and ALS2 gene, we performed a protoplast editing experiment. Here, we generated protoplasts from banana ECS cells, and transfected them (protocol below) with the rAPOBEC PBE base editor construct containing either the ALS1 or ALS2 guide, or a control with a non-targeting sgRNA, designated pMOL0630, pMOL0632 and pMOL0628, respectively. These constructs also contained an mcherry fluorescent gene marker cassette. Three days after transfection the protoplasts were sorted using a FACS Melody. A minimum of 2,000 protoplasts were sorted for each plasmid, and for the negative control. A protoplast count of 20,000 events was made to measure the transfection efficiency of each plasmid; the efficiency of every plasmid was >1% and >3% for most plasmids. After sorting, protoplasts were stored at −80° C. prior to DNA extraction.

TABLE 6

| PLASMID | DESCRIPTION |
|---|---|
| PMOL_0628 | - CsVMV::mCherry::tg7 - pCsVMVanCas9PBE1::tg7 - |
| | Dummy - pTaU6::sg1GFP_58 |
| PMOL_0630 | - CsVMV::mCherry::tg7 - pCsVMV::nCas9PBE1::tg7 - |
| | taU6::MaALS1sg2_2021 |
| PMOL_0632 | - CsVMV::mCherry::tg7 - pCsVMV::nCas9PBE1::tg7 - |
| | taU6::MaALS2sg2_2023 |

The level of editing in the ALS1 and ALS2 loci was assessed using amplicon sequencing and next generation sequencing. DNA was purified from the protoplasts using Qiagen DNeasy plant genomic DNA extraction kit and used as template in amplification reactions using Q5 master mix polymerase (NEB) using PCR primers for ALS1 and ALS2.

TABLE 7

| | Temp. ° C. | Time (s) |
|---|---|---|
| PCR steps | (ALS1/ALS2) | (ALS1/ALS2) |
| Initial denaturation | 98 | 60 |
| Denaturation | 98 | 10 |
| Annealing | 67 | 30 |
| Extension | 72 | 75 |
| Final extension | 72 | 300 |

Next, nested PCR was performed on diluted amplicons from the first PCR reaction using primers for ALS1 and ALS2.

TABLE 8

| | Temp. ° C. | Time (s) | Temp. ° C. | Time (s) |
|---|---|---|---|---|
| PCR steps | (ALS1) | (ALS1) | (ALS2) | (ALS2) |
| Initial denaturation | 98 | 60 | 98 | 60 |
| Denaturation | 98 | 10 | 98 | 10 |
| Annealing | 68 | 30 | 67 | 30 |
| Extension | 72 | 30 | 72 | 30 |
| Final extension | 72 | 300 | 72 | 300 |

Amplicons were sent to the company Genewiz for sequencing using Miseq technology. We used Geneious to assess the level of editing in each sample, summarised in Table 9.

TABLE 9
Editing detection in ALS1 and ALS2 loci using Ampseq of protoplast populations.

| | Gene | Construct used | | |
|---|---|---|---|---|
| Sample | target/amplicon | (pMOL) | Description | Summary of AmpSeg |
| 1 | ALS1 | 628 | dummy PBE1 | No editing detected |
| 2 | ALS1 | 630 | ALS1 sgRNA | 2, 3 or 4 C-T base edits detected |
| | | | | in reads. A 1-2 bp edit confers |
| | | | | desired amino acid change, |
| | | | | others confer additional |
| | | | | mutations. Desired 1-2 bp edit is |
| | | | | at 21.7% frequency. |
| 3 | ALS1 | untransfected control | | No editing detected |
| 4 | ALS2 | 628 | dummy PBE1 | No editing detected |
| 5 | ALS2 | 632 | ALS2 sgRNA | 2, 3, and 4 C-T edits detected on |
| | | | | sense strand. 1-2 bp is desired |
| | | | | and is present at 34%. Other |
| | | | | edits are 17% frequency - these |
| | | | | are undesired and cause |
| | | | | additional amino acid changes. |
| 6 | ALS2 | untransfected control | | No editing detected |

The data presented in Table 9 demonstrate that the base editing construct was active and could generate the desired edits required for CSF resistance in banana at a high frequency in transformed populations of banana cells.

### Protoplast Transfection Protocol

Banana protoplasts were isolated from embryogenic cell suspension cultures in a filter sterilised enzymatic digestion mix (2% cellulase RS, 0.05% macerozyme R-10, 0.25% pectolyase Y-23, 0.2% hemicellulose (all products are from Sigma)). Approximately 2.5 mL of settled ECS were inoculated in 5 mL enzymatic solution and incubated overnight at 40-60 rpm in the dark.

Following incubation, tissue and buffer mixture was filtered through a 40 μm filter (Fisherbrand, Fisher Scientific). 1 vol 0.45M mannitol was used to wash the petri dish and then passed through the same filter to dilute the enzyme solution and maximize protoplast collection. Protoplasts were collected and the enzyme solution was removed using centrifugation at 100 g for 4 min. Protoplasts were then resuspended in 10 mL 0.45M mannitol and spun down again at 100 g for 4 min. The protoplasts were resuspended in 10 mL MMG solution (0.4M mannitol, 15 mM MgCl2, 4 mM MES); concentration and cell viability were assessed using hemocytometer and Trypan Blue.

Protoplasts were diluted to a final density 1.5×10\hat{ }6 cells/mL and then they were placed in ice before transfection. 20 μg of plasmids DNA was mixed with 200 μL of protoplasts and after 1 vol of PEG solution (4 g PEG-8000, 4 mL0.5M mannitol, 1 mL 1M CaCL2) was added to the previous mixture.

After a 30 min incubation at room temperature, protoplasts were washed with 10 mL of 0.45M mannitol and collected by centrifugation at 100 g for 4 min. Protoplasts were resuspended in 2 mL W5 (154 mM NaCl, 125 mM CaCl2, 5 mM KCl, 2 mM MES pH5.6) and transferred to 6-well Falcon culture plates and incubated at 25 C in the dark for 3 days.

### Example 6: Banana Embryos Grown from ECS Cells Edited in ALS1/ALS2 can be Selected Using CSF

### Material and Methods

Agrobacterium tumefaciens Preparation

Agrobacterium tumefaciens AGL1 carrying the binary plasmids pMol_0628, pMol_0630, and pMol_0632 were streaked from glycerol in LB solid medium supplemented with rifampicin 50 mg/L, kanamycin 100 mg/L and carbenicillin 50 mg/L and incubated at 28° C. for 2 days. On the second day, the bacteria were inoculated in MG/L medium (discussed in Sarkar et al., Methods Mol Biol. 2013; 966: 187-204) supplemented with the same antibiotics and incubated overnight at 28° C. Next day, the cultures were spun down at 4000 rpm for 10 min to pellet the cells and after they were resuspended in ABIM (Agrobacterium induction media; discussed in Sarkar et al., Methods Mol Biol. 2013; 966: 187-204) to induce virulence and incubated overnight in the dark at room temperature. The OD600 of the bacteria was measured using NanoDrop2000 and normalised to 0.6 in CS liquid media supplemented with acetosyringone (100 μM).

### Banana ECS Agrotransformation

Banana ECS with 2/3 day-old cells was pelleted and resuspended in CS medium. 0.5 mL of the suspension (approximately 0.1 mL of ECS) was split according to the number of transformations into small Eppendorf tubes. The cells were incubated at 45° C. for 5 min and after they were incubated at room temperature for another 5 min. Then, 1 mL of agrobacterium suspension was added to each banana ECS sample. The mixtures were mixed well, spun down at 1000 rpm for 5 min and then incubated for 25 min at room temperature. The banana cells were pooled, and the bacteria suspension was removed. The cells were inoculated in CS plates supplemented with acetosyringone 100 μM and incubated for 3 days at room temperature to promote transformation.

### Banana Embryos Developing and Germination

After 3 days, the banana ECS were collected from the previous media and transferred to CS media supplemented with cefotaxime 300 mg/L. One week later, the banana cells were transferred to EDM plates supplemented with cefo (cefotaxime) 300 mg/L (control), or cefo 300 mg/L and CSF 25 μg/L or cefo 300 mg/L and CSF 50 μg/L. The media was refreshed every 2 weeks until embryos developed. Subsequently, the banana embryos were transferred to maturation media without CSF (EMM—embryo maturation medium) for a month and then, they were transferred to germination medium (GM4) without CSF until shoots developed.

### Fluorescence Microscopy Analysis

Banana embryos were checked for the presence of the T-DNA by checking for the presence of mCherry fluorescence in the embryos.

### Results

### Selection of the ALS Edits

Banana ECS were transformed with binary vectors containing mcherry and the cytidine base editor, plus guides for ALS1 (pMol_0630) and ALS2 (pMol_0632) and mock guides for GFP (pMol_0628, control).

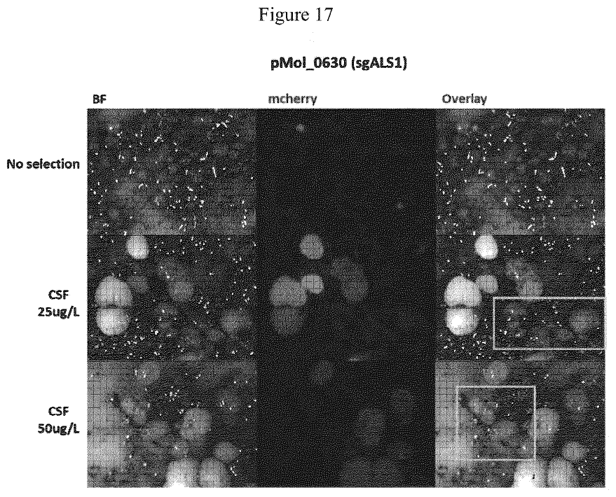

Without selection pressure, embryos were observed in each of the transformation groups. In the presence of CSF no embryos were observed from the control transformation (pMol_0628) (see FIG. 16). It was possible to obtain embryos with CSF selection using the plasmids containing the guides for the ALS1 and ALS2 point mutations. As expected, fewer embryos were observed with higher concentrations of CSF.

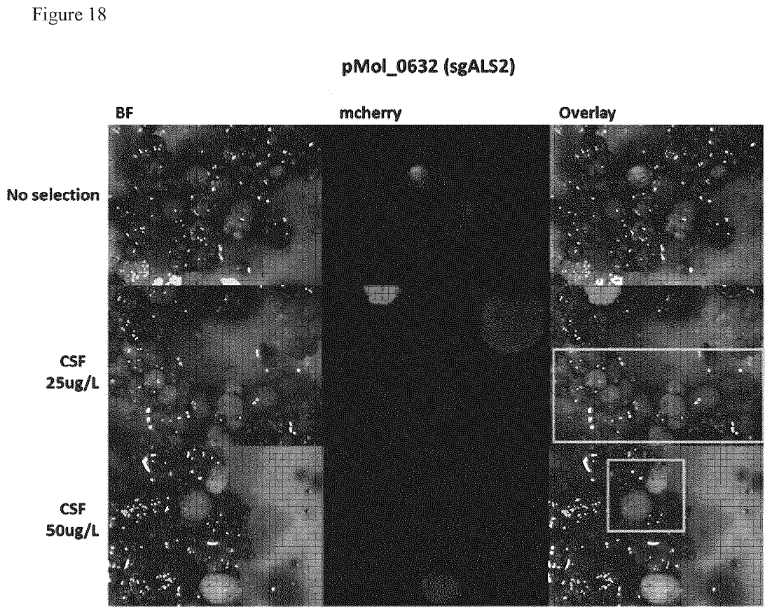

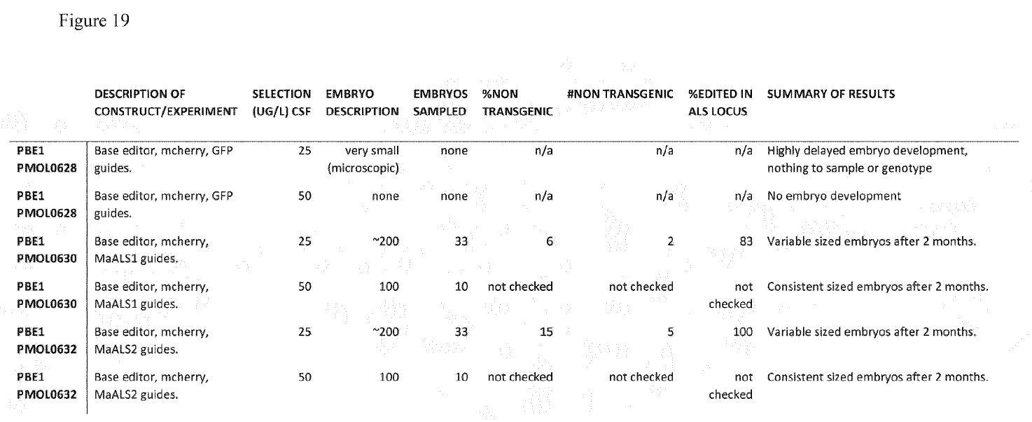

The mcherry fluorescence was measured to assess the ratio of embryos with or without integrated T-DNA (in the genome). Several pictures were taken for all the conditions and a high number of mcherry embryos were observed, roughly 90% of the embryos (see FIGS. 17 and 18). These data indicate that approximately 10% of the embryos transiently express the T-DNA and the editing machinery. To support this observation, qPCR genotyping was performed to detect the presence of the T-DNA.

To test the resistance of ALS-edited banana ECS and embryos we transformed banana ECS cells using Agrobacterium strain AGL1, to transfer T-DNA from plasmids pMOL0628, pMOL0630 and pMOL0632. We also generated an untransformed control sample of cells. ECS cells were co-cultivated with the AGL1 strains for 3 days, then recovered on CS media plus cefotaxime (300 mg/L) for 7 days. Following this period, cells were exposed to different selective embryo development media: EDM+cefotaxime 300, EDM+cefotaxime 300+chlorsulfuron (25 μg/L) or EDM+cefotaxime 300+chlorsulfuron (50 μg/L). Plates containing the cells were incubated statically in the dark at 25° C., and the media was changed every 2 weeks. After 3 months, we made the following observations.

TABLE 10

| Embryos per | | | | Untrans- |
|---|---|---|---|---|
| transfor- | pMOL0628 | pMOL0630 | pMOL0632 | formed |
| mation | (control) | (ALS1 PBE) | (ALS2 PBE) | cells |
| No | 1000 + | 1000 + | 1000 + | 1000 + |
| Selection | embryos | embryos | embryos | embryos |
| CSF25 | No | 100-200 | 100-200 | No |
| | embryos | embryos | embryos | embryos |
| CSF50 | No | 100-200 | 100-200 | No |
| | embryos | embryos | embryos | embryos |

From these data, it was clear that ALS1 and ALS2 PBE constructs pMOL0630 and pMOL0632, respectively, were effective in creating CSF-resistant embryos. We monitored the mCherry fluorescence of these embryo populations and could see that around 90% of regenerated embryos were strongly fluorescent. We believe this means they were transgenic and had integrated the T-DNA containing the PBE and mcherry cassette.

For the pools of embryos that had survived CSF treatment, we needed to correlate survival on CSF with editing in the ALS1/ALS2 loci. To achieve this, we sampled a 96-well plate of embryos and performed Amplicon sequencing to observe the editing frequency in the embryos, and performed qPCR to measure the proportion of embryos that were transgenic and had integrated the T-DNA from the construct.

Samples of embryos were taken from:
Unselected (no CSF) embryos transformed with pMOL0630
Unselected (no CSF) embryos transformed with pMOL0632
Selected (25 μg/L CSF) embryos transformed with pMOL0630
Selected (25 μg/L CSF) embryos transformed with pMOL0632
WT embryos

Purified genomic DNA samples were used as templates in PCR reactions using ALS1 and ALS2 primers as before: ALS12364 and 2365; ALS2 2366 and 2367.

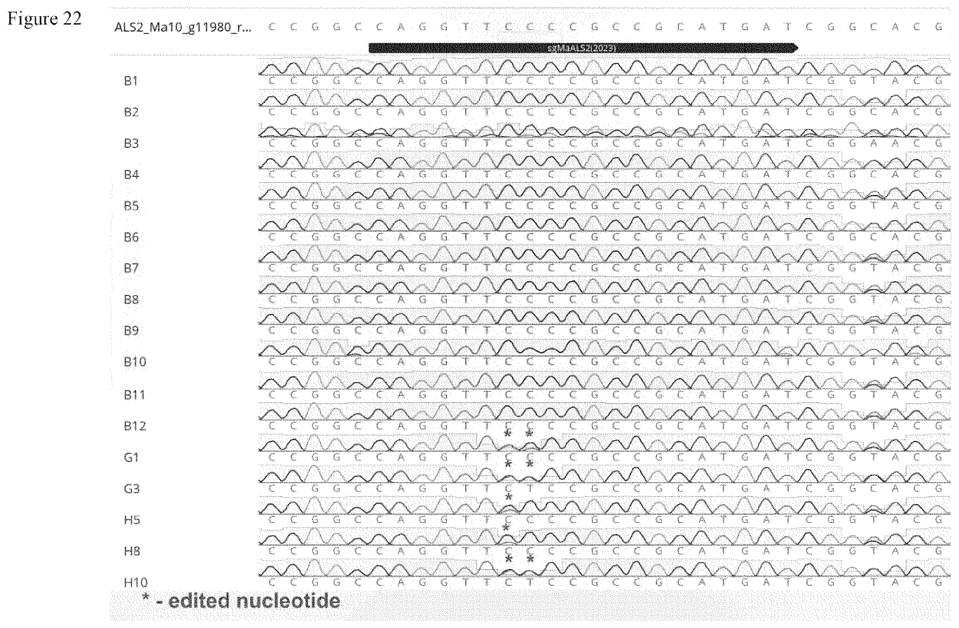

We found ALS editing in every embryo tested except for one embryo. These data are summarised in the table in FIG. 21.

To ascertain whether these embryos were transgenic, we used qPCR on the gDNA samples, using primer sets that bind to the T-DNA. We used primers 3339 and 3340 to detect the Cas9-PBE fusion, and 2537 and 2538 to detect mCherry. We also amplified ALS1 from every sample and used the amplification Ct value of this amplicon as a control (genomic copy number normalisation).

Reactions were all set up with 5 μl of template DNA. We used two WT (wild type) embryo DNA controls, and a water control.

We used Applied PowerUp SYBR Green master mix to perform PCR in the Roche LightCycler96, according to the manufacturer's recommendations.

### Cycle Parameters:

UDG activation (hot-start) 50° C. 2 mins
Dual lock DNA polymerase 95° C. 2 mins
40 cycles of:
Denature 95° C. 15 seconds
Anneal 58° C. 15 seconds
Extend 72° C. 1 minute

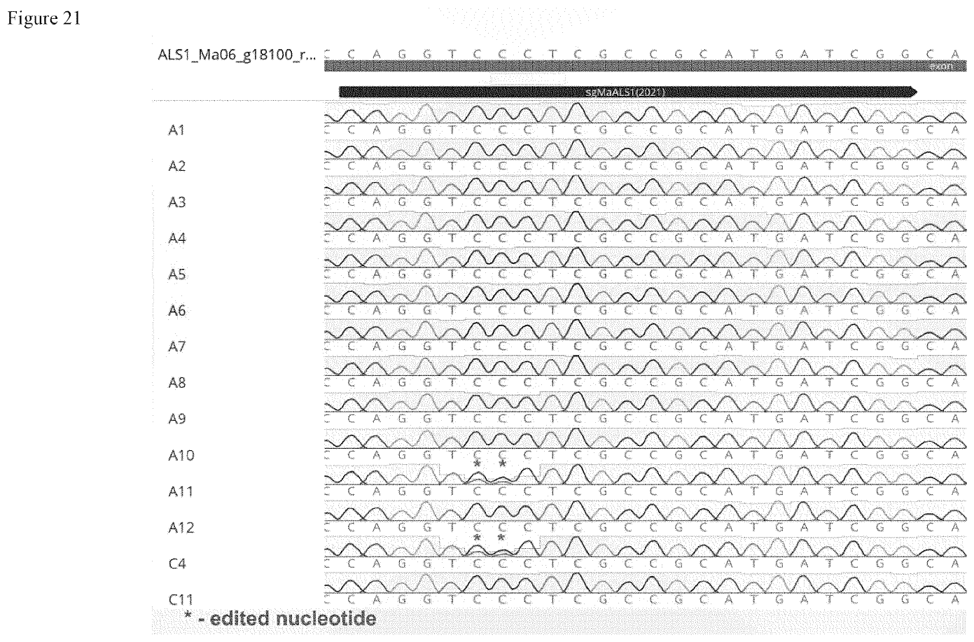

Raw data was analysed using Roche Lightcycler 96 software, and Ct values for each sample were collected, and exported to excel. Here, we normalised the Ct values for the T-DNA amplicons, using the ALS1 primer Ct levels. Once normalised, we plotted the data as shown in FIG. 20. Here, we observed all but one of the unselected embryos appeared to have similar Ct values to the WT samples (8 WT samples per plate, refer to FIG. 20). We set an arbitrary threshold level for the CT value, above which we assume the embryo sample is non-transgenic. Embryo tissue is collected from plates which have significant cellular debris, dead Agrobacterium cells and other, non-developed ECS cells present. We do not expect to observe ‘clean’ qPCR results using this method and are satisfied that a high Ct value (<˜25) is indicative of an embryo being non-transgenic.

We sequenced any candidate transiently edited (non-transgenic) embryos. Sample numbers C4 for ALS1, and samples G1, G3, H5, H8 and H10 for ALS2 (highlighted in FIG. 20 with asterisks).

The target genes were amplified with the primers:
ALS1 2364 and 2365
ALS2 2366 and 2367
Q5 master mix polymerase, 35 cycles. Standard amplification conditions.
PCR products were purified and sent for Sanger sequencing with the same primers used to amplify the fragments.

### Summary of Sequencing Results

### ALS1 (FIG. 21)

### Unselected Embryos

Of the unselected embryos, 10 were WT at the guide site (A1-A10) and one had a 2 nt (contiguous) C-T change at the guide site (A11). One embryo was WT control and was WT in sequence (A12).

### Others (Non-Transgenic Embryos Only)

One (non-transgenic)—edited 2 nt change, C-T in guide site (C4)
One (non-transgenic)—WT (the only non-edited, CSF selected embryo we have identified to date) (C11)

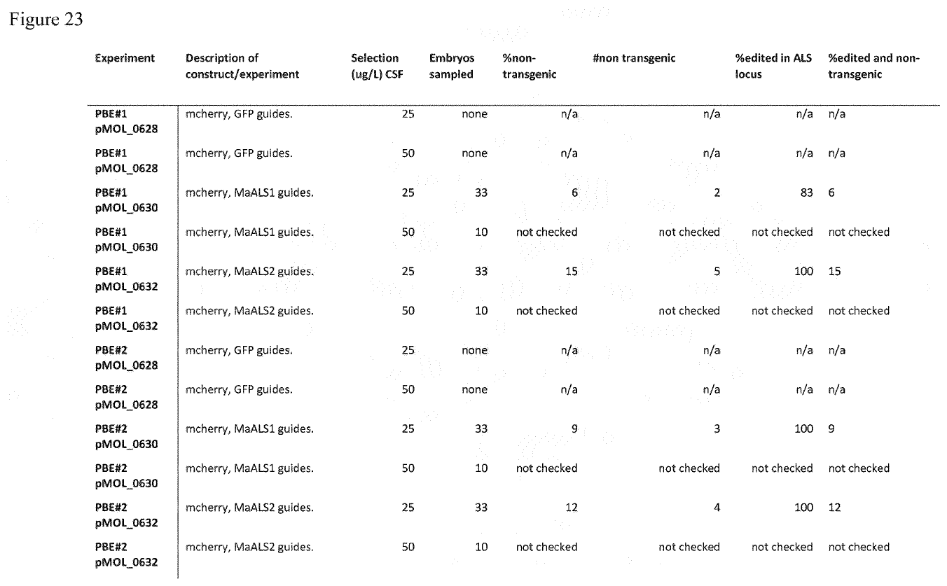

### ALS2 (FIG. 22)

### Unselected Embryos

All WT at guide site (B1-B12)

### Others (Non-Transgenic Embryos Only)

H5—single C-T edit in guide site.
H8—single C-T edit in guide site.
H10—2 nt C-T edit in guide site.
G1—2 nt C-T edit in guide site.
G3—2 nt C-T edit in guide site.

### Conclusions

This Example and the above data provide a number of key observations. Firstly, it is shown that banana cells, in particular ECS cells, can be edited using base editors expressed from T-DNA constructs. Secondly, editing either of the endogenous ALS genes in banana can provide resistance to ALS inhibitors, and treatment of embryos grown from transformed ECS cells with an ALS inhibitor can effectively select for embryos that are edited at the endogenous ALS genes. Thirdly, a significant proportion of the selected embryos do not have the T-DNA construct integrated in their genome, and so they express the base editing machinery transiently and are non-transgenic. Fourthly, non-transgenic embryos can be discerned by observing the absence of expression of a selectable marker present on the T-DNA construct.

### Example 7: Banana Embryos Grown from ECS Cells and Co-Edited in ALS/ALS2 and a Target Gene of Choice can be Identified and Selected Using CSF

To test the ability of ALS based CSF selection to also select for ‘co-editing’ of another trait loci, we designed constructs containing verified ALS sgRNAs, the base editor, and new trait sgRNAs targeting banana genes.

We chose to target two banana traits, ACO (1-Aminocyclopropane-1-Carboxylic Acid Oxidase) and PPO (polyphenol oxidase). The target loci in banana are: ACO1, Ma01_g11540 and PPO2, Ma07_g03540.

We designed sgRNAs to introduce STOP codons as nonsense mutations in the CDS (coding sequence) of the ACO1 and PPO2 loci. We used RGEN tools BE-Designer (http://www.rgenome.net/be-designer/) to design sgRNA sequences (CRISPR RGEN Tools, n.d.; Hwang et al., BMC Bioinformatics 19, 542 (2018).), and these were validated in Geneious Prime.

PPO2:
>Ma07_g03540-CRISPR guide 2961
SEQ ID NO: 21
AGAGCCAGCTGTTGTGGACTTGG
>Ma07_g03540-CRISPR guide 2962
SEQ ID NO: 22
GTTCCAGAAAGGGAGCGAGAAGG
>Ma07_g03540-CRISPR guide 2963
SEQ ID NO: 23
TGAACAGCCTAACCOTGGGGGGG
ACO1:
>ACO_Ma01 g11540-CRISPR guide 2965
SEQ ID NO: 24
GAAGCAAAAGTTCAAACAGTTGG
>ACO_Ma01_g11540-CRISPR guide 2966
SEQ ID NO: 25
CTGCGAGAACCTOGGOCTGGAGG
>ACO_Ma01_g11540-CRISPR guide 2967
SEQ ID NO: 26
GGTGCCAAAGTTCGGACCTTTGG

These sgRNAs were incorporated into Golden Gate constructs to test alongside the validated ALS1 and ALS2 sgRNAs sg2021 and sg2023:

TABLE 11
Plasmids chosen to test

| Plasmid | Description |
|---|---|
| pMOL_0925 | - pNOS::Kan::tOCS - pCsVMV::nCas9PBE1::tg7 - Dummy - |
| (control) | pTaU6::sg1GFP_58 - pTaU6::sg2GFP_59 |
| pMOL_0926 | - pNOS::Kan::tOCS - pCsVMV::nCas9PBE1::tg7 - taU6::MaALS1sg2_2021 - |
| | pTaU6::sg1PPO2_2961 - pTaU6::sg2PPO2_2962 - pTaU6::sg3PPO2_2963 |
| pMOL_0927 | - pNOS::Kan:tOCS - pCsVMV::nCas9PBE1::tg7 - taU6::MaALS1sg2_2021 - |
| | pTaU6::sg3PPO2_2963 - pTaU6::sg4PPO2_2964 |
| pMOL_0928 | - pNOS::Kan::tOCS - pCsVMV::nCas9PBE1::tg7 - taU6::MaALS1sg2_2021 - |
| | pTaU6::sg1ACO1_2965 - pTaU6::sg2ACO1_2966 - pTaU6::sg3ACO1_2967 |
| pMOL_0929 | - pNOS::Kan::tOCS - pCsVMV::nCas9PBE1::tg7 - taU6::MaALS2sg2_2023 - |
| | pTaU6::sg1PPO2_2961 - pTaU6::sg2PPO2_2962 - pTaU6::sg3PPO2_2963 |
| pMOL_0930 | - pNOS::Kan::tOCS - pCsVMV::nCas9PBE1::tg7 - taU6::MaALS2sg2_2023 - |
| | pTaU6::sg3PPO2_2963 - pTaU6::sg4PPO2_2964 |
| pMOL_0931 | - pNOS::Kan::tOCS - pCsVMV::nCas9PBE1::tg7 - taU6::MaALS2sg2_2023 - |
| | pTaU6::sg1ACO1_2965 - pTaU6::sg2ACO1_2966 - pTaU6::sg3ACO1_2967 |
| pMOL_0934 | sgALS1 - pNOS::Kan::tOCS - pCsVMV::nCas9PBE1::tg7 - |
| | taU6::MaALS1sg2_2021 - pTaU6::sg1GFP_58 - pTaU6::sg2GFP_59 |
| pMOL_0935 | - pNOS::Kan::tOCS - pCsVMV::nCas9PBE1::tg7 - taU6::MaALS2sg2_2023 - |
| | pTaU6::sg1GFP_58 - pTaU6::sg2GFP_59 |

We transformed ECS as described previously in Example 6, using the new co-editing plasmids detailed in Table 11 above. Embryos were regenerated as detailed previously, either on media without selection, with 25 mg/L G418, 25 μg/L CSF or 50 μg/L CSF. Initial genotyping of all plasmids tested:

TABLE 12
% Trait editing listed by plasmid in selected embryos

| | G418 | 25CSF | 50CSF | TRAIT 1 | TRAIT 2 |
|---|---|---|---|---|---|
| PMOL_0926 | 75 | 75 | 75 | PPO2 | ALS1 |
| PMOL_0927 | 25 | 0 | 25 | PPO2 | ALS1 |
| PMOL_0928 | 25 | 75 | 100 | ACO1 | ALS1 |
| PMOL_0929 | 25 | 75 | 75 | PPO2 | ALS2 |
| PMOL_0930 | 0 | 0 | 0 | PPO2 | ALS2 |
| PMOL_0931 | 25 | 75 | 100 | ACO1 | ALS2 |

TABLE 13
Trait editing efficiency in embryos by selection method

| | G418 | 25CSF | 50CSF |
|---|---|---|---|
| PPO2 embryos tested | 16 | 16 | 16 |
| PPO2 edited embryos total | 5 | 6 | 7 |
| Percentage edited PPO2 embryos | 31.25 | 37.5 | 43.75 |
| ACO1 embryos tested | 6 | 8 | 7 |
| ACO1 edited embryos total | 2 | 6 | 7 |
| Percentage edited ACO1 embryos | 33.33 | 75 | 100 |

### Example 8—Genotype of Chlorsulfuron (CSF) Resistant Banana Shoots and Embryos Following Transformation with Base-Editing Constructs Targeting ALS1, ALS2, PPO2 and ACO1

### Summary of Results and Discussion

This Example demonstrates that, following transformation with a base editor and gRNA targeting ALS only and selection with an ALS inhibitor, over 99% of all sequenced CSF-selected samples were edited in ALS1 or ALS2. Of these 6-9% were non transgenic and edited in ALS1 and 12-15% of embryos were non-transgenic and edited in ALS2. Out of 36 shoots, 21 (58%) were transgenic and 100% of them were edited in either ALS1 or ALS2. 1/36 samples (3%) was non-transgenic (no T-DNA integration) and was likely edited in a single allele of ALS2.

Following transformation with a base editor and gRNA targeting ALS and “trait” genes ACO1 and PPO2, high levels of co-editing were observed. Up to 5% non-transgenic, stop codon edits were observed in the 376 embryos tested. At the shoot stage, 2/34 samples (6%) were non transgenic (no T-DNA integration). Of the two non-transgenic shoots, one was edited only in ALS2 and the other in ALS2 and PPO2. We therefore find that in banana shoots, there is a non-transgenic, trait edited frequency of 3%.

### Materials and Methods

We performed two distinct groups of experiments.
1. Baselining ALS editing frequency and chlorsulfuron resistance in banana embryos and shoots using a cytidine base editor (“CBE”) and sgRNAs targeting either ASL1 or ALS2. We will refer to these as experiments PBE #1 (Grand Naine, GN236) and PBFA2 (G251).
2. Assessing the feasibility of creating ‘co-editing’ in banana embryos and shoots using the same CBE and sgRNAs targeting both ALS (1 or 2) and a trait gene (PPO2 or ACO1), referred to as experiment PBE #3.
   We transformed banana ECS with the following constructs:

### PBE #1 and PBE #2

### Plasmids

All with CsVMV-rAPOBEC1 CBE (rAPOBEC+nCas9 (D10A) PBE from Zong et al. 2017*)
pMol_0628 (mcherry, sgGFP--CsVMV::mCherry::tg7-pCsVMV::nCas9PBE1::tg7-Dummy-pTaU6::sg1GFP_58
pMol_0630 (mcherry, sgALS1--CsVMV::mCherry::tg7-pCsVMV::nCas9PBE1::tg7-taU6::MaALS1sg2_2021)
pMol_0632 (mcherry, sgALS2--CsVMV::mCherry::tg7-pCsVMV::nCas9PBE1::tg7-taU6::MaALS2sg2_2023)
   Zong Y, Wang Y, Li C, Zhang R, Chen K, Ran Y, Qiu J L, Wang D, Gao C. Precise base editing in rice, wheat and maize with a Cas9-cytidine deaminase fusion. Nat Biotechnol. 2017 May; 35(5):438-440. doi: 10.1038/nbt.3811. Epub 2017 Feb. 27. PMID: 28244994.

### PBE #3

### Plasmids

All with CsVMV-rAPOBEC1 CBE (rAPOBEC+nCas9 (D10A) PBE from Zong et al. 2017*) and nosP-NptII
pMol_0925 (sgGFP-pNOS::Kan::tOCS-pCsVMV::nCas9PBE1::tg7-Dummy-pTaU6::sg1GFP_58-pTaU6::sg2GFP_59)
pMol_0934 (sgGFP, sgALS1-pNOS::Kan::tOCS-pCsVMV::nCas9PBE1::tg7-taU6::MaALS1sg2_2021-pTaU6::sg1GFP_58-pTaU6::sg2GFP_59)
pMol_0935 (sgGFP, sgALS2-pNOS::Kan::tOCS-pCsVMV::nCas9PBE1::tg7-taU6::MaALS2sg2_2023-pTaU6::sg1GFP_58-pTaU6::sg2GFP_59)
pMol_0926 (sgPPO2, sgALS1-pNOS::Kan::tOCS-pCsVMV::nCas9PBE1::tg7-taU6::MaALS1sg2_2021-pTaU6::sg1PPO2_2961-pTaU6::sg2PPO2_2962-pTaU6::sg3PPO2_2963)
pMol_0927 (sgPPO2, sgALS1-pNOS::Kan::tOCS-pCsVMV::nCas9PBE1::tg7-taU6::MaALS1sg2_2021-pTaU6::sg3PPO2_2963-pTaU6::sg4PPO2_2964)
pMol_0928 (sgACO1, sgALS1-pNOS::Kan::tOCS-pCsVMV::nCas9PBE1::tg7-taU6::MaALS1sg2_2021-pTaU6::sg1ACO1_2965-pTaU6::sg2ACO1_2966-pTaU6::sg3ACO1_2967)
pMol_0929 (sgPPO2, sgALS2-pNOS::Kan::tOCS-pCsVMV::nCas9PBE1::tg7-taU6::MaALS2sg2_2023-pTaU6::sg1PPO2_2961-pTaU6::sg2PPO2_2962-pTaU6::sg3PPO2_2963)
pMol_0930 (sgPPO2, sgALS2-pNOS::Kan::tOCS-pCsVMV::nCas9PBE1::tg7-taU6::MaALS2sg2_2023-pTaU6::sg3PPO2_2963-pTaU6::sg4PPO2_2964)
pMol_0931 (sgACO1, sgALS2-pNOS::Kan::tOCS-pCsVMV::nCas9PBE1::tg7-taU6::MaALS2sg2_2023-pTaU6::sg1ACO1_2965-pTaU6::sg2ACO1_2966-pTaU6::sg3ACO1_2967)
   ECS were left to ‘recover’ after transformation for 1 week in CS+cefo 300 mg/L.
   We regenerated embryos in 4 different conditions:
1. No selection
2. Stable G418 selection (in EDM only 2-3 months) from PBE #3 only (PBE #1/2 plasmids only have mCherry, not NptII)
3. 25 μg/L chlorsulfuron (in EDM only 2-3 months)
4. 50 μg/L chlorsulfuron (in EDM only 2-3 months)

We assessed selection efficacy by CSF for each plasmid by observing embryo regeneration.

For PBE #1 and PBE #3 we only observed embryo development on CSF when the construct contained the base editor and an sgRNA targeting ALS1 or ALS2. There was reproducibly more than 200 embryos on every single transformation plate.

We genotyped at two distinct timepoints in development:
Single mature embryos after EDM stage
Single green leaf tips from plantlets in RM media

### Genotyping Methods and Results

gDNA from embryos and plantlets was extracted and qPCR was used to test for the presence of integrated tDNA in the plant genome. Three independent qPCRs were run on each sample with different sets of primers (listed below) spanning the tDNA, ranging from the left to right border. An additional qPCR was run on an endogenous banana gene (actin) for normalisation of Ct values. All qPCRs were run on the LightCycler® 96 System (Roche) using PowerUp™ SYBR® Green Master Mix (Thermo Fisher Scientific™).

For edit validation, amplicons for each target gene (als1, als2, ppo2, aco1) were made using Q5® High-Fidelity 2× Master Mix (New England Biolabs, NEB) using reaction settings listed below. PCR products were purified using Monarch® PCR & DNA Cleanup Kit (NEB) according to manufacturer's specifications. The amplicons were after validated by Sanger sequencing.

TABLE 14
List of primers used for banana embryos and shoots genotyping.

| | | | | SEQ ID |
|---|---|---|---|---|
| Number | Gene | Type | Sequence | NO |
| 2644 (G0025) | ACO1 Fw | Amplicon | ACCCAAAGGTCGACGACAAA | 27 |
| 3019 (G0026) | ACO1 | Amplicon | CATGAACGCCCTCATGTTGTT | 28 |
| | Rev | | | |
| 3022 | ACO1 Fw | Sequencing | TTGATGCCCACGAGCATTTGA | 29 |
| 1757 | ACO1 | Sequencing | ATCCACTGGCCATCCTTGA | 30 |
| | Rev | | | |
| 2508 (G0163) | PPO2 Fw | Amplicon/Sequencing | AGGTGCCCATGTTCTCGAAG | 31 |
| 2509 (G0164) | PPO2 Rev | Amplicon/Sequencing | AAGTAGGTCACGTCCCTCCC | 32 |
| 2364 (G0117) | ALS1 Fw | Amplicon/Sequencing | TCCATCAGGCTCTCACCCG | 33 |
| 2365 (G0118) | ALS1 Rev | Amplicon/Sequencing | CCACCACCAACATAGAGGACT | 12 |
| 2366 (G0124) | ALS2 Fw | Amplicon/Sequencing | ATCTGTTCGCCTACCCTGGA | 13 |
| 2367 (G0125) | ALS2 Rev | Amplicon/Sequencing | CAACATAGAGAACCGGGCGA | 34 |
| 277 (G0390) | Actin Fw | qPCR genotyping | ACCGAAGCCCCTCTTAACCC | 35 |
| 278 (G0391) | Actin Rev | qPCR genotyping | GTATGGCTGACACCATCACC | 36 |
| G0339 | NptII Fw | qPCR genotyping | CAGACAATCGGCTGCTCTGA | 37 |
| G0340 | NptII Rev | qPCR genotyping | GATGTTTCGCTTGGTGGTCG | 38 |
| 3339 (G0418) | nCas9 | qPCR genotyping | CCTCGTTGCTTGGGGAGTAG | 39 |
| | front Fw | | | |
| 3340 (G0419) | nCas9 | qPCR genotyping | GCCATCACCGAGTTCCTCAG | 40 |
| | front Rev | | | |
| 2537 (G0448) | nCas9 | qPCR genotyping | ATCGACCTCTCACAACTCGG | 41 |
| | back Fw | | | |
| 2538 (G0449) | nCas9 | qPCR genotyping | TTGTTGCCGATGACCTCCTC | 42 |
| | back Rev | | | |

TABLE 15
Sanger Amplicon Reaction Setup

| | | |
|---|---|---|
| Q5 High-Fidelity 2X | 12.5 | μL |
| Master Mix | | |
| Forward Primer | 1.25 | μL |
| Reverse Primer | 1.25 | μL |
| Template DNA | 3 | μL |
| H2O | 7 | μL |
| Total | 25 | μL |

TABLE 16

| | | |
|---|---|---|
| Temperature | Time | Cycles |

| | | | | | |
|---|---|---|---|---|---|
| Initial Denaturation | 98° | C. | 30 | sec | |
| Polymerase activation | 98° | C. | 10 | sec | 35 |
| Denaturation | 50-72° | C. | 15 | sec | |
| Annealing/Extension | 72° | C. | 10-60 | sec | |
| Final Extension | 72° | C. | 2 | min | |

| | | |
|---|---|---|
| 4° | C. | Hold |

TABLE 17
qPCR Reaction Setup

| | | |
|---|---|---|
| qPCR Powerup MM | 12.5 | μL |
| 2X | | |
| FW/Rev Primer mix | 1.25 | μL |
| Template DNA | 4 | μL |
| H20 | 7.25 | μL |
| Total | 25 | μL |

TABLE 18

| qPCR primers: | |
|---|---|
| Actin (normalisation) | 277,278 (G0390, G0391) |
| NptII | G0339, G0340 |
| nCas9 front | (G0418, G0419) |
| nCas9 back | (G0448, G0449) |

TABLE 19
qPCR Genotyping Settings

| | Temperature | Time | | Cycles | Acquisition |
|---|---|---|---|---|---|
| UDG activation | 50° C. | 2 | min | | |
| Polymerase activation | 95° C. | 2 | min | | |
| Denaturation | 95° C. | 15 | sec | 40 | Continuous |
| Annealing/Extension | 60° C. | 1 | Min | | |
| High resolution | 95° C. | 1 | min | | |
| melting | 40° C. | 1 | min | | |
| | 65° C. | 1 | sec | | |
| | 97° C. | 1 | sec | | |

### PBE #1 and PBE #2 Embryo Genotyping Data (ALS Only, No Trait)

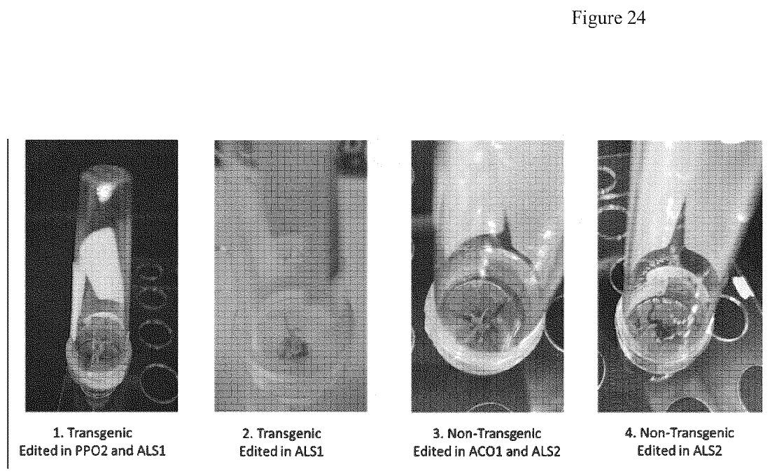

We found between 6-9% of embryos were non-transgenic and edited in ALS1 and 12-15% of embryos were non-transgenic and edited in ALS2. Over 99% of all sequenced CSF selected samples were edited in ALS1 or ALS2. We only fully assessed 25 μg/L CSF in this genotyping experiment. These data are presented in FIG. 23.

### PBE #1 Shoot Genotyping Data (ALS Only, No Trait)

Embryogenic cell suspensions (ECS) were transformed with an Agrobacterium strain harbouring plasmids encoding the nCas9 machinery and expressing sgRNAs. Agrobacterium transformation is performed according to Kanna et al., Molecular Breeding October 2004, Volume 14, Issue 3, pp 239-252. Embryogenic cells are co-cultivated with Agrobacterium for one to three days, and then transferred to embryo development medium (EDM) containing CSF and then a maturation medium without CSF (relevant media can be found, for example, in Strosse H., R. Domergue, B. Panis, J. V. Escalant and F. Côte, 2003, Banana and plantain embryogenic cell suspensions (A. Vézina and C. Picq, eds). INIBAP Technical Guidelines 8, The International Network for the Improvement of Banana and Plantain, Montpellier, France). The mature embryos are germinated in germination medium (Strosse H., R. Domergue, B. Panis, J. V. Escalant and F. Côte. 2003. Banana and plantain embryogenic cell suspensions (A. Vézina and C. Picq, eds). INIBAP Technical Guidelines 8. The International Network for the Improvement of Banana and Plantain, Montpellier, France) and young shoots are transferred to shoot maturation medium until approximately 1 cm in height. Shoots are transferred to rooting medium for plantlet development.

Out of 36 shoots, 21 (58%) were transgenic and 100% of them were edited in either ALS1 or ALS2. 1/36 samples (3%) was non-transgenic (no T-DNA integration) and was likely edited in a single allele of ALS2.

TABLE 20

| | | | | | TRANS- | | EDITING | |
|---|---|---|---|---|---|---|---|---|
| | | | | | GENIC | | FROM | NON- |
| | | | SELEC- | NO. OF | SHOOTS | | TRANS- | TRANSGENIC |
| EXPERI- | | TARGET | TION | SHOOTS | FROM CSF | | GENIC CSF | EDITED |
| MENT | PLASMID | GENE | CONC. | SAMPLED | SELECTION | | SELECTION | SHOOTS |
| PBE#1 - | pMOL_0630 | ALS1 | 25CSF | 11 | 6/11 | (55%) | 1/1 tested | 0/5 - 0% |
| G-0001 | | | | | | | (100%) | |
| | | ALS1 | 50CSF | 3 | 2/3 | (66%) | 1/1 tested | 0/1 - 0% |
| | | | | | | | (100%) | |
| | pMOL_0632 | ALS2 | 25CSF | 13 | 9/13 | (69%) | 1/1 tested | ¼ - 25% |
| | | | | | | | (100%) | |
| | | ALS2 | 50CSF | 9 | 4/9 | (45%) | 1/1 tested | 0/5 - 0% |
| | | | | | | | (100%) | |

### PBE #3 Genotyping Data (ALS and Trait Gene ‘Coediting’)

We found high levels of co-editing in ACO1 and PPO2 following CSF selection. We observed up to 5% non-transgenic, stop codon edits in the 376 embryos tested.

We performed an initial screen of the editing frequencies in a small sample of embryos from each of the 8 plasmids used in this experiment. From this, we determined that pMOL_0926 and pMOL0931 showed some of the highest editing efficiency. We sampled 4×96 well plates of embryos regenerated following transformation with these two plasmids—from both 25 and 50 μg/L CSF selection (4×96-well plates in total).

TABLE 21
Initial screen of plasmid editing efficiency in embryos (%):

| Plasmid | Trait 1 | Marker | G418 | 25CSF | 50CSF |
|---|---|---|---|---|---|
| pMol_0926 | PPO2 | ALS1 | 75 | 75 | 75 |
| pMol_0927 | PPO2 | ALS1 | 25 | 0 | 25 |
| pMol_0928 | ACO1 | ALS1 | 25 | 75 | 100 |
| pMol_0929 | PPO2 | ALS2 | 25 | 75 | 75 |
| pMol_0930 | PPO2 | ALS2 | 0 | 0 | 0 |
| pMol_0931 | ACO1 | ALS2 | 25 | 75 | 100 |

Percentage editing in trait gene (PPO2 or ACO1), when selected by either G418 or CSF. (n4 for each datapoint). Frequencies appear to be construct specific, e.g. pMOL_0927 only had PPO2 edits in 2/12 embryos tested, whereas pMOL-0926 resulted in 9/12 PPO2 edited embryos. There was marked difference in sgRNA efficiencies too (data not shown).

TABLE 22
Large scale genotyping of embryos regenerated following transformation
with pMOL0926 or pMOL0931 only, using 25 or 50 μg/L CSF selection.

| | | | Selection | | | % edited | % edited | #edited |
|---|---|---|---|---|---|---|---|---|
| | Sample | # of | (ug/L) | % non | #non | in ALS | and non | and non |
| Plasmid | Type | samples | CSF | transgenic | transgenic | locus | transgenic | transgenic |
| pMOL_926 | Embryo | 94 | 25 | 11.7 | 11 | 100 | 5 | 5 |
| pMOL_926 | Embryo | 94 | 50 | 25 | 24 | 100 | 3 | 3 |
| pMOL_931 | Embryo | 94 | 25 | 17 | 16 | 100 | 1 | 1 |
| pMOL_931 | Embryo | 94 | 50 | 11.7 | 11 | 100 | 7 | 7 |

PBE #3 (embryos) pMOL_926
CSF25:
   Out of 94 samples, 11 (11.7%) were non-transgenic (no T-DNA integration).
   5% (5 non-transgenic embryos) had stop codon-generating edits in PPO2.
   All transgenic samples sequenced (7) had stop codon generating edits in the PPO2 trait gene at both sg2961 and sg2962.
CSF50:
   Out of 94 samples, 24 (25.5%) were non-transgenic (no T-DNA integration)
   3% (3 non-transgenics) had stop codon-generating edits in PPO2.
   A single transgenic sample sequenced had a stop codon-generating edit in the PPO2 locus at sg2962.
      PBE #3 (Embryos) pMOL_931:
CSF25:
   Out of 94 samples, 16 (17%) were non-transgenic (no T-DNA integration).
   1% (1 non-transgenic) had a stop codon-generating edit in the ACO1 trait gene at the sg2967 locus.
   Two transgenic samples sequenced had stop codon-generating edits in the ACO1 trait gene at the sg2967 locus.
CSF50:
   Out of 94 samples, 11 (11.7%) were non-transgenic (no T-DNA integration)
   7 non-transgenic embryos had a stop codon-generating at the guides in the ACO1 trait gene
   Two transgenic samples sequenced had stop codon-generating edits in the ACO1 trait gene at the sg2967 locus.

### PBE #3 Shoot Genotyping Data (ALS and Trait ‘Coediting’)

2/34 samples (6%) were non transgenic (no T-DNA integration). Of the two non-transgenic shoots, one was edited only in ALS2 and the other in ALS2 and PPO2. We therefore find a non-transgenic, trait edited frequency of 3% from this experiment. Genotyping the pMOL0927 and pMOL0930 shoots and embryos was not completed.

TABLE 23
Genotyping of banana shoots regenerated following transformation with
pMOL_0926-pMOL_0931 using 25 or 50 ug/L CSF selection.

| | | | Selection | | | % edited | % edited | #edited in trait |
|---|---|---|---|---|---|---|---|---|
| | Sample | # of | (ug/L) | % non | #non | in ALS | and non | gene and non |
| Plasmids | Type | samples | CSF | transgenic | transgenic | locus | transgenic | transgenic |
| pMol_926- | Shoots | 34 | 25/50 | 6 | 2 | 100 | 3 | 1 |
| pMol_931 | | | | | | | | |

From the 34 shoots sampled, 100% were found to have edits in either ALS1 or ALS2.
53% (17) were found to have stop codon-generating edits in the trait gene.
2/34 samples (6%) were non transgenic (no T-DNA integration).
Of the two non-transgenic shoots, one was edited only in ALS2, one in ALS2 and PPO2.
3% transient co-editing frequency overall in the first 34 shoots tested.

### Shoot Count and Health Data

Here we report the numbers of shoots generated and collected from experiments PBE #1 (ALS only) and PBE #3 (ALS and trait ‘coediting’). We report numbers of plant samples (‘cones’) that were lost due to contamination or death from other causes.

### PBE #1 Shoot Regeneration Frequency (ALS Only)

We observe a lower number of shoots from 50 μg/L CSF (17 and 37) than from 25 μg/L CSF (36 and 61) for ALS1 and ALS2 targets (respectively). Overall ALS2 plasmid transformations yield a higher number of shoots (61 and 37) than ALS1 (36 and 17) from 25 and 50 μg/L of CSF (respectively). We do not see any correlation in plant survival with either the editing (gene) target, or, with the concentration of CSF used for selection.

TABLE 24
Number of shoots generated after CSF selection.

| Plasmid | | | | |
|---|---|---|---|---|
| Number | Gene | | Number of | Number of |
| (pMOL) | Target (s) | Selection | shoots | Cones Lost |
| 630 | ALS 1 | Controls | 44 | 8 |
| | | (unselected) | | |
| | | 25CSF | 36 | 11 |
| | | 50CSF | 17 | 7 |
| 632 | ALS 2 | Controls | 49 | 17 |
| | | (unselected) | | |
| | | 25CSF | 61 | 4 |
| | | 50CSF | 37 | 1 |
| | | totals | 244 | 48 |

### PBE #3 Shoot Regeneration Frequency (ALS and Trait ‘Coediting’)

In contrast to PBE #1, we observe a higher number of shoots from 50 μg/LCSF (148 and 328) than from 25 μg/L CSF (55 and 140) for ALS1 and ALS2 targets (respectively). Consistent with PBE #1, overall ALS2 plasmid transformations yield a higher number of shoots (140 and 328) than ALS1 (55 and 148) from 25 and 50 μg/L of CSF (respectively). We do not see any correlation in plant death (cone loss) with gene target or selection.

There is a highly variable frequency of shoot output from plasmid and selection method.

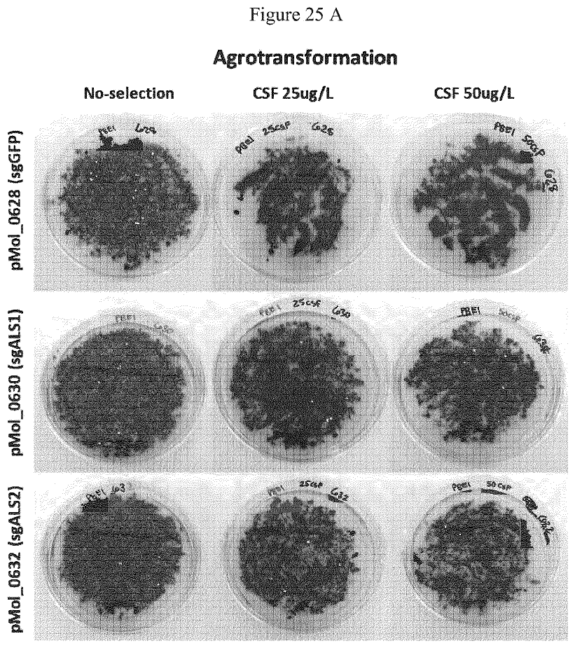

A selection of the different plant genotypes identified in PBE #3 is shown in FIG. 24.
1. A8—Transgenic: Edited in PPO2 and ALS1 (pMol_926)
2. A9—Transgenic: No trait edit. Edited in ALS1 (pMol_927)
3. H12—Suspected non-transgenic. Edited in ACO1 (at two guide sites) and ALS2 (pMol_931)
4. B11—Non-trans genic. Edited in ALS2 (pMol_930)

TABLE 25
Summary table:

| Gene | | Number of | Number of |
|---|---|---|---|
| Target (s) | Selection | shoots | Cones Lost |
| ALS1 | Controls | 9 | 2 |
| | (unselected) | | |
| | 25CSF | 55 | 41 |
| | 50CSF | 148 | 86 |
| | 25G418 | 43 | 23 |
| ALS2 | Controls | 1 | 0 |
| | (unselected) | | |
| | 25CSF | 140 | 87 |
| | 50CSF | 328 | 189 |
| | 25G418 | 88 | 41 |
| Control | Controls | 0 | 0 |
| (pMOL_925) | (unselected) | | |
| | 25CSF | 0 | 0 |
| | 50CSF | 0 | 0 |
| | 25G418 | 4 | 2 |
| | totals | 816 | 471 |

TABLE 26
Complete table of shoot frequencies (3 ECS
transformations per cell in this table):

| Plasmid | | | | |
|---|---|---|---|---|
| Number | Gene | | Number of | Number of |
| (pMol) | Target (s) | Selection | shoots | Cones Lost |
| 925 | N/A (GFP) | Controls | 0 | 0 |
| | | (unselected) | | |
| | | 25CSF | 0 | 0 |
| | | 50CSF | 0 | 0 |
| | | 25G418 | 4 | 2 |
| 926 | ALS1/PPO2 | Controls | 0 | 0 |
| | | (unselected) | | |
| | | 25CSF | 18 | 13 |
| | | 50CSF | 66 | 39 |
| | | 25G418 | 4 | 1 |
| 927 | ALS1/PPO2 | Controls | 2 | 0 |
| | | (unselected) | | |
| | | 25CSF | 9 | 5 |
| | | 50CSF | 47 | 22 |
| | | 25G418 | 11 | 6 |
| 928 | ALS1/ACO1 | Controls | 7 | 2 |
| | | (unselected) | | |
| | | 25CSF | 31 | 23 |
| | | 50CSF | 35 | 25 |
| | | 25G418 | 21 | 12 |
| 929 | ALS2/PPO2 | Controls | 1 | 0 |
| | | (unselected) | | |
| | | 25CSF | 85 | 52 |
| | | 50CSF | 162 | 77 |
| | | 25G418 | 35 | 13 |
| 930 | ALS2/PPO2 | Controls | 0 | 0 |
| | | (unselected) | | |
| | | 25CSF | 34 | 25 |
| | | 50CSF | 126 | 84 |
| | | 25G418 | 8 | 7 |
| 931 | ALS2/ACO1 | Controls | 0 | 0 |
| | | (unselected) | | |
| | | 25CSF | 21 | 10 |
| | | 50CSF | 40 | 28 |
| | | 25G418 | 38 | 19 |
| 934 | ALS1 | Controls | 0 | 0 |
| | | (unselected) | | |
| | | 25CSF | 0 | 0 |
| | | 50CSF | 0 | 0 |
| | | 25G418 | 2 | 0 |
| 935 | ALS2 | Controls | 0 | 0 |
| | | (unselected) | | |
| | | 25CSF | 0 | 0 |
| | | 50CSF | 0 | 0 |
| | | 25G418 | 10 | 2 |
| | | total | 816 | 471 |

In summary, we observe a variety of shoot phenotypes at these early stages of development. Overall, we observe that shoots regenerated without G418 or CSF selection are the healthiest in appearance. There does not appear to be a marked difference in frequency between regenerated shoots from embryo selection using G418 or CSF overall. Crucially, we find healthy shoots from PBE #3 that are transgenic and edited in both ALS and a trait gene. Of the three non-transgenic, trait gene edited shoots from PBE #3, two are healthy plants, and one was lost to contamination.

### Conclusions

These data demonstrate that transformations with the constructs generate >200 embryos per transformation following selection with either 25 or 50 μg/L CSF. Strikingly, these embryos are consistently edited at a high percentage in the ALS loci, and in a trait gene. We observe a non-transgenic rate in CSF selected samples of >10% across all experiments, in embryos and shoots. Overall frequency of non-transgenic, edited shoots is between 3-6% across the PBE1 and PBE3 experiments.

We do not observe any negative effects on shoot health that can be attributed to ALS or trait editing per se. We have examples of healthy shoots that are edited in ALS1 or ALS2 and in trait genes, both non-transgenic and transgenic.

We do see a negative effect on shoot development of selection in general—we applied selection during the EDM stage for both CSF and G418 and see a retardation in shoot development relative to unselected control shoots from the same experiment. This could be mitigated by waiting until shoots are more vigorous before moving them to ‘cones’.

### Example 9—Comparison of Agrobacterium Transformation and Bombardment

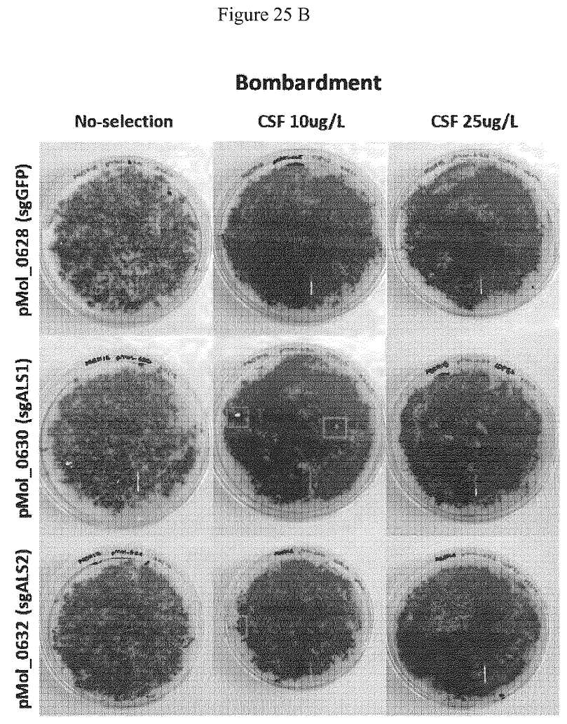

Transformation using agrobacterium and particle bombardment was compared. The data are presented in FIG. 25.

Following transformation with agrobacterium, no embryos with the negative control plasmid developed in the presence of CSF. Hundreds of embryos developing in the presence of CSF were observed from both “resistant” plasmids. Fewer embryos developed in the presence of higher amounts of CSF (50 μg/L).

Following transformation by particle bombardment, again no embryos with the negative control plasmid developed in the presence of CSF. Bombarded cells are more susceptible to treatments so lower concentrations of CSF were used for selection. Only a few embryos developing in the presence of CSF from both “resistant” plasmids were observed (green box). It appears that CSF 25 μg/L was too strong for selection of edited bombarded cells.

### Agrobacterium Tumefaciens Preparation

Agrobacterium tumefaciens AGL1 carrying the binary plasmids pMol_0628, pMol_0630, and pMol_0632 were streaked from glycerol in LB solid medium supplemented with rif 50 mg/L, kan 100 mg/L and carb 50 mg/L and incubated at 28° C. for 2 days. On the second day, the bacteria were inoculated in MG/L media supplemented with the same antibiotics and incubated overnight at 28° C. Next day, the cultures were spun down at 4000 rpm for 10 min to pellet the cells and after they were resuspended in ABIM (Agrobacterium induction media) to induce virulence and incubated overnight in the dark at room temperature. The OD600 of the bacteria was measured using NanoDrop2000 and normalised to 0.6 in CS liquid media supplemented with acetosyringone (100p M).

### Banana ECS Agrotransformation

Banana ECS with cells 2/3 days old was pelleted and resuspended in CS medium. 0.5 mL of the suspension (approximately 0.1 mL of ECS) was split accordingly to the number of transformations into small Eppendorf tubes. The cells were incubated at 45° C. for 5 min and after they had cooled down for another 5 min. Then, 1 mL of agrobacterium suspension was added to the banana ECS. The mixture was mixed well and spun down at 1000 rpm for 5 min, and then, it was incubated for 25 min at room temperature. The banana cells were pooled, and the bacteria suspension was removed. The cells were inoculated in CS plates supplemented with acetosyringone 100 μM for 3 days at room temperature to promote transformation.

### Banana Embryos Developing and Germination

After 3 days, the banana ECS were collected from the previous media and transferred to CS media supplemented with cefotaxime 300 mg/L. One week later, the banana cells were transferred to EDM plates supplemented with cefo 300 mg/L (control), or cefo 300 mg/L and CSF 25 μg/L or cefo 300 mg/L and CSF 50 μg/L. The media was refreshed every 2 weeks until embryos were developed. Subsequently, the banana embryos were transferred to maturation media without CSF (EMM) for a month and then they were transferred to germination medium (GM4) without CSF until shoots developed.

### Example 10—Comparison of Selection Using ALS Inhibitor, G418 or Hygromycin

This Example compared the efficiency achieved when selecting using an ALS inhibitor, G418 or hygromycin. It was found that selection with an ALS inhibitor increases the efficiency of developing/identifying non-transgenic trait-edited banana samples.

Table 27 below shows the frequency of editing events detected in gDNA extracted from individual banana embryos or leaf material. In every case, regenerated material was transformed with constructs containing T-DNA that would generate resistance in positive transformants.

TABLE 27

| | | | | Non- |
|---|---|---|---|---|
| Genotyped | Selection | Transgenic | Editing | transgenic |
| material | method | frequency | frequency | edited |
| Embryos | CSF | 344/408 = 84.3% | 57/63 = 90.5% | 19/584 = 3.2% |
| Shoots | CSF | 53/70 = 75.7% | 34/36 = 94.4% | 3/46 = 6.5% |
| embryos | G418 | 95/943 = 10.1% | 35/867 = 4.0% | 0/774 = 0% |
| | transient | | | |
| embryos | Hygromycin | 67/630 = 10.6% | 4/630 = 0.63% | 0/630 = 0% |
| | transient | | | |
| embryos | G418 stable | 18/28 = 64.3% | 17/28 = 60.7% | — |
| embryos | Hygromycin | 73/103 = 70.9% | 6/14 = 42.9% | — |
| | stable | | | |

We observe that CSF selection is the most efficient method we have used to generate transgenic lines of banana (75-84%) when compared to stable selection with either G418 or hygromycin (64-71%). The editing frequency observed in CSF selected material is 90-94%, whereas we only observe 43-61% editing frequency in stable selection using G418 or hygromycin.

### Example 11—Optimisation of ALS Inhibitor Treatment for Edited Embryos

Data were obtained for using CSF in ECS regeneration (3 months treatment) for concentrations 0, 25, 50 and 100 μg/L.

TABLE 28

| | | Frequency of ALS |
|---|---|---|
| | Frequency of WT | edited embryos |
| Concentration | embryos recovered | recovered after |
| of CSF | after selection | selection |
| 0 | 30000 ± 2000 | 30000 ± 2000 |
| 25 | 0 | 1500 ± 500 |
| 50 | 0 | 750 ± 250 |
| 100 | 0 | 0 |

### Further Sequences

SEQ ID NO:43
ACS—Musa acuminata
>Ma04_g31490.1
SEQ ID NO:44
ACS—Musa acuminata
>Ma04_g35640.1
SEQ ID NO:45
ACS—Musa acuminata
>Ma09_g19150.1
SEQ ID NO:46
ACO1—Musa acuminata
>Ma01_g11540.1
SEQ ID NO:47
ACO—Musa acuminata
>Ma07_g19730.1
SEQ ID NO:48
PPO2 —Musa acuminata
>Ma07_g03540

1. A method of generating at least one banana embryo or plant comprising at least one desired mutation in a target sequence or sequences, the method comprising:
a. contacting a banana cell with at least one agent operable to introduce the at least one desired mutation in the target sequence(s) and at least one agent operable to introduce at least one mutation in at least one endogenous acetolactate synthase (ALS) gene so as to provide resistance to an ALS inhibitor;
b. optionally culturing the banana cell to generate a banana cell population, tissue, or plant; and
c. treating the banana cell, cell population, tissue or plant with an ALS inhibitor to select at least one banana cell, tissue or plant mutated in at least one endogenous acetolactate synthase gene and comprising the desired mutation(s) in the target sequence(s); and
d. generating at least one banana embryo or plant from the banana cell, tissue or plant selected in step (c).

2. The method of claim 1, wherein the banana cell is an embryogenic cell, such as wherein the banana cell is an embryogenic cell in an embryogenic cell suspension and step (a) comprises contacting the embryogenic cell suspension.

3. The method of claim 1, wherein the agents operable to introduce the mutations comprise a base editor and guide RNAs or comprise at least one nucleic acid construct that encodes a base editor and guide RNAs, preferably wherein the base editor is a fusion polypeptide comprising a modified Cas, such as nCas9 or dCas9, and a cytidine deaminase moiety or an adenine deaminase moiety, such as a base editor selected from the group consisting of APOBEC, BE1, BE2, BE3, HF-BE3, BE4, BE4max, BE4-GAM, YE1-BE3, EE-BE3, YE-BE3, YEE-BE3, VOR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, ABE7.10*, xABE, ABESa, VOR-ABE, VRER-ABE, and SaKKH-ABE.

4. (canceled)

5. The method of claim 1, wherein the mutations are introduced via HDR and the agents operable to introduce the mutations comprise a sequence-specific endonuclease, donor templates and optionally guide RNAs, or comprise at least one nucleic acid construct that encodes a sequence-specific endonuclease, donor templates and optionally guide RNAs.

6. The method of claim 1, wherein the agents operable to introduce the at least one desired mutation in the target sequence comprise a guide RNA targeting the target sequence and the agents operable to introduce the mutation in the endogenous ALS gene comprise a guide RNA targeting the endogenous ALS1 gene or endogenous ALS2 gene or both, or wherein the agents operable to introduce the at least one desired mutation in the target sequence comprise at least one nucleic acid construct encoding a guide RNA targeting the target sequence and the agents operable to introduce the mutation in the endogenous ALS gene comprise at least one nucleic acid construct encoding a guide RNA targeting the endogenous ALS1 or endogenous ALS2 gene or both.

7. The method of claim 1, wherein the agents comprise nucleic acid constructs and said contacting comprises Agrobacterium-mediated transformation, particle bombardment, protoplast transformation, nanoparticle-mediated transfection or electroporation.

8. The method of claim 1, wherein step (c) comprises incubating an embryogenic cell suspension in the presence of an ALS inhibitor or step (c) comprises culturing embryos on embryo development medium (EDM) comprising an ALS inhibitor, optionally wherein:
step (c) comprises culturing embryos on embryo development media comprising an ALS inhibitor embryogenic cell suspension for 8-20 weeks, such as 8-16, 10-16, 10-14, 12-14 or around 13 weeks; or
step (c) comprises culturing embryos on embryo development media comprising an ALS inhibitor embryogenic cell suspension at a concentration of 10-80 μg/L, such as 10-70, 15-65, 15-55, 20-55, 20-50, 20-40, 20-30 or around 25 μg/L; or
step (c) comprises culturing embryos on embryo development media comprising an ALS inhibitor embryogenic cell suspension at a concentration of 10-80 μg/L, such as 10-70, 15-65, 15-55, 20-55, 20-50, 20-40, 20-30 or around 25 μg/L, for 8-20 weeks, such as 8-16, 10-16, 10-14, 12-14 or around 13 weeks.

9-10. (canceled)

11. The method of claim 8, comprising selecting at least one embryo following treatment with an ALS inhibitor and developing a banana plant in the absence of further treatment with an ALS inhibitor.

12. The method of claim 8, additionally comprising subsequently transferring embryos to an embryo development medium that does not comprise an ALS inhibitor.

13. The method of claim 1, comprising selecting at least one banana cell, such as an embryo, a banana tissue or banana plant that is resistant to treatment with an ALS inhibitor and physically separating them from cells, tissue or plants that are not resistant and optionally transferring the at least one selected cell, tissue or plant to a separate plate or growth medium, preferably without an ALS inhibitor.

14. The method of claim 1, comprising selecting at least one banana cell, such as an embryo, a banana tissue or banana plant that is resistant to treatment with an ALS inhibitor and subsequently selecting at least one banana cell, such as an embryo, a banana tissue or banana plant that also comprises the desired mutation in the target sequence, for example, by genotyping the cell, tissue or plant.

15. The method of claim 1, wherein the agents operable to introduce the at least one desired mutation in the target sequence and/or the agents operable to introduce the mutation in the endogenous ALS gene comprise a nucleic acid construct that additionally encodes a selectable marker, and wherein the method optionally comprises selecting an embryo or plant that does not carry the selectable marker.

16. The method of claim 1, wherein the endogenous acetolactate synthase gene is the endogenous acetolactate synthase 1 (ALS1) gene or the endogenous acetolactate synthase 2 (ALS2) gene.

17. The method of claim 1, wherein the mutation in the endogenous ALS gene is a substitution that introduces a substitution in the encoded amino acid sequence, preferably at Pro-187 in banana ALS1 or Pro-181 in ALS2, most preferably Pro187Ser in ALS1 or Pro181Ser in ALS2.

18. The method of claim 1, wherein the ALS inhibitor is a sulfonylurea, imidazolinone, triazolopyrimidine, pyrimidinyl oxybenzoate, or sulfonylamino carbonyl triazolinones, preferably wherein the ALS inhibitor is chlorsulfuron.

19. The method of claim 1, wherein step (c) comprises treating the banana cell with the ALS inhibitor and said treating provides a population of banana cells and at least 90%, such as at least 95%, 96%, 97%, 98% or 99%, of the cells in the population comprise mutated ALS, optionally wherein at least 1%, 5%, 10% or 15% of the cells do not comprise heterologous DNA, further optionally wherein at least 0.1% 1%, 2%, 3%, 4% or 5% of the cells comprise the mutation in the endogenous acetolactate synthase gene and comprise the desired mutation in the target sequence.

20. The method of claim 1, wherein the at least one banana embryo or plant generated by the method does not comprise heterologous DNA.

21. The method of claim 1, wherein the target sequence is ACO, ACS or PPO, such as ACO1, ACO2, ACS1, ACS2 or PPO2, or such as PPO1, PPO2, PPO8, PPO9 or PPO4.

22. The method of claim 1, further comprising growing a banana plant from at least one cell or tissue selected in step (c), optionally further comprising harvesting a fruit from said banana plant, optionally further comprising processing said fruit into a food product.

23. A banana cell, cell population, tissue or plant generated by the method according to claim 1.
